(19) United States
(12) Patent Application Publication
(10) Pub. No.: US 2026/0109674 A1
(43) Pub. Date: Apr. 23, 2026

(54) ALLOSTERIC MODULATION OF MUSCARINIC ACETYLCHOLINE RECEPTOR EFFICACY

(71) Applicants: The University of Toledo, Toledo, OH (US); The Penn State Research Foundation, University Park, PA (US)

(72) Inventors: William S. Messer, JR., Toledo, OH (US); Hazel Cunningham, Toledo, OH (US); Corey Widman, Toledo, OH (US); John Ellis, University Park, PA (US); Isaac Schiefer, Toledo, OH (US); Gina Kaup, Toledo, OH (US)

(73) Assignees: The University of Toledo, Toledo, OH (US); The Penn State Research Foundation, University Park, PA (US)

(21) Appl. No.: 19/117,060

(22) PCT Filed: Oct. 23, 2023
(86) PCT No.: PCT/US2023/035684

§ 371 Date: Mar. 30, 2025

Related U.S. Application Data
(60) Provisional application No. 63/424,542, filed on Nov. 11, 2022.

(51) Int. Cl. C07D 215/56; A61K 31/343; A61K 31/403; A61K 31/47; A61P 25/14; C07D 209/38; C07D 307/80

(52) U.S. Cl. CPC C07D 215/56; A61K 31/343; A61K 31/403; A61K 31/47; A61P 25/14; C07D 209/38; C07D 307/80

(57) ABSTRACT

Allosteric modulators of muscarinic receptors, and methods of making and using the same, are described.

### RELATED APPLICATIONS

This application claims priority to U.S. Provisional Application No. 63/424,542 filed under 35 U.S.C. § 111(b) on Nov. 11, 2022, the disclosure of which is incorporated herein by reference in its entirety for all purposes.

### STATEMENT REGARDING FEDERALLY SPONSORED RESEARCH

This invention was made with government support under Grant No. AG005214 awarded by the National Institutes of Health. The Government has certain rights in the invention.

### BACKGROUND

Several neurological disorders, including Alzheimer's disease, schizophrenia, autism spectrum disorders, and post-traumatic stress disorders, are associated with cognitive deficits. For example, autism spectrum disorders are characterized by persistent social and communication deficits, and restricted and repetitive behaviors. At the present time, there are no approved pharmaceutical treatments for the core symptoms of autism spectrum disorders, although antipsychotic and antidepressant drugs are often prescribed for treating other aspects of the disorders (for example, behavioral outbursts and anxiety). However, such compounds do not address core symptoms and can make things worse due to significant adverse effects.

The family of G protein-coupled receptors (GPCRs) is one of the most common drug targets in modern drug design. An estimated 35% of approved drugs in the United States and European Union interact with GPCRs. GPCRs are attractive targets due to their accessibility on the extracellular surface of cell membranes and involvement in a wide range of physiological responses in the central nervous system (CNS) and peripheral nervous system (PNS). However, GPCR signaling cascades are complex biochemical mechanisms that interact with a variety of intracellular enzymes which can complicate their study.

Muscarinic acetylcholine receptors (mAChRs) are acetycholine receptors which form G protein-coupled receptor complexes in the cell membranes of neurons. mAChRs are members of the class a GPCR family that respond to acetylcholine (ACh) and are classified into five distinct receptor subtypes (M₁, M₂, M₃, M₄, and M₅). The various subtypes have been found to be associated with regulating memory and cognitive function (M₁), drug reinforcement (M₅), locomotor activity (M₁/M₄), and cardiovascular/renal/gastro-intestinal function (M₂/M₃). The five receptor subtypes are characterized by the sequence similarity/identity of highly conserved regions of protein structure, especially amino acids within the seven transmembrane domains and in the membrane-proximal portions of the intracellular and extracellular loops. Each mAChR forms a complex with corresponding intracellular G proteins that when activated produces an intracellular signaling cascade. M_{1,3,5} receptors couple to Gα_{q/11} subunits, which stimulate phospholipase Cβ, leading to activation of protein kinase C and calcium mobilization; M_{2,4} receptors couple to Gα_{i/o} subunits, which inhibit adenylyl cyclase, thereby decreasing cAMP levels.

All of the mAChR subtypes feature two types of ligand binding, via orthosteric and allosteric sites. The orthosteric site is primarily responsible for receptor activation by the endogenous ligand (ACh) and other orthosteric agonists (e.g., oxotremorine and arecoline). Ligand binding to allosteric sites results in modulation of receptor activity. The initial generations of proposed drugs that targeted mAChRs included ACh precursors (e.g., choline), acetylcholinesterase inhibitors (e.g., tetrahydroaminoacridine), and mAChR agonists (e.g., pilocarpine), which directly or indirectly interact with the orthosteric binding site. Many CNS disorders involve altered activity of specific mAChR subtypes, and treatment of these disorders with subtype non-specific agents frequently results in adverse effects. In general, orthosteric ligands all exhibit poor selectivity between the different mAChR subtypes due to the orthosteric binding site being located in highly conserved regions of the receptor (within the transmembrane domains). Thus, there is a need in the art for new subtype-selective ligands.

### SUMMARY

Provided is a composition comprising Formula I:

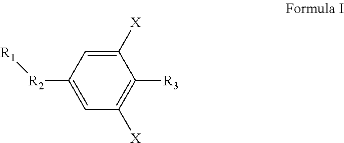

wherein each X is, independently, H, F, Cl, Br, I, OCH₃, CF₃, or OCF₃; R₁ is selected from

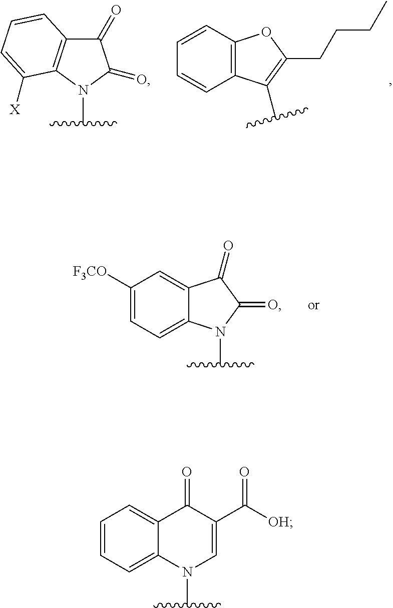

is selected from either

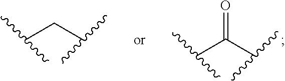

and R₃ is H, F, Br, OCF₃, OCH₃,

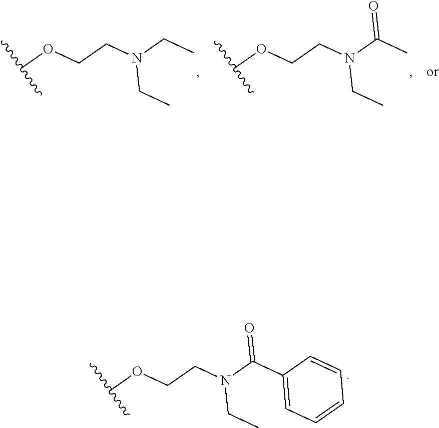

Also provided are salts, stereoisomers, racemates, hydrates, solvates, polymorphs, and prodrugs of Formula I.

In certain embodiments, the composition comprises compound 5a:

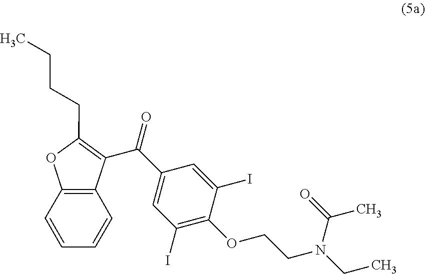 [SMILES: CCCCc1oc2ccccc2c1C(=O)c1cc(I)c(OCCN(CC)C(C)=O)c(I)c1]

In certain embodiments the composition comprises compound 5b:

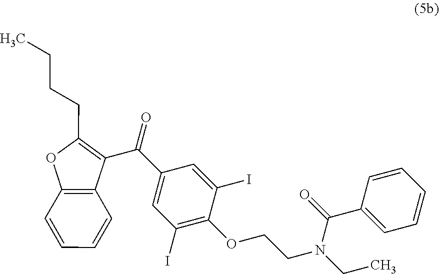 [SMILES: CCCCc1oc2ccccc2c1C(=O)c1cc(I)c(OCCN(CC)C(=O)c2ccccc2)c(I)c1]

In certain embodiments the composition comprises compound 7:

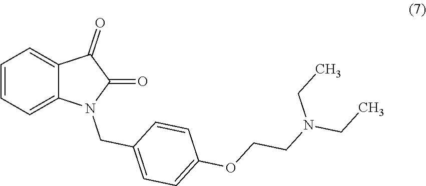 [SMILES: CCN(CC)CCOc1ccc(CN2C(=O)C(=O)c3ccccc32)cc1]

In certain embodiments, the composition comprises compound 8a:

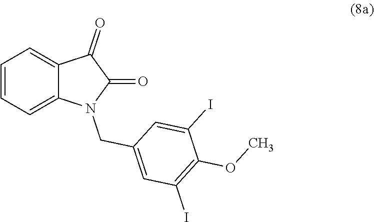 [SMILES: COc1c(I)cc(CN2C(=O)C(=O)c3ccccc32)cc1I]

In certain embodiments, the composition comprises compound 8c:

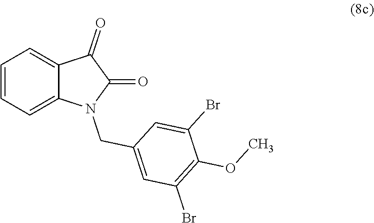 [SMILES: COc1c(Br)cc(CN2C(=O)C(=O)c3ccccc32)cc1Br]

In certain embodiments, the composition comprises compound 8l:

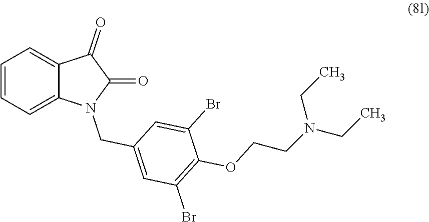 [SMILES: CCN(CC)CCOc1c(Br)cc(CN2C(=O)C(=O)c3ccccc32)cc1Br]

In certain embodiments, the composition comprises compound 8j:

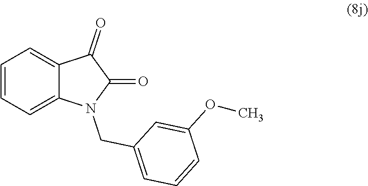 [SMILES: COc1cccc(CN2C(=O)C(=O)c3ccccc32)c1]

In certain embodiments, the composition comprises compound 8e:

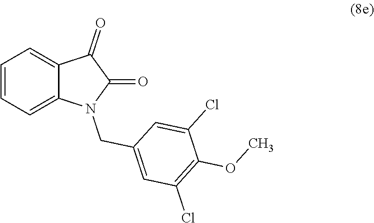 [SMILES: COc1c(Cl)cc(CN2C(=O)C(=O)c3ccccc32)cc1Cl]

In certain embodiments, the composition comprises compound 8g:

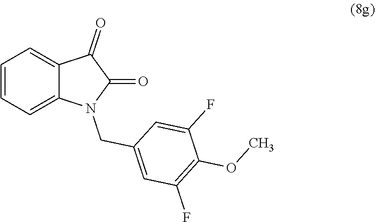 [SMILES: COc1c(F)cc(CN2C(=O)C(=O)c3ccccc32)cc1F]

In certain embodiments, the composition comprises compound GK-1-44:

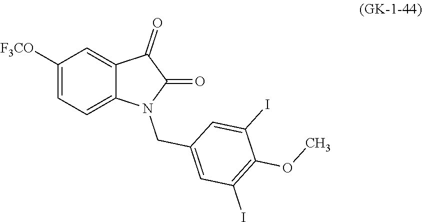 [SMILES: COc1c(I)cc(CN2C(=O)C(=O)c3cc(OC(F)(F)F)ccc32)cc1I]

In certain embodiments, the composition comprises compound GK-1-48:

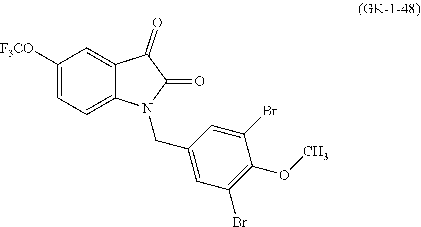 [SMILES: COc1c(Br)cc(CN2C(=O)C(=O)c3cc(OC(F)(F)F)ccc32)cc1Br]

In certain embodiments, the composition comprises compound GK-1-47:

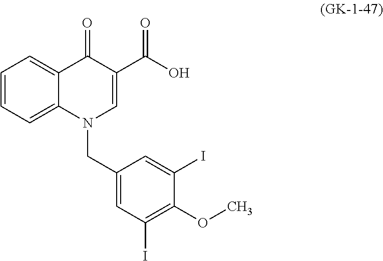 [SMILES: COc1c(I)cc(Cn2cc(C(=O)O)c(=O)c3ccccc32)cc1I]

In certain embodiments, the composition comprises compound GK-1-50:

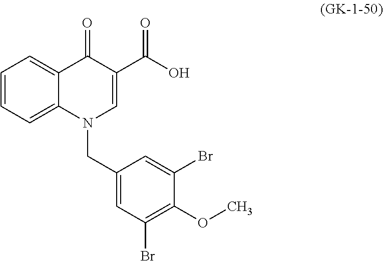 [SMILES: COc1c(Br)cc(Cn2cc(C(=O)O)c(=O)c3ccccc32)cc1Br]

Further provided is a composition comprising Formula II:

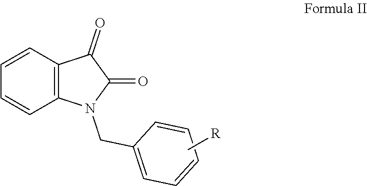

wherein R is alkoxy or substituted alkoxy, and optionally, the R-substituted phenyl ring is further substituted with one or more halogens. Also provided are salts, stereoisomers, racemates, hydrates, polymorphs, and prodrugs of Formula II.

In certain embodiments, R is methoxy. In particular embodiments, the methoxy is substituted. In certain embodiments, R is ethoxy.

Further provided is a composition comprising Formula III:

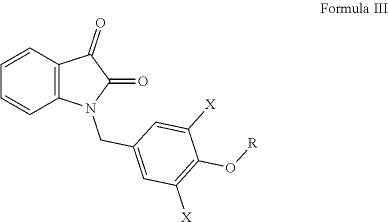

wherein each X is, independently, a halogen; and R is alkyl. Also provided are salts, stereoisomers, racemates, hydrates, polymorphs, and prodrugs of Formula III.

Further provided is a composition comprising Formula IV:

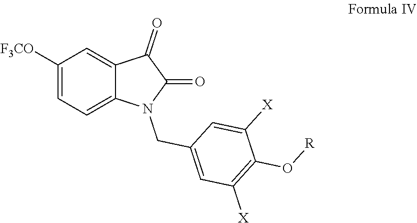

wherein each X is, independently, a halogen; and R is alkyl. Also provided are salts, stereoisomers, racemates, hydrates, polymorphs, and prodrugs of Formula IV.

Further provided is a composition comprising Formula V:

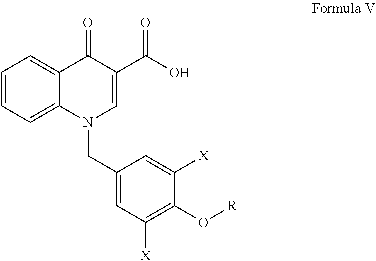

wherein each X is, independently, a halogen; and R is alkyl. Also provided are salts, stereoisomers, racemates, hydrates, polymorphs, and prodrugs of Formula V.

In certain embodiments, R is methyl. In certain embodiments, each X is the same halogen. In certain embodiments, each X is the same halogen and R is methyl.

Further provided is a method of modulating ACh activity at M₁ muscarinic receptors, the method comprising contacting cells expressing M₁ muscarinic receptors with an effective amount of a compound of Formula I to modulate ACh activity at the M₁ muscarinic receptors expressed by the cells:

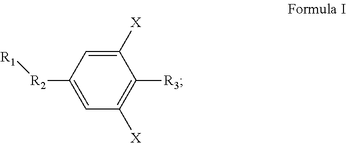

wherein each X is, independently, H, F, Cl, Br, I, OCH₃, CF₃, or OCF₃; R₁ is selected from

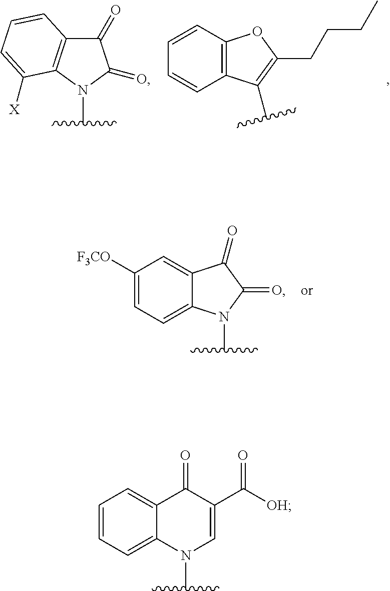

R₂ is selected from either

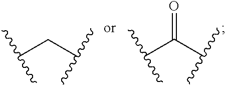

and R₃ is H, F, Br, OCF₃, OCH₃.

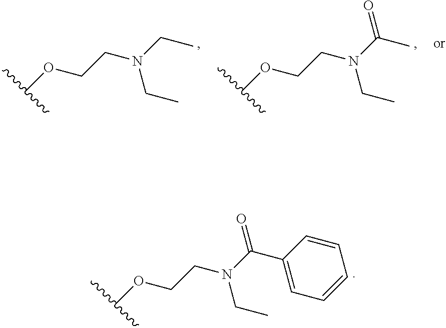

In certain embodiments, ACh efficacy is enhanced. In certain embodiments, ACh potency is enhanced.

In certain embodiments, the compound is compound 5a:

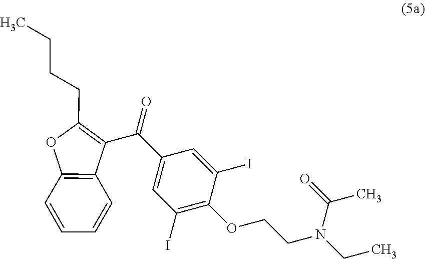 [SMILES: CCCCc1oc2ccccc2c1C(=O)c1cc(I)c(OCCN(CC)C(C)=O)c(I)c1]

In certain embodiments the compound is compound 5b:

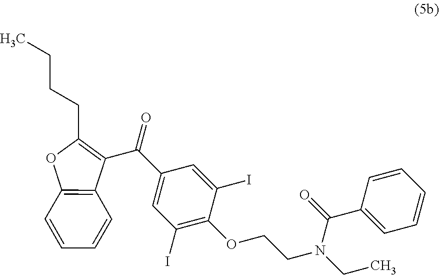 [SMILES: CCCCc1oc2ccccc2c1C(=O)c1cc(I)c(OCCN(CC)C(=O)c2ccccc2)c(I)c1]

In certain embodiments the compound is compound 7:

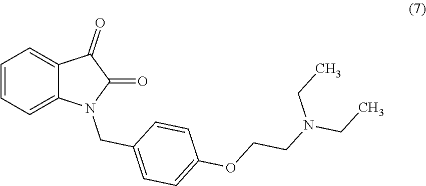 [SMILES: CCN(CC)CCOc1ccc(CN2C(=O)C(=O)c3ccccc32)cc1]

In certain embodiments, the compound is compound 8a:

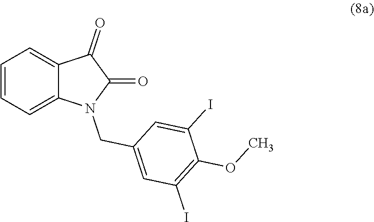 [SMILES: COc1c(I)cc(CN2C(=O)C(=O)c3ccccc32)cc1I]

In certain embodiments, the compound is compound 8c:

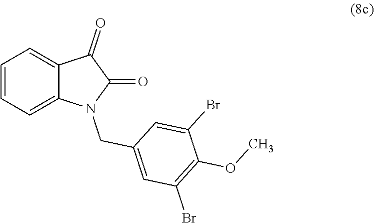 [SMILES: COc1c(Br)cc(CN2C(=O)C(=O)c3ccccc32)cc1Br]

In certain embodiments, the compound is compound 8e:

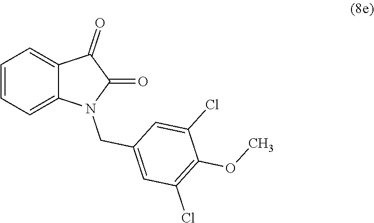 [SMILES: COc1c(Cl)cc(CN2C(=O)C(=O)c3ccccc32)cc1Cl]

In certain embodiments, the compound is compound 8g:

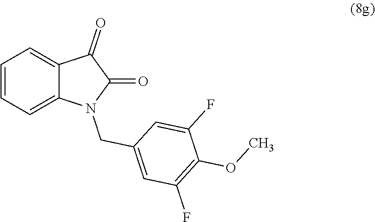 [SMILES: COc1c(F)cc(CN2C(=O)C(=O)c3ccccc32)cc1F]

In certain embodiments, the compound is compound 8l:

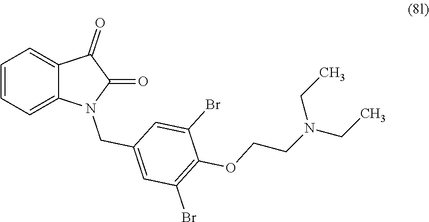 [SMILES: CCN(CC)CCOc1c(Br)cc(CN2C(=O)C(=O)c3ccccc32)cc1Br]

In certain embodiments, the compound is compound 8j:

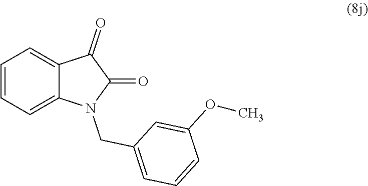 [SMILES: COc1cccc(CN2C(=O)C(=O)c3ccccc32)c1]

In certain embodiments, the compound is GK-1-44:

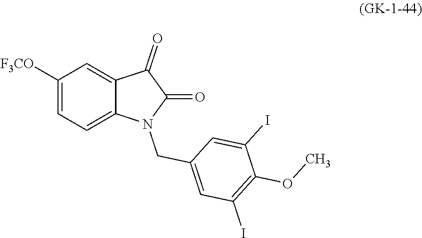 [SMILES: COc1c(I)cc(CN2C(=O)C(=O)c3cc(OC(F)(F)F)ccc32)cc1I]

In certain embodiments, the compound is GK-1-48:

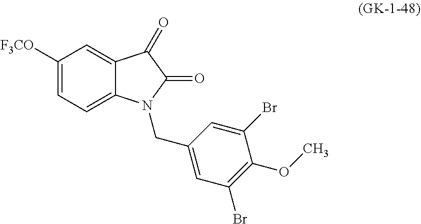 [SMILES: COc1c(Br)cc(CN2C(=O)C(=O)c3cc(OC(F)(F)F)ccc32)cc1Br]

In certain embodiments, the compound is GK-1-47:

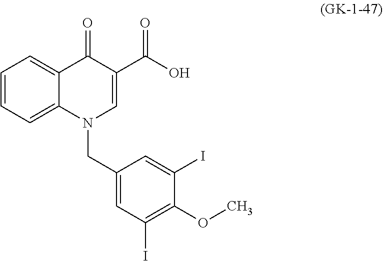 [SMILES: COc1c(I)cc(Cn2cc(C(=O)O)c(=O)c3ccccc32)cc1I]

In certain embodiments, the compound is GK-1-50:

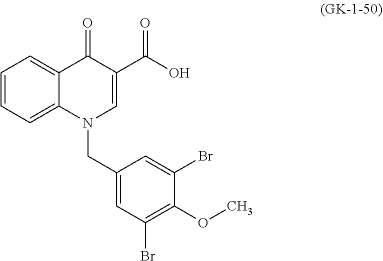 [SMILES: COc1c(Br)cc(Cn2cc(C(=O)O)c(=O)c3ccccc32)cc1Br]

Further provided is a method of decreasing repetitive behaviors in a subject, the method comprising administering an effective amount of a compound of Formula I to a subject and decreasing repetitive behaviors in the subject:

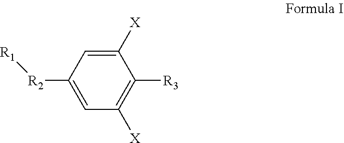

wherein each X is, independently, H, F, Cl, Br, I, OCH₃, CF₃, or OCF₃; R₁ is selected from

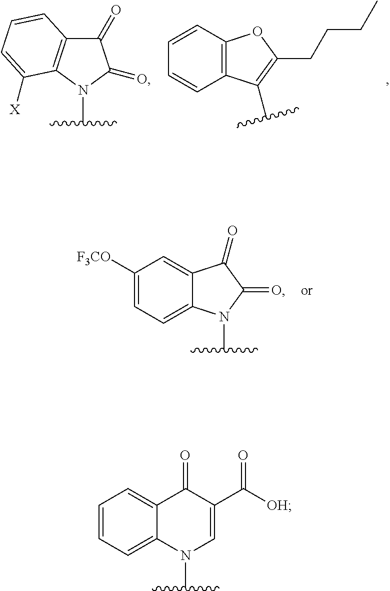

R₂ is selected from either

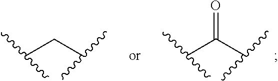

and R₃ is H, F, Br, OCF₃, OCH₃,

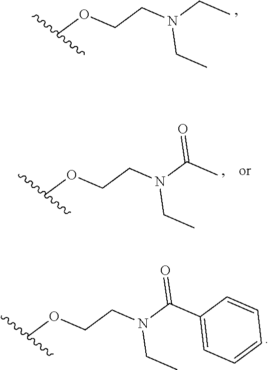

In certain embodiments, the compound is compound 8a:

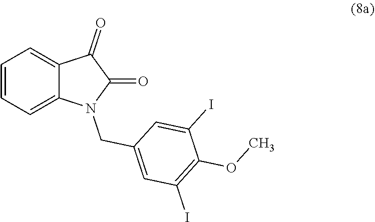 [SMILES: COc1c(I)cc(CN2C(=O)C(=O)c3ccccc32)cc1I]

In certain embodiments, the compound is compound 8c:

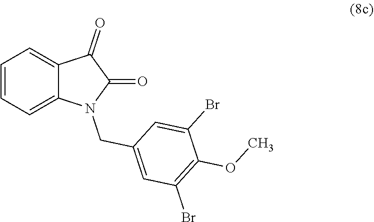 [SMILES: COc1c(Br)cc(CN2C(=O)C(=O)c3ccccc32)cc1Br]

In certain embodiments, the compound is compound 8e:

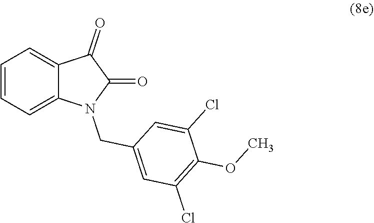 [SMILES: COc1c(Cl)cc(CN2C(=O)C(=O)c3ccccc32)cc1Cl]

In certain embodiments, the compound is compound 8g:

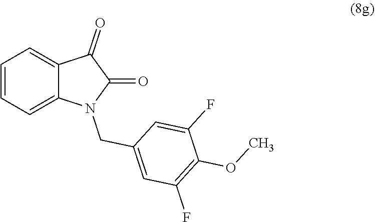 [SMILES: COc1c(F)cc(CN2C(=O)C(=O)c3ccccc32)cc1F]

In certain embodiments, the compound is compound 8l:

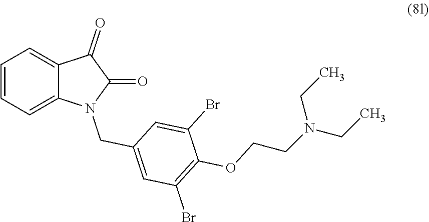 [SMILES: CCN(CC)CCOc1c(Br)cc(CN2C(=O)C(=O)c3ccccc32)cc1Br]

In certain embodiments, the compound is compound 8j:

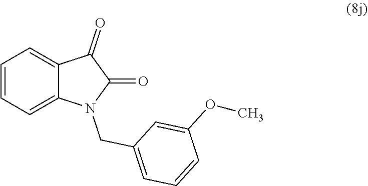 [SMILES: COc1cccc(CN2C(=O)C(=O)c3ccccc32)c1]

In certain embodiments, the compound is GK-1-48:

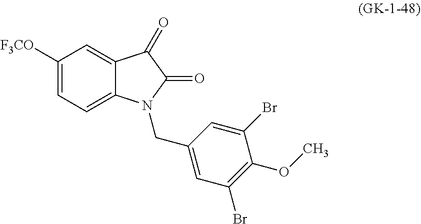 [SMILES: COc1c(Br)cc(CN2C(=O)C(=O)c3cc(OC(F)(F)F)ccc32)cc1Br]

Further provided is a method of treating a neurological disorder, the method comprising administering to a subject having a neurological disorder an effective amount of a compound of Formula I to treat the neurological disorder:

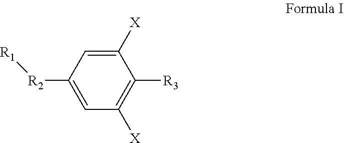

wherein each X is, independently, H, F, Cl, Br, I, OCH₃, CF₃, or OCF₃; R₁ is selected from

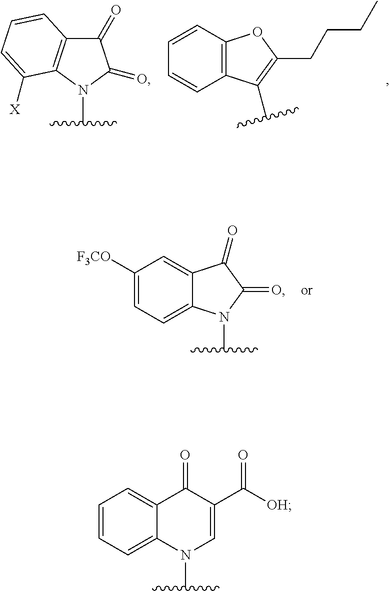

R₂ is selected from either

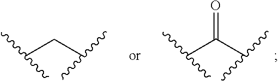

and R₃ is H, F, Br, OCF₃, OCH₃,

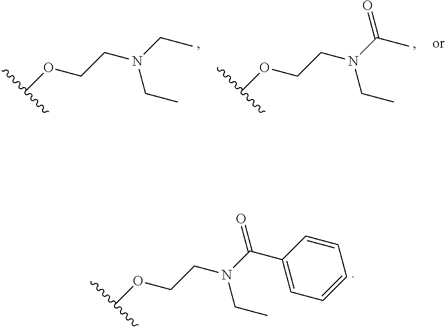

In certain embodiments, the neurological disorder is Alzheimer's disease, Parkinson's disease, schizophrenia, autism spectrum disorders, substance abuse, or post-traumatic stress disorder.

In certain embodiments, the compound is a compound of Formula II:

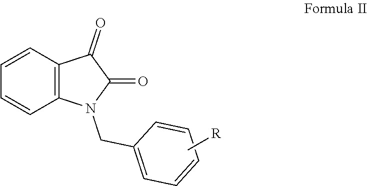

where R is an alkoxy or substituted alkoxy group, and optionally, the R-substituted phenyl ring is further substituted with one or more halogens.

In certain embodiments, the compound is a compound of Formula III:

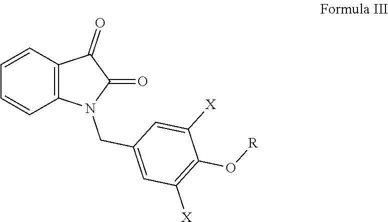

where each X is, independently, a halogen; and R is alkyl.

In certain embodiments, the compound is a compound of Formula IV:

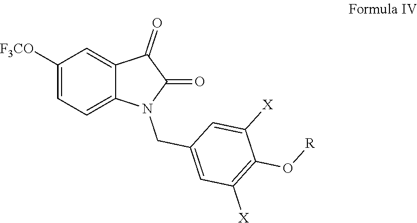

wherein each X is, independently, a halogen; and R is alkyl.

In certain embodiments, the compound is a compound of Formula V:

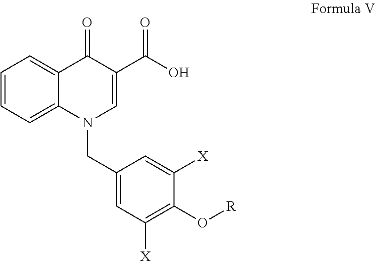

where each X is, independently, a halogen; and R is alkyl.

In certain embodiments, the compound is compound 7:

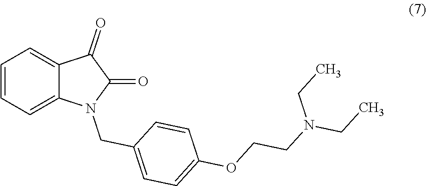 [SMILES: CCN(CC)CCOc1ccc(CN2C(=O)C(=O)c3ccccc32)cc1]

In certain embodiments, the neurological disorder is post-traumatic stress disorder, and the compound is compound 7.

In certain embodiments, the compound is compound 8a:

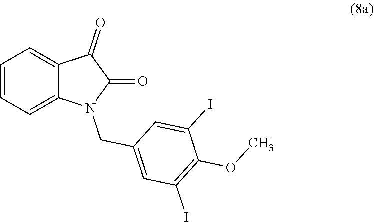 [SMILES: COc1c(I)cc(CN2C(=O)C(=O)c3ccccc32)cc1I]

In certain embodiments, the neurological disorder is Alzheimer's disease, and the compound is compound 8a.

In certain embodiments, the compound is compound 8c:

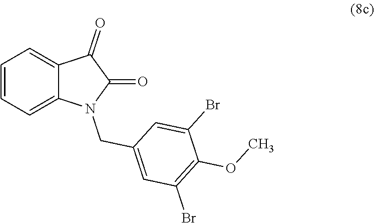 [SMILES: COc1c(Br)cc(CN2C(=O)C(=O)c3ccccc32)cc1Br]

In certain embodiments, the compound is compound 8e:

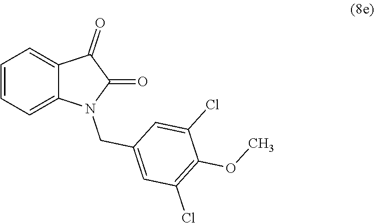 [SMILES: COc1c(Cl)cc(CN2C(=O)C(=O)c3ccccc32)cc1Cl]

In certain embodiments, the compound is compound 8g:

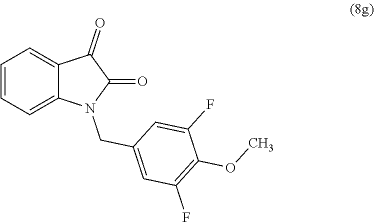 [SMILES: COc1c(F)cc(CN2C(=O)C(=O)c3ccccc32)cc1F]

In certain embodiments, the compound is compound 8l:

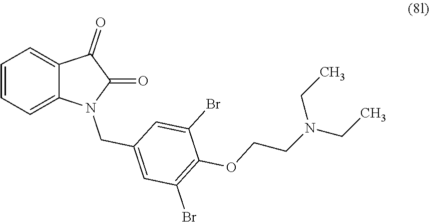 [SMILES: CCN(CC)CCOc1c(Br)cc(CN2C(=O)C(=O)c3ccccc32)cc1Br]

In certain embodiments, the compound is compound 8j:

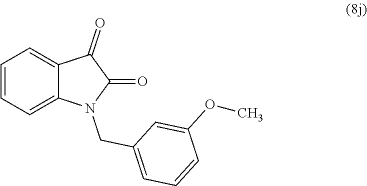 [SMILES: COc1cccc(CN2C(=O)C(=O)c3ccccc32)c1]

In certain embodiments, the compound is GK-1-44:

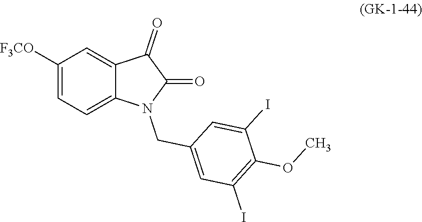 [SMILES: COc1c(I)cc(CN2C(=O)C(=O)c3cc(OC(F)(F)F)ccc32)cc1I]

In certain embodiments, the compound is GK-1-48:

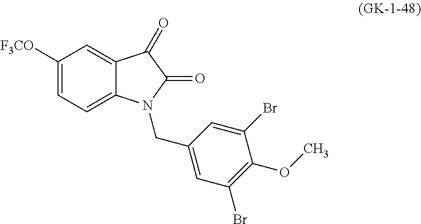 [SMILES: COc1c(Br)cc(CN2C(=O)C(=O)c3cc(OC(F)(F)F)ccc32)cc1Br]

In certain embodiments, the compound is GK-1-47:

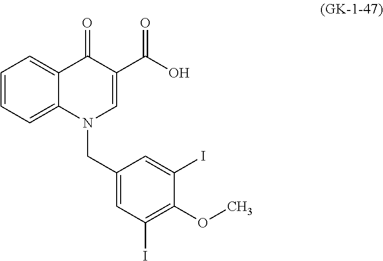 [SMILES: COc1c(I)cc(Cn2cc(C(=O)O)c(=O)c3ccccc32)cc1I]

In certain embodiments, the compound is GK-1-50:

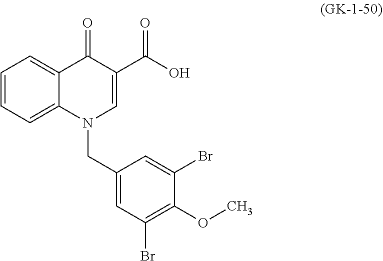 [SMILES: COc1c(Br)cc(Cn2cc(C(=O)O)c(=O)c3ccccc32)cc1Br]

Further provided is a pharmaceutical composition comprising a pharmaceutically acceptable carrier, diluent, or adjuvant; and a compound of Formula I:

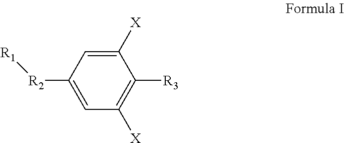

wherein each X is, independently, H, F, Cl, Br, I, OCH₃, CF₃, or OCF₃; R₁ is selected from

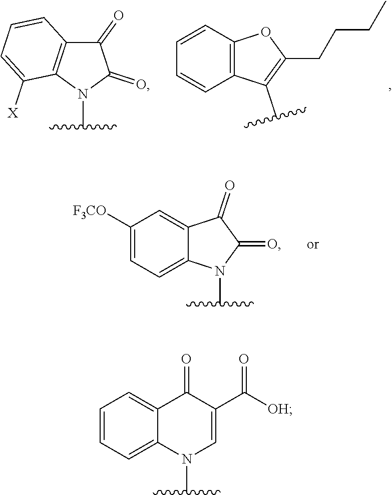

R₂ is selected from either

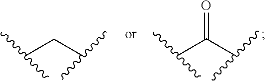

and R₃ is H, F, Br, OCF₃, OCH₃,

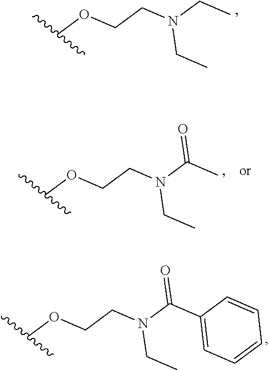

or for a salt, stereoisomer, racemate, hydrate, solvate, polymorph, or prodrug of Formula I.

Further provided is a method of making a M₁ muscarinic receptor modulator compound, the method comprising alkylating a benzaldehyde with an unsubstituted phenol in the presence of a base to produce an alkylated benzaldehyde; reducing the alkylated benzaldehyde with a reducing agent to produce a benzyl alcohol; halogenating the benzyl alcohol with a halogenation agent to produce a benzyl halide; and N-alkylating isatin with the benzyl halide to obtain a M₁ muscarinic receptor modulator compound. In certain embodiments, the halogenation agent comprises thionyl chloride.

Further provided is a method of making a M₁ muscarinic receptor modulator compound, the method comprising brominating para-tolyl acetate with a brominating agent to obtain a benzyl bromide; alkylating the benzyl bromide with isatin to obtain an acetylated benzyl dione; deacetylating the acetylated benzyl dione to obtain a hydroxy benzyl dione; and subjecting the hydroxy benzyl dione to a Williamson ether synthesis with an alkyl halide to obtain a M₁ muscarinic receptor modulator compound. In certain embodiments, the brominating agent is N-bromosuccinimide.

Further provided is a method of making a M₁ muscarinic receptor modulator compound, the method comprising n-dealkylating amiodarone with a chloroethyl chloroformate to obtain a carbamate intermediate; allowing the carbamate intermediate to decompose into a decomposed intermediate; and reacting the decomposed intermediate with an anhydride to obtain a M₁ muscarinic receptor modulator compound.

Further provided is a method of making a M₁ muscarinic receptor modulating compound, the method comprising alkylating a benzaldehyde with an unsubstituted phenol in the presence of a base to produce an alkylated benzaldehyde; reducing the alkylated benzaldehyde with a reducing agent to produce a benzyl alcohol; halogenating the benzyl alcohol with a halogenation agent to produce a benzyl halide; and N-alkylating 5-trifluoromethoxy-isatin with the benzyl halide to obtain the M₁ muscarinic receptor modulator compound.

Further provided is a method of making a M₁ muscarinic receptor modulating compound, the method comprising alkylating a benzaldehyde with an unsubstituted phenol in the presence of a base to produce an alkylated benzaldehyde; reducing the alkylated benzaldehyde with a reducing agent to produce a benzyl alcohol; halogenating the benzyl alcohol with a halogenation agent to produce a benzyl halide; N-alkylating isatin with the benzyl halide to obtain an N-alkylated isatin; and subjecting the N-alkylated isatin to an ester hydrolysis to obtain the M₁ muscarinic receptor modulator compound.

Further provided is a use of a compound of Formula I to increase the activity of acetylcholine without directly activating M₁ muscarinic receptors:

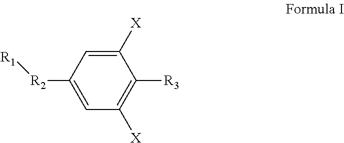

wherein each X is, independently, H, F, Cl, Br, I, OCH₃, CF₃, or OCF₃; R₁ is selected from

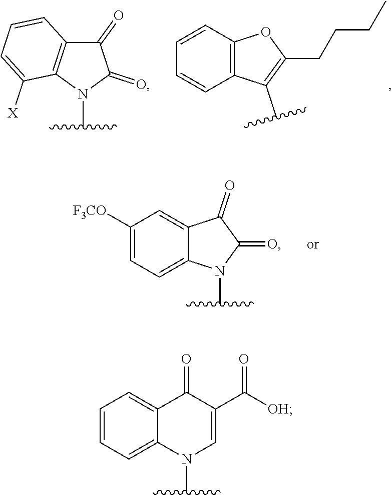

R₂ is selected from either

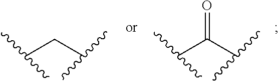

an R₃ is H, F, Br, OCF₃, OCH₃,

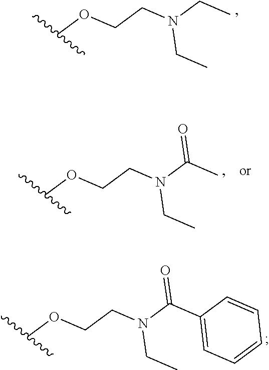

or a salt, stereoisomer, racemate, hydrate, solvate, polymorph, or prodrug thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Chart showing some non-limiting examples of M₁ receptor modulator compounds in accordance with the present disclosure.

FIG. 2A: Examples of known positive allosteric modulators at M₁ muscarinic receptors.

FIG. 2B: Examples of known positive allosteric modulators that enhance ACh efficacy (exhibit positive β) at M₁ muscarinic receptors.

FIGS. 3A-3B: Stimulation of arachidonic acid release in CHO cells expressing M₁ muscarinic receptors by ACh alone and in the presence of either 10 μM 5a (FIG. 3A) or 10 μM 5b (FIG. 3B).

FIG. 4: Scheme 1, depicting the synthesis of compound 7. (i) NBS [1.3 eq], AIBN [cat.], CCl₄, 76° C., 2 hrs.; (ii) isatin [0.77 eq]], K₂CO₃ [1.54 eq], KI [0.77 eq], DMF, RT, 4 hrs.; (iii) K₂CO₃ [2.0 eq], MeOH, RT, 2 hrs.; (iv) ClEtN(Et₂)·HCl [1.5 eq], Cs₂CO₃ [2.0 eq], NaI [cat.], DMF, 60° C., 2 hrs.

FIG. 5: Stimulation of arachidonic acid release in CHO cells expressing M₁ muscarinic receptors by ACh alone and in the presence of compound 7. Compound 7 significantly enhances ACh efficacy, yet inhibits potency, at M₁ receptors expressed in CHO cells.

FIG. 6: Scheme 2, depicting the synthesis of compounds 8a-8l. (i) alkyl-halide [1.5 eq], Cs₂CO₃ [2.0 eq], NaI [cat.], DMF, 60° C., 2 hrs.; (ii) NaBH₄ [1.5 eq], MeOH, RT, 2 hrs.; (iii) SOCl₂ [1.7 eq], pyridine [1.3 eq], DCM, 0° C., 16 hrs.; (iv) isatin [0.77 eq], K₂CO₃ [1.54 eq], KI [0.77 eq], DMF, RT, 16 hrs.

FIG. 7: Modulation of ACh activity at M₁ muscarinic receptors expressed in CHO cells by compound 8a. Compound 8a dramatically enhances ACh efficacy at M₁ receptors expressed in CHO cells.

FIG. 8: Modulation of ACh activity at M₁ muscarinic receptors expressed in CHO cells by 7, 8a, 8j, and 8c. Summary of effects of allosteric modulators on the stimulation of [³H]-arachidonic acid release by acetylcholine at M₁ muscarinic receptors expressed in CHO cells.

FIG. 9: Non-limiting example isatin derivatives of amiodarone.

FIGS. 10A-10G: Locomotor behavior following administration of BQCA (FIG. 10A) and 8a (FIG. 10B). FIGS. 10C-10E show the distance moved during the 30-minute spontaneous swimming period following administration of BQCA (FIG. 10C) and 8a (FIG. 10E). FIGS. 10D-10F show repetitive behaviors (as measured by angular velocity) during the 30-minute spontaneous swimming period following administration of BQCA (FIG. 10D) and 8a (FIG. 10F). Increases in angular velocity reflect a decrease in repetitive behaviors. FIG. 10G shows repetitive behaviors as measured by turn angle during spontaneous swimming period following administration of 8a. In other words, FIGS. 10E-10G show the effects of 8a on spontaneous swimming behavior (FIG. 10E), angular velocity (FIG. 10F), and turn angle (FIG. 10G) in zebrafish. *p<0.05; **p<0.01; ***p<0.001.

FIGS. 11A-11C: Effects of 8j on spontaneous swimming behavior (FIG. 11A), angular velocity (FIG. 11B), and turn angle (FIG. 11C) in zebrafish. *p<0.05; **p<0.01; ***p<0.001.

FIGS. 12A-12C: Effects of 8c on A) spontaneous swimming behavior (FIG. 12A), angular velocity (FIG. 12B), and turn angle (FIG. 13C) in zebrafish. *p<0.05; **p<0.01; ***p<0.001.

FIGS. 13A-13C: Effects of 7 on spontaneous swimming behavior (FIG. 14A), angular velocity (FIG. 13B), and turn angle (FIG. 13C) in zebrafish. *p<0.05; **p<0.01; ***p<0.001.

FIGS. 14A-14C: Results of screen for activity at M₁ receptors using compounds 7, 8a, and 8c alone and in the presence of low and high [ACh].

FIG. 15: Non-limiting example isatin derivatives of amiodarone.

FIG. 16: Non-selective potency PAM (α>1) (top left); M₁ selective potency PAM (α>1) (top right); overlapping structural motif (middle); non-selective efficacy PAMs (β>1) (bottom left); non-selective efficacy NAM (β<1) (bottom right).

FIG. 17: Structural comparison of amiodarone, 5a, and 5b.

FIG. 18: Modulation of ACh response by BQCA and amiodarone. Release of [³H]AA by the indicated concentrations of ACh from CHO cells expressing M₁ muscarinic receptors was measured in the presence and absence of 10 μM BQCA (n=3) and 30 μM amiodarone (n=4). In order to minimize the agonist effect of BQCA, these assays included pretreatment of the cells with 0.3 μM phenoxybenzamine.

FIGS. 19A-19C: Modulation of ACh response by allosteric modulators. Assays were conducted as in FIG. 16, except that cells were not pretreated with phenoxybenzamine. FIG. 19A shows BQCA (n=2), amiodarone (n=2), 2 (n=4), and 1 (n=2) were each included at a concentration of 10 μM.

FIG. 19B shows amiodarone (n=2), dronedarone (n=4), 3 (n=2), and 4 (n=2) were each included at a concentration of 10 μM. FIG. 19C shows structures of compounds included in the figure.

FIG. 20: Scheme 3, depicting the synthesis of analogs 5a, 5b. (i) ACE-Cl [5.0 eq], 1,2-DCE, 0° C., 1 hr.; (ii) 1,2-DCE, 80° C., 2 hrs.; (iii) MeOH, 80° C., 1 hr.; (iv) anhydride [2.0 eq], Et₃N [2.0 eq], DMAP [cat.], DCM, 0° C. to RT, 16 hrs.

FIG. 21: Scheme 4, depicting the synthesis of compounds 6a-6c. (i) alkyl-halide [1.5 eq], Cs₂CO₃ [2.0 eq]. NaI [cat.], DMF. 60° C., 2 hrs.

FIG. 22: Design of allosteric modulator hybrids using the isatin heteroaromatic core from VU0119498 and structural features of interest of amiodarone.

FIG. 23: Modulation of ACh response by allosteric modulators. Assays were conducted as in FIG. 18. Each modulator was included at 10 μM. Data for 7 and 8a represent 10-12 experiments; data for 8c and 8l represent 2 experiments.

FIG. 24: Stimulation of |³H|-arachidonic acid release by ACh at M₁ muscarinic receptors expressed in CHO cells in the absence and presence of 8a from 0.01-10 μM. Data are presented as the fraction of the maximal response produced by 1.0 mM ACh. Curves are based on the allosteric operational model; see Table 2 for best-fit parameters.

### DETAILED DESCRIPTION

Throughout this disclosure, various publications, patents, and published patent specifications may be referenced by an identifying citation. The disclosures of these publications, patents, and published patent specifications are hereby incorporated by reference into the present disclosure in their entirety to more fully describe the state of the art to which this invention pertains.

Modification of M₁ muscarinic receptor activity is useful for treating diseases associated with altered cholinergic signaling such as Alzheimer's disease (AD), Parkinson's disease (PD), schizophrenia, autism spectrum disorders (ASD), dementia, and various other CNS disorders. However, the complexity of the central nervous system and the various types of pathological states associated with neurological disorders may require the ability to alter neuronal signaling to a greater extent than conventionally possible. While M₁ muscarinic receptor positive allosteric modulators (PAMs) that increase ACh potency are of possible use in enhancing the effects of ACh at low concentrations, there may be instances where it is desirable to enhance the activity of ACh at higher concentrations. Effective treatments may involve shaping neuronal signaling to fine-tune ACh activity beyond just enhancing potency.

The allosteric binding site(s) of the different mAChR subtypes are located in comparatively less highly conserved regions of the receptors (within the extracellular loops); thus, targeting allosteric sites is a useful strategy for the development of subtype-selective ligands. Models of allosteric regulation of G protein-coupled receptors (including muscarinic receptors) indicate it is possible to modify receptor activity in multiple ways, including 1) enhancing or decreasing agonist potency, 2) elevating or inhibiting agonist efficacy, and 3) directly activating or inhibiting receptor activity by allosteric ligands. Important parameters for assessing each of these distinct allosteric modulating effects include 1) the cooperativity factor α, reflecting changes in agonist affinity, 2) the cooperativity factor β, reflecting changes in agonist efficacy, 3) intrinsic efficacy τB, and 4) the equilibrium dissociation constant KB that reflects the affinity of the ligand for the allosteric binding site. Using these characteristics, allosteric ligands are subsequently categorized as either positive allosteric modulators (PAMs) which enhance potency or efficacy; negative allosteric modulators (NAMs) which diminish potency or efficacy; silent allosteric modulators (SAMs) which bind to the allosteric site but have no effect; and intrinsic allosteric ligands (IALs) which demonstrate allosteric agonist properties. The activity of allosteric ligands that exclusively modulate orthosteric effects are dependent on simultaneous binding of the orthosteric ligand to the receptor. This tertiary complex results in allosteric modulators preserving the endogenous spatiotemporal specificity of the system. Allosteric modulators also exhibit a maximal “ceiling effect” regulated by the concentration of the orthosteric ligand.

Activation of cholinergic pathways, elevating acetylcholine (ACh) activity, may be useful in treating neurological disorders. Current approaches include acetylcholinesterase inhibitors, muscarinic agonists, and allosteric modulators of muscarinic receptor subtypes. However, acetylcholinesterase inhibitors and muscarinic agonists have limited selectivity and generally enhance ongoing activity. Existing positive allosteric modulators primarily enhance ACh potency.

Allosteric modulators that modify receptor activity appropriately are useful as therapeutics for CNS disorders with an array of mAChR activity profiles. Positive allosteric modulation of efficacy has possible therapeutic implications for disorders involving low mAChR expression or low mAChR activity. Such is the case with approximately 25% of schizophrenia patients that have 75% fewer M₁ receptors than healthy patients. Enhancement of M₁ muscarinic receptor activity could alleviate cognitive deficits associated with schizophrenia and related disorders. On the other hand, psychopharmacological studies show that administration of the muscarinic antagonist scopolamine provides an effective treatment for major depressive disorder. Similar therapeutic outcomes for related cholinergic hypersensitivities may be achieved with greater selectivity by negative allosteric modulation of mAChRs.

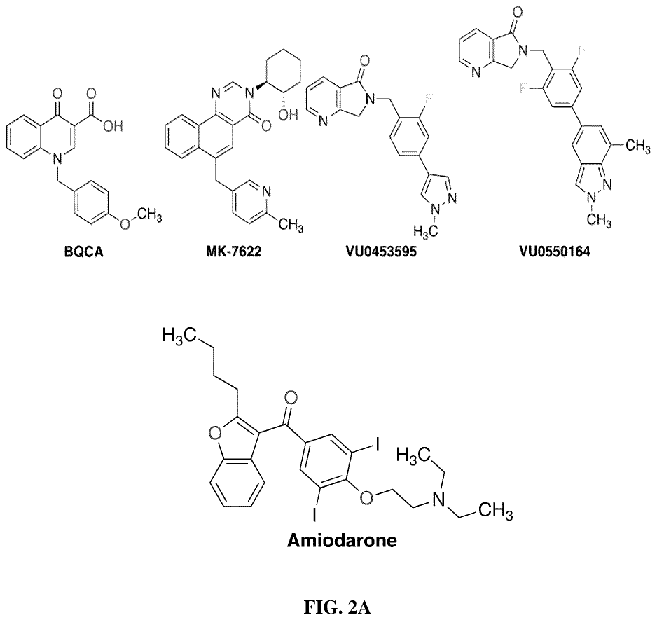

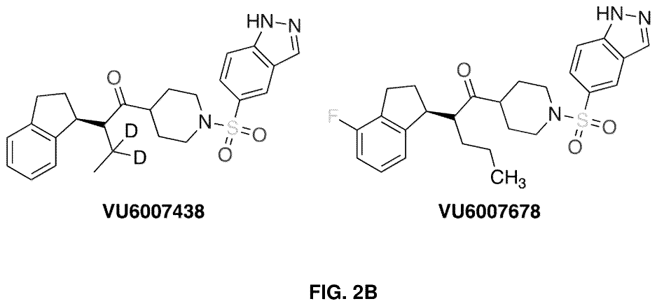

Muscarinic receptors mediate myriad responses of acetylcholine (ACh) in the central nervous system and in peripheral tissues. M₁ muscarinic receptors represent an important target due to their localization in the cerebral cortex, hippocampus, and neostriatum—brain regions implicated in learning, memory, and cognitive function. Compounds that modulate the activity of muscarinic receptors may be useful in the treatment of neurological disorders such as Alzheimer's disease, schizophrenia, and autism spectrum disorders. There are some previously identified compounds that act as positive allosteric modulators of M₁ muscarinic receptors by enhancing the potency of ACh. In particular, compounds such as BQCA, MK-7622, VU0453595, and VU 0550164 (depicted in FIG. 2A) enhance the potency of ACh at M₁ muscarinic receptors, functioning as positive allosteric modulators (PAMs). All of the compounds shown in FIG. 2A exhibit the ability to enhance agonist potency (positive α), as they all shift ACh dose response curves to the left, enhancing ACh activity at low concenrations. Two of these compounds, BQCA and MK-7622, also display intrinsic agonist activity (τB), while VU0453595 and VU 0550164 lack intrinsic agonist activity. Allosteric ligands with intrinsic agonist activity may be associated with an increased incidence of adverse effects and reduced efficacy in vivo.

A few allosteric modulators have been identified that can enhance ACh efficacy, including VU6007438 and VU6007678 (shown in FIG. 2B), which potentiate ACh responses at M₁, M₃, and M₅ muscarinic receptors. Their effects are not limited to elevating ACh efficacy, however, as these compounds also exhibit positive cooperativity in enhancing ACh potency and act as allosteric agonists in the absence of ACh. Amiodarone, a drug that is used in treating cardiac arrhythmias, is unusual in that it is one of the few allosteric ligands that enhance ACh efficacy (positive β) with minimal effects on ACh potency (positive α) and relatively low intrinsic agonist activity (τB). Amiodarone enhances responses at high ACh concentrations (enhance efficacy). In contrast, most positive allosteric modulators (e.g., BQCA) enhance activity at lower concentrations (enhance potency). The effect of enhanced ACh efficacy by amiodarone is distinct from that produced by BQCA, which enhances ACh potency. The effects are additive, indicating that they act at different sites on M₁ muscarinic receptors. Unfortunately, amiodarone exhibits a number of pharmacological effects, including modulation of ion channels, and is not suitable for development as a therapeutic agent for neurological disorders due to its large molecular size (molecular weight of 645.32) and high lipophilicity (C log P of 8.6), which limit action in the central nervous system.

Compounds that enhance the activity of M₁ muscarinic receptors in the central nervous system are useful in the treatment neurological disorders such as Alzheimer's disease, schizophrenia, and autism spectrum disorders. Compounds that enhance the ability of acetylcholine (ACh), the naturally occurring substance found in the brain, to activate M₁ muscarinic receptors through interaction with allosteric binding sites may be particularly useful in this manner. Such compounds do not directly activate M₁ muscarinic receptors, but enhance the responses produced by acetylcholine by binding to a different site on M₁ muscarinic receptors. Although many compounds have been identified that enhance the potency of acetylcholine (i.e., enhance binding cooperativity), relatively few compounds increase the activity of acetylcholine (i.e., enhance activation cooperativity) without directly activating M₁ muscarinic receptors. In accordance with the present disclosure, compounds which exhibit enhanced activation cooperativity without enhancing binding cooperativity have been synthesized. The compounds are useful in conditions where it is desirable to increase the overall activity of acetylcholine without changing the concentrations at which it activates receptors.

Positive allosteric modulators (PAMs) can exhibit three distinct properties: intrinsic allosteric agonist activity (PAM1), which is linked to adverse effects; allosteric modulation of potency (PAM2), which is the most common approach; or allosteric modulation of efficacy (PAM3), which is relatively unexplored. Compounds that enhance ACh efficacy provide unique ways to modify neuronal activity.

Selective enhancement of ACh efficacy may be particularly helpful in promoting encoding of new memories, which occurs at high ACh concentrations, and in restoring function to failed signaling pathways. The selective modulation of ACh potency and/or efficacy may be useful for the treatment of neurological disorders. Enhancement of responses produced by low concentrations of ACh (potency PAM) is associated with memory retrieval. Enhancement of responses produced by high concentrations of ACh (efficacy PAM) is associated with memory encoding, and may also restore functionality in a failed system. Different combinations may enhance potency and efficacy (super-PAM effect), resulting in increased memory retrieval and encoding, and signal shaping, having differential effects at low vs. high concentrations.

Provided herein are compounds which modulate the activity of muscarinic receptors. Previous positive allosteric modulators of muscarinic receptors act by enhancing ACh potency rather than efficacy, while the compounds herein exhibit unique properties as allosteric enhancers of ACh efficacy. In some embodiments, provided herein are allosteric modulators of M₁ muscarinic receptors which, as shown in the examples herein, enhance acetylcholine efficacy and decrease repetitive behaviors in zebrafish. The M₁ muscarinic receptor modulators may be useful in treating neurological disorders such as Alzheimer's disease, Parkinson's disease, schizophrenia, autism spectrum disorders, and post-traumatic stress disorder.

In general, the M₁ muscarinic receptor modulators are compounds having the following Formula I:

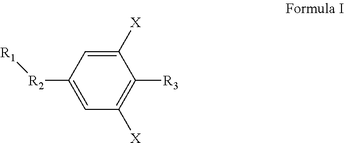

where each X is, independently, H, F, Cl, Br, I, OCH₃, CF₃, or OCF₃; R₁ is selected from

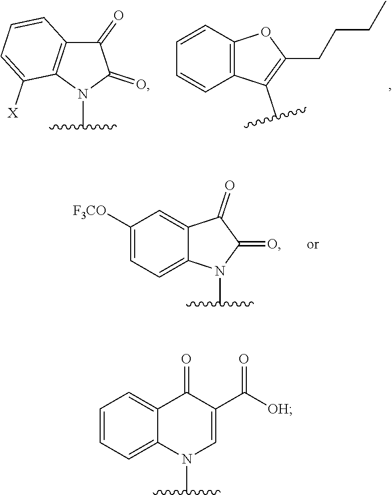

R₂ is selected from either

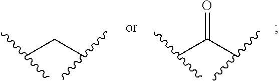

and R₃ is H, F, Br, OCF₃, OCH₃,

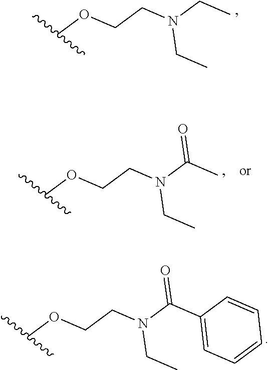

In some embodiments, the M₁ muscarinic receptor modulators are compounds having the following Formula II:

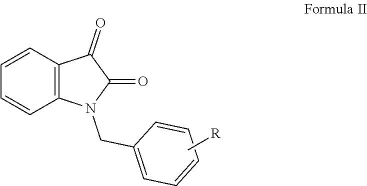

where R is an alkoxy or substituted alkoxy group, and optionally, the R-substituted phenyl ring is further substituted with one or more halogens.

In some embodiments, the M₁ muscarinic receptor modulators are compounds having the following Formula III:

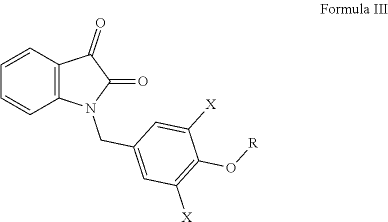

where each X is, independently, a halogen; and R is alkyl.

In some embodiments, the M₁ muscarinic receptor modulators are compounds having the following Formula IV:

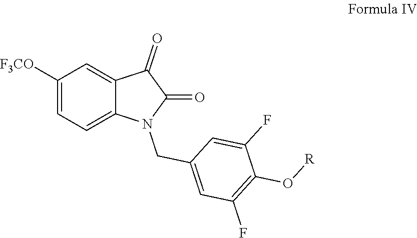

where each X is, independently, a halogen; and R is alkyl.

In some embodiments, the M₁ muscarinic receptor modulators are compounds having the following Formula V:

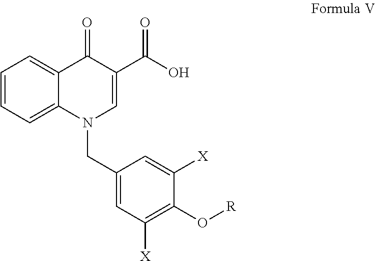

where each X is, independently, a halogen; and R is alkyl.

One non-limiting example M₁ muscarinic receptor modulator compound is 5a, which has the following structure:

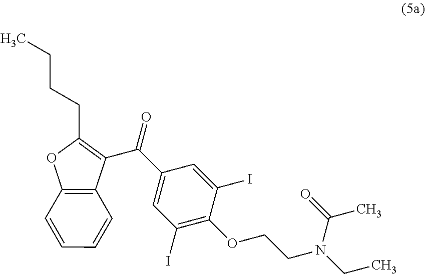 [SMILES: CCCCc1oc2ccccc2c1C(=O)c1cc(I)c(OCCN(CC)C(C)=O)c(I)c1]

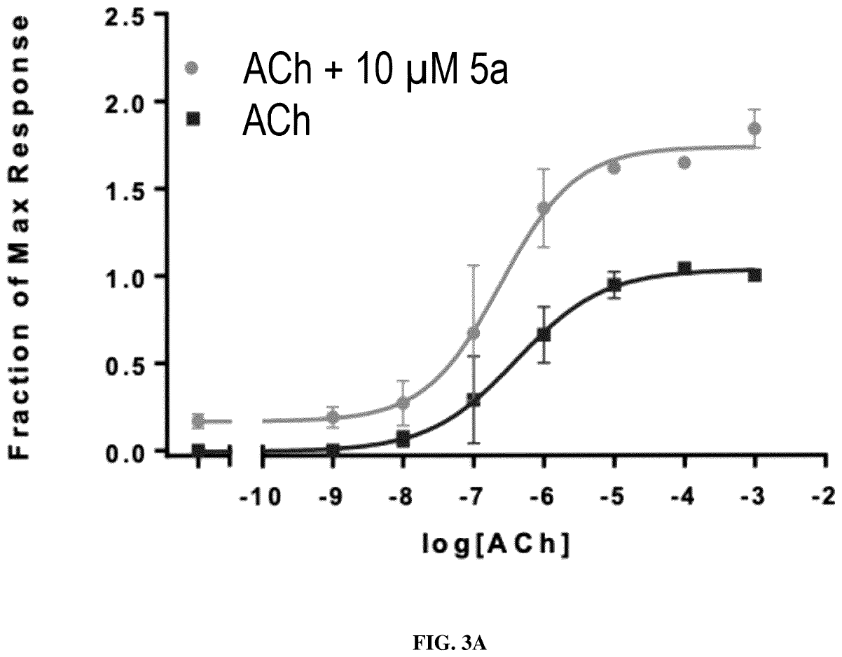

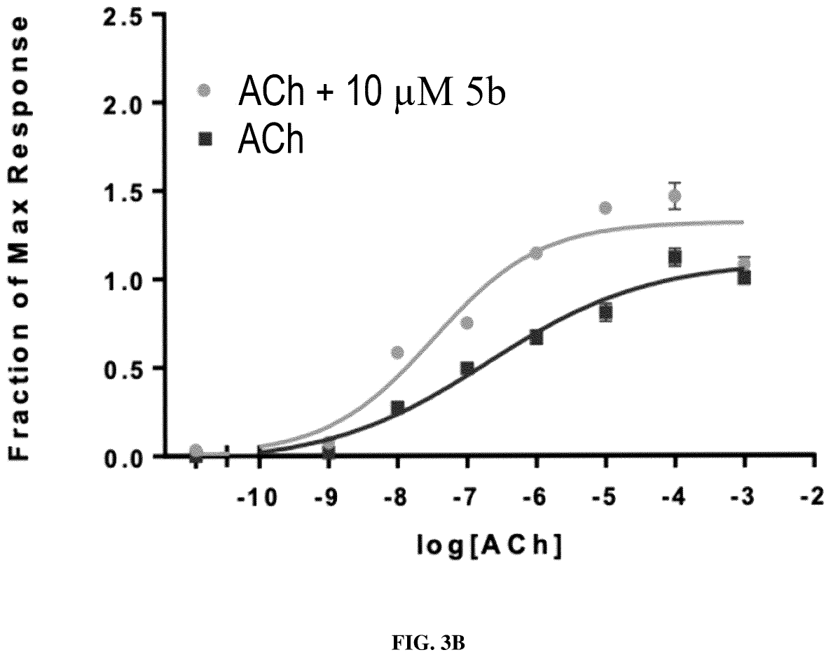

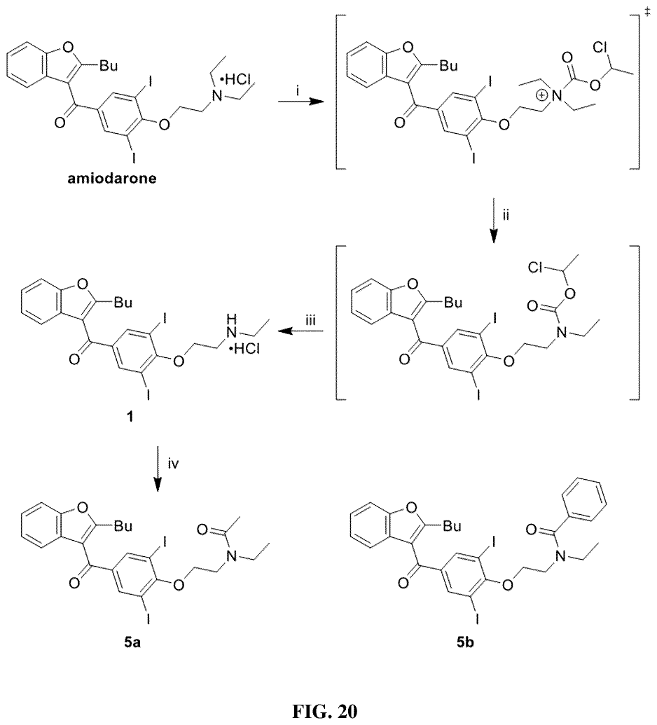

Compound 5a, also known as N-(2-(4-(2-butylbenzofuran-3-carbonyl)-2,6-diiodophenoxy)ethyl)-N-ethylacetanide, is an amide derivative of amiodarone that can be prepared according to the scheme depicted in FIG. 20. As shown in FIG. 20, a method of making a M₁ muscarinic receptor modulator compound such as 5a may include n-dealkylating amiodarone with a chloroethyl chloroformate to obtain a carbamate intermediate; allowing the carbamate intermediate to decompose into a decomposed intermediate; and reacting the decomposed intermediate with an anhydride to obtain a M₁ muscarinic receptor modulator compound. However, other methods of preparing 5a are possible and encompassed within the scope of the present disclosure. As shown in the examples herein, compound 5a enhances ACh efficacy at M₁ muscarinic receptors. (FIG. 3A.)

Another non-limiting example M₁ muscarinic receptor modulator compound is 5b, which has the following structure:

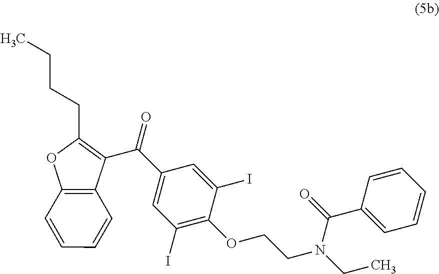 [SMILES: CCCCc1oc2ccccc2c1C(=O)c1cc(I)c(OCCN(CC)C(=O)c2ccccc2)c(I)c1]

Compound 5b, also known as N-(2-(4-(2-butylbenzofuran-3-carbonyl)-2,6-diiodophenoxy)ethyl)-N-ethylbenzamide, is another amide derivative of amiodarone that can also be prepared according to the scheme depicted in FIG. 20 and described above. However, other methods of preparing 5b are possible and encompassed within the scope of the present disclosure. Compound 5b also enhances ACh efficacy at M₁ muscarinic receptors. (FIG. 3B.)

Compounds 5a and 5b are allosteric modulators. However, amiodarone derivatives have a high molecular weight and lipophilicity, which may limit brain penetration.

Another non-limiting example M₁ muscarinic receptor modulator compound is compound 7, which has the following structure:

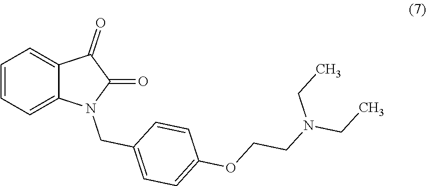 [SMILES: CCN(CC)CCOc1ccc(CN2C(=O)C(=O)c3ccccc32)cc1]

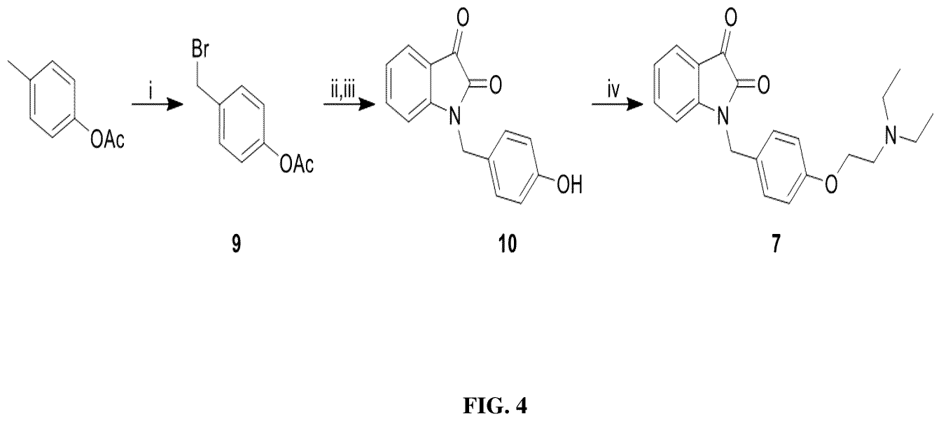

Compound 7, also known as 1-(4-(2-(diethylamino)ethoxy)benzyl)indoline-2,3-dione, can be prepared according to the scheme depicted in FIG. 4. As seen in FIG. 4, a method for making a M₁ muscarinic receptor modulator compound such as compound 7 may include brominating para-tolyl acetate with a brominating agent to obtain a benzyl bromide; alkylating the benzyl bromide with isatin to obtain an acetylated benzyl dione; deacetylating the acetylated benzyl dione to obtain a hydroxy benzyl dione; and subjecting the hydroxy benzyl dione to a Williamson ether synthesis with an alkyl halide to obtain the M₁ muscarinic receptor modulator compound. However, it is understood that other methods of synthesizing compound 7 are possible and encompassed within the scope of the present disclosure.

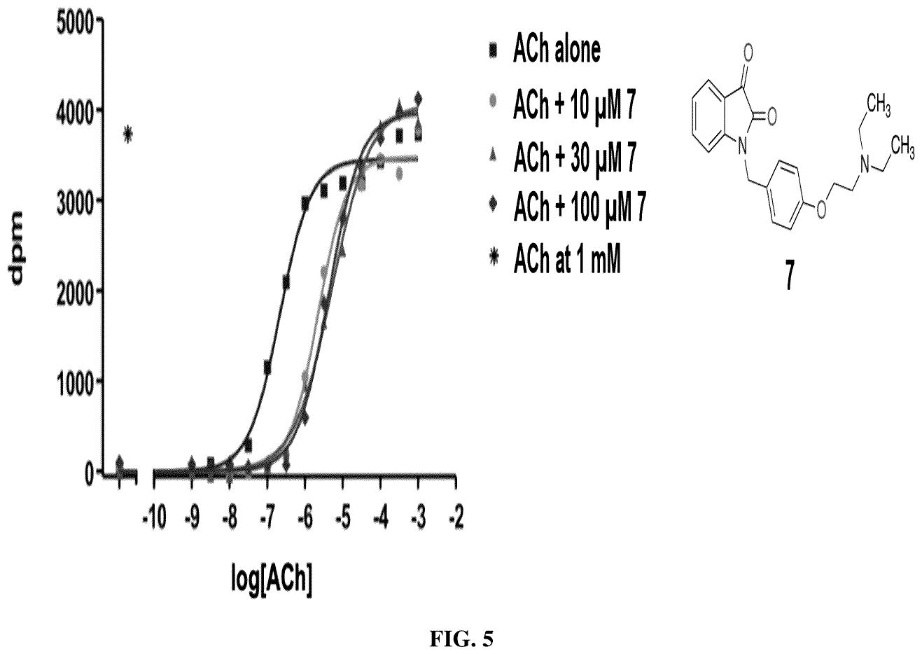

As seen in FIG. 5, compound 7 significantly enhances ACh efficacy, yet inhibits potency, at M₁ receptors expressed in CHO cells. As shown in FIG. 5, compound 7 produces a signal-sharpening effect with dose-dependent decreases in ACh potency and increases in ACh efficacy. The modulation of ACh activity produced by compound 7 indicates a signal sharpening effect, whereby activity is suppressed at low concentrations of ACh, yet enhanced at high concentrations of ACh. Compound 7 thus has the ability to shape neurotransmitter signals by suppressing activity of low ACh concentrations while enhancing activity at high ACh concentrations. Such activity may be particularly desirable in certain conditions, such as in post-traumatic stress disorder (PTSD), where recall of traumatic experiences results in increased anxiety. Compounds such as compound 7 decrease ACh potency, thereby suppressing the recall of memories associated with trauma (i.e., prevent memory retrieval) while enhancing ACh efficacy to permit encoding of new memories.

It should be noted that the impact of compound 7 on ACh potency is much more pronounced than the impact on ACh efficacy. The chemical structure can be modified to enhance the positive allosteric modulatory effects on efficacy while maintaining the negative allosteric modulatory effects on potency.

Another non-limiting example M₁ muscarinic receptor modulator compound is compound 8a, which has the following structure:

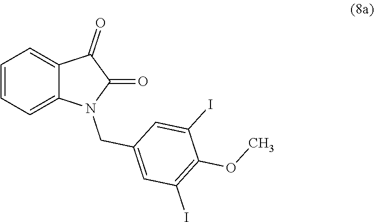 [SMILES: COc1c(I)cc(CN2C(=O)C(=O)c3ccccc32)cc1I]

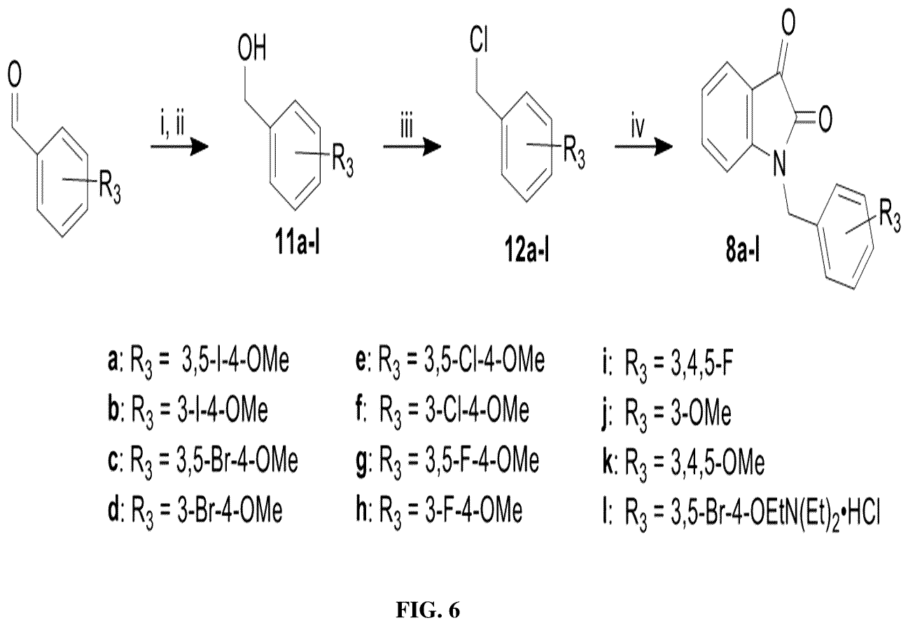

Compound 8a, also known as 1-(3,5-diiodo-4-methoxybenzyl)indoline-2,3-dione, can be prepared according to the scheme depicted in FIG. 6. As seen in FIG. 6, a method for making a M₁ muscarinic receptor modulator compound such as compound 8a may include alkylating a benzaldehyde with an unsubstituted phenol in the presence of a base to produce an alkylated benzaldehyde; reducing the alkylated benzaldehyde with a reducing agent to produce a benzyl alcohol; halogenating the benzyl alcohol with a halogenation agent to produce a benzyl halide; and N-alkylating isatin with the benzyl halide to obtain the M₁ muscarinic receptor modulator compound. However, it is understood that other methods of synthesizing compound 8a are possible and encompassed within the scope of the present disclosure.

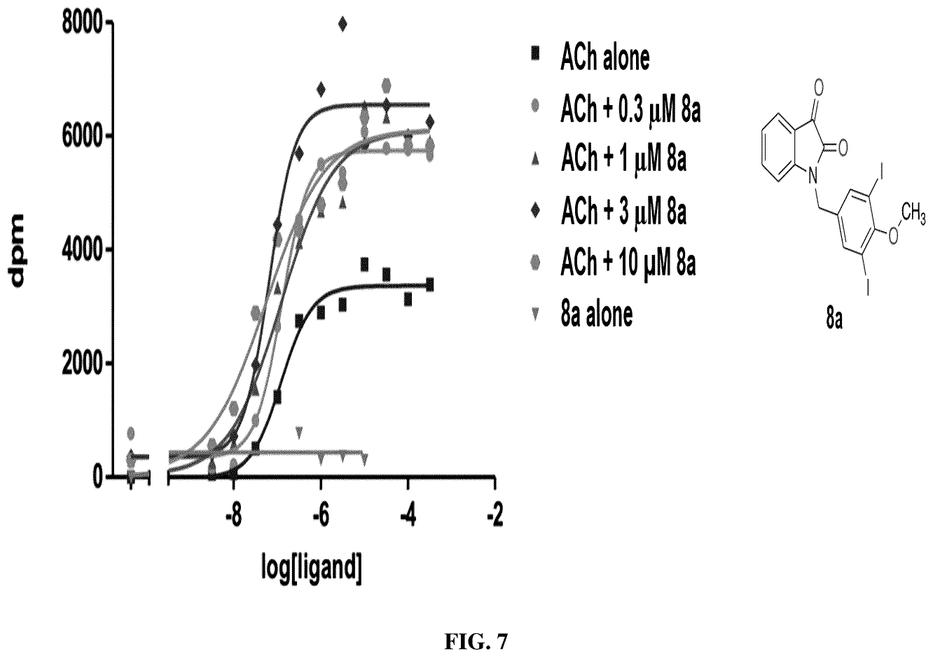

The effects of compound 8a on ACh responses are shown in FIG. 7. As seen in FIG. 7, compound 8a dramatically enhances ACh efficacy at M₁ receptors expressed in CHO cells. In contrast to the effects produced by compound 7 to modestly enhance ACh efficacy while dramatically inhibiting potency, compound 8a produces a dramatic increase in ACh efficacy with minimal effects on ACh potency. Moreover, compound 8a exhibits very low intrinsic activity as it does not directly promote the release of [3H]-AA. This profile may be particularly useful in restoring impaired muscarinic receptor signaling and enhancing memory encoding in neurological disorders impacting cholinergic pathways such as Alzheimer's disease. Enhancing ACh efficacy is useful in promoting encoding of memory function in Alzheimer's disease patients.

Another non-limiting example M₁ muscarinic receptor modulator compound is compound 8c, which has the following structure:

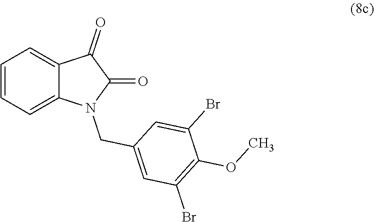 [SMILES: COc1c(Br)cc(CN2C(=O)C(=O)c3ccccc32)cc1Br]

Compound 8c, also known as 1-(3,5-dibromo-4-methoxybenzyl)indoline-2,3-dione, can be prepared according to the scheme depicted in FIG. 6 and described above. However, it is understood that other methods of synthesizing compound 8c are possible and encompassed within the scope of the present disclosure. Compound 8c is a direct analog of compound 8a incorporating bromines in place of the iodines within the benzyl ring.

Another non-limiting example M₁ muscarinic receptor modulator compound is compound 8j, which has the following structure:

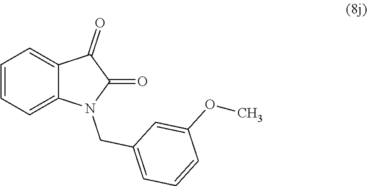 [SMILES: COc1cccc(CN2C(=O)C(=O)c3ccccc32)c1]

Compound 8j, also known as 1-(3-methoxybenzyl)indoline-2,3-dione, can be prepared according to the scheme depicted in FIG. 6 and described above. However, it is understood that other methods of synthesizing compound 8j are possible and encompassed within the scope of the present disclosure. 8j incorporates a methoxy group in the meta position of the benzyl group rather than the para position found in other compounds such as compound 7 and compound 8a.

Another non-limiting example M₁ muscarinic receptor modulator compound is compound GK-1-44, which has the following structure:

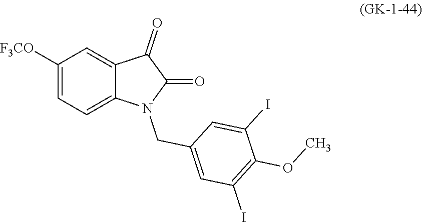 [SMILES: COc1c(I)cc(CN2C(=O)C(=O)c3cc(OC(F)(F)F)ccc32)cc1I]

Another non-limiting example M₁ muscarinic receptor modulator compound is compound GK-1-48, which has the following structure:

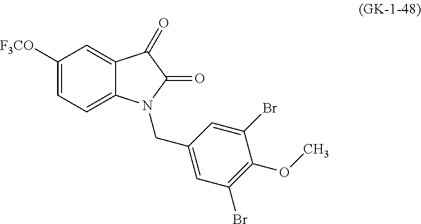 [SMILES: COc1c(Br)cc(CN2C(=O)C(=O)c3cc(OC(F)(F)F)ccc32)cc1Br]

Compounds GK-1-44 and GK-1-48 are 5-trifluoromethoxy-isatin derivatives. Compounds GK-1-44 and GK-1-48 can be prepared in a similar fashion to compounds 8a and 8c, depicted in the scheme shown in FIG. 6, except that 5-trifluoromethoxy-isatin is used in step iv in place of isatin.

Another non-limiting example M₁ muscarinic receptor modulator compound is compound GK-1-47, which has the following structure:

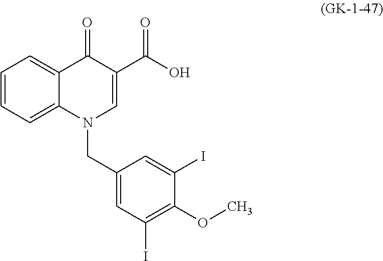 [SMILES: COc1c(I)cc(Cn2cc(C(=O)O)c(=O)c3ccccc32)cc1I]

Another non-limiting example M₁ muscarinic receptor modulator compound is compound GK-1-50, which has the following structure:

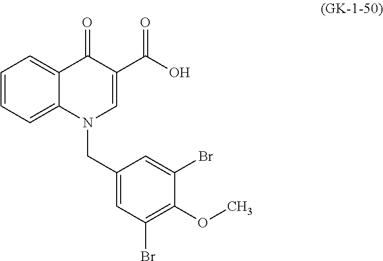 [SMILES: COc1c(Br)cc(Cn2cc(C(=O)O)c(=O)c3ccccc32)cc1Br]

Compounds GK-1-47 and GK-1-50 are benzoquinolone carboxylic acid derivatives. Compounds GK-1-47 and GK-1-50 can be prepared in a similar fashion to the N-benzyl isatin analogues (e.g., compounds 8a and 8c, as depicted in FIG. 6) with an additional ester hydrolysis finishing step. Compounds GK-1-47 and GK-1-50 are M₁ selective.

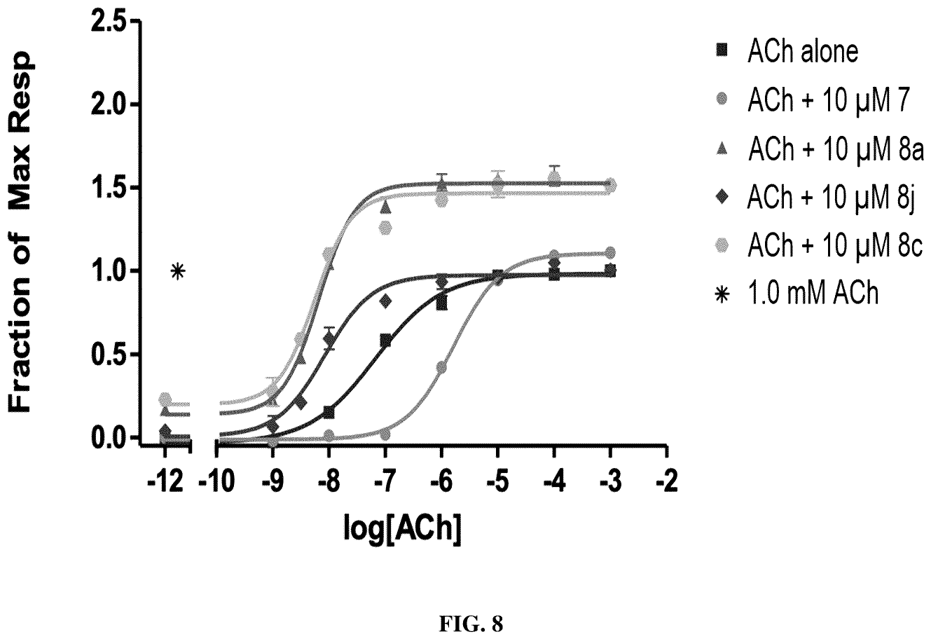

FIG. 8 shows a summary of, and compares, the effects of the allosteric modulators 7, 8a, 8j, and 8c (each at a concentration of 10 μM) on the stimulation of [3H]-arachidonic acid release by acetylcholine (i.e., ACh activity) at M₁ muscarinic receptors expressed in CHO cells. Both 8a and 8c (di-halogenated compounds) enhance ACh efficacy, while the meta-methoxy derivative 8j enhances ACh potency. In contrast, 7 decreases ACh potency while modestly enhancing ACh efficacy. Compounds GK-1-44, GK-1-48, GK-1-47, and GK-1-50 also enhance the effects of 100 μM acetylcholine (ACh), and compound GK-1-48 produces a significant decrease in repetitive behaviors, as shown in the examples herein. Thus, the compounds exhibit markedly different and distinct allosteric modulatory profiles at M₁ muscarinic receptors.

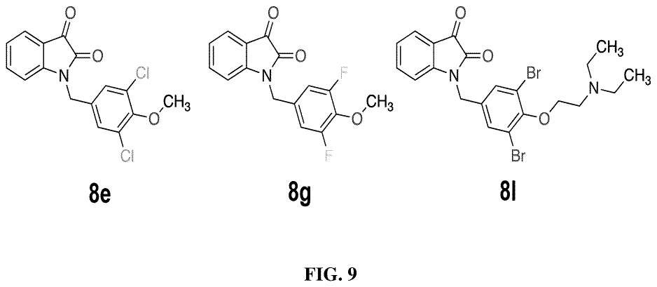

The effects of substitution with other halogens (chlorine and fluorine) and combinations of halogenation with a tertiary amine have been further explored. Some non-limiting examples of such compounds that have been synthesized (namely, 8e, 8g, and 8l) are shown in FIG. 9. Accordingly, another non-limiting example M₁ muscarinic receptor modulator compound is compound 8l, which has the following structure:

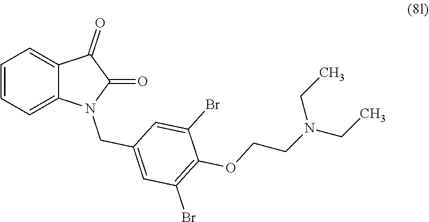 [SMILES: CCN(CC)CCOc1c(Br)cc(CN2C(=O)C(=O)c3ccccc32)cc1Br]

Compound 8l, also known as 1-(3,5-dibromo-4-(2-(diethylamino)ethoxy)benzyl)indoline-2,3-dione hydrochloride, can be prepared according to the scheme depicted in FIG. 6 and described above. However, it is understood that other methods of synthesizing compound 8l are possible and encompassed within the scope of the present disclosure.

Another non-limiting example M₁ muscarinic receptor modulator compound is compound 8e, which has the following structure:

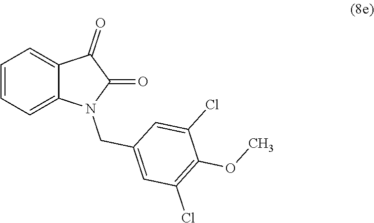 [SMILES: COc1c(Cl)cc(CN2C(=O)C(=O)c3ccccc32)cc1Cl]

Compound 8e, also known as 1-(3,5-dichloro-4-methoxybenzyl)indoline-2,3-dione, can be prepared according to the scheme depicted in FIG. 6 and described above. However, it is understood that other methods of synthesizing compound 8e are possible and encompassed within the scope of the present disclosure.

Another non-limiting example M₁ muscarinic receptor modulator compound is compound 8g, which has the following structure:

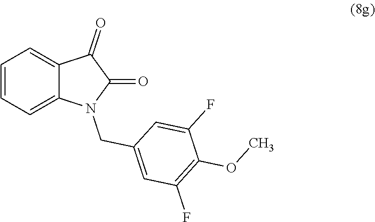 [SMILES: COc1c(F)cc(CN2C(=O)C(=O)c3ccccc32)cc1F]

Compound 8g, also known as 1-(3,5-difluoro-4-methoxybenzyl)indoline-2,3-dione, can be prepared according to the scheme depicted in FIG. 6 and described above. However, it is understood that other methods of synthesizing compound 8g are possible and encompassed within the scope of the present disclosure.

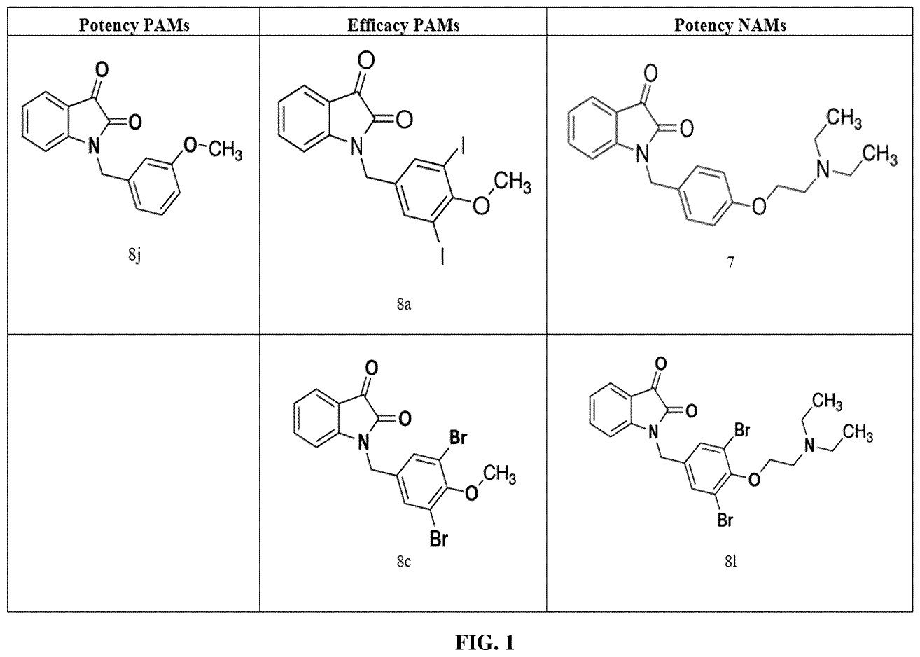

The chart in FIG. 1 depicts some of the M₁ muscarinic modulator compounds with respect to being potency PAMs, efficacy PAMs, or potency PAMs.

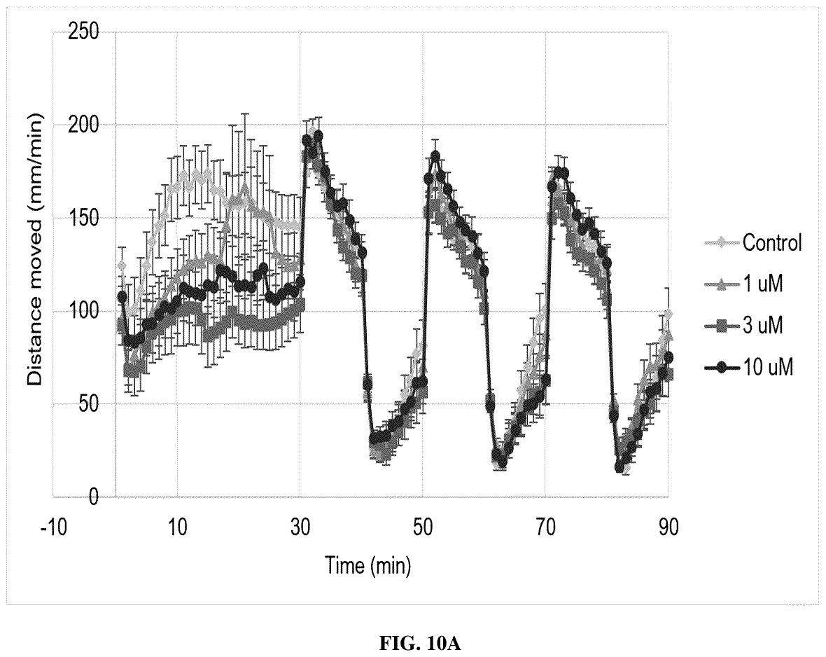

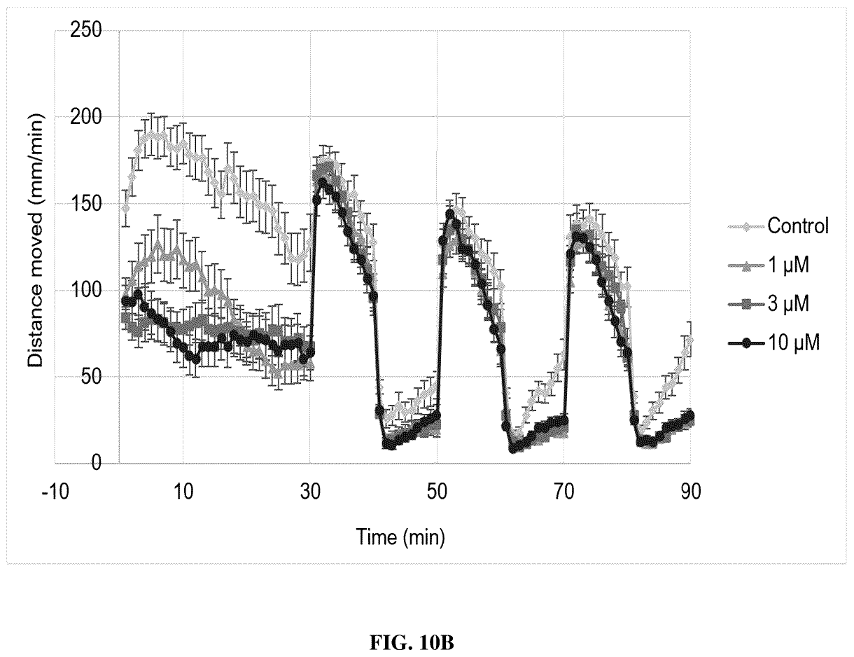

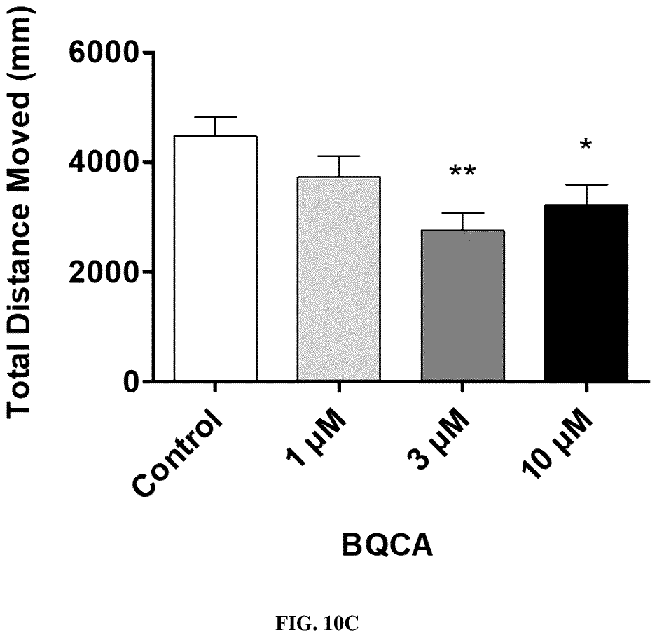

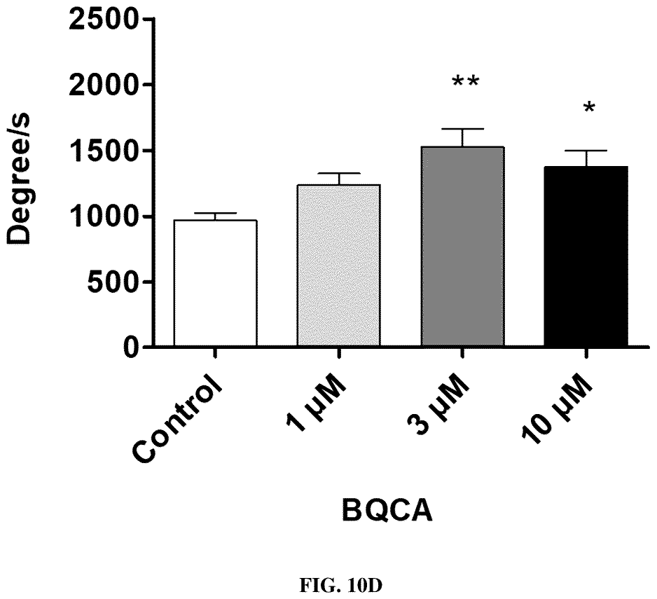

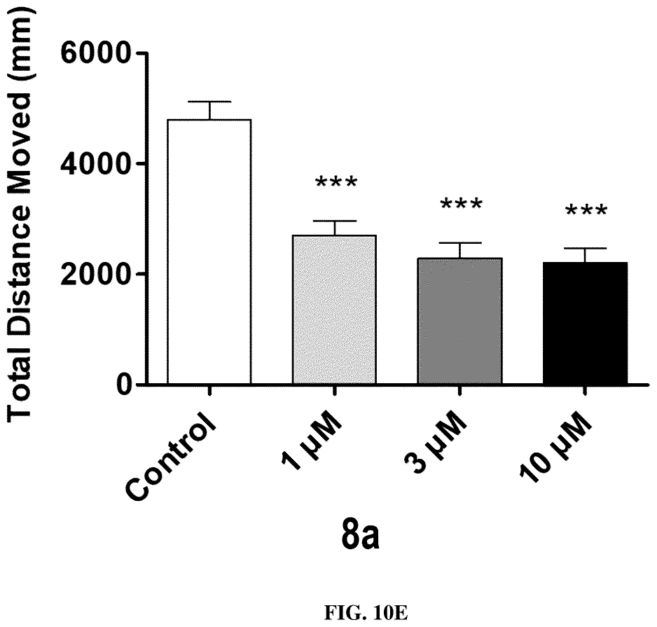

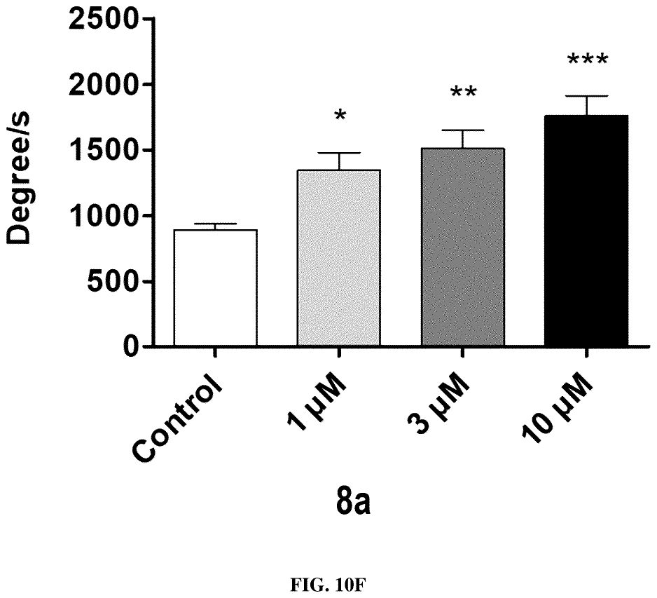

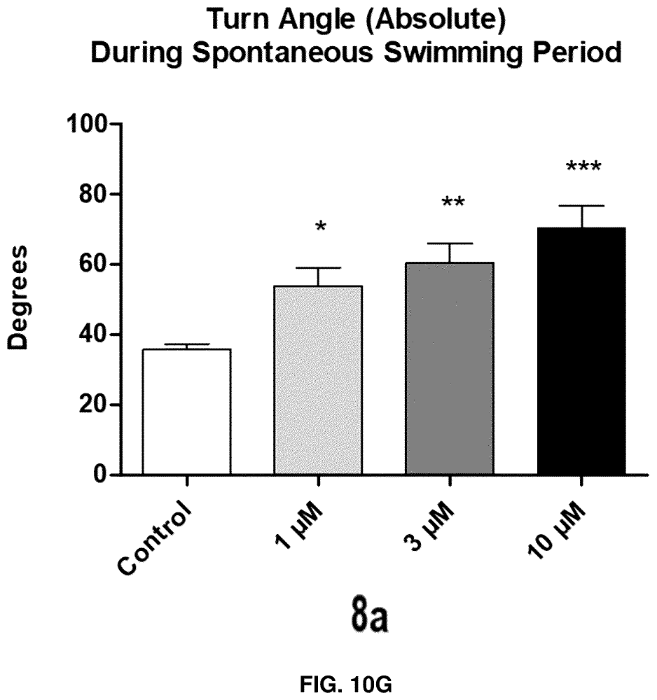

The allosteric modulation of efficacy is particularly advantageous because it may be useful in enhancing memory consolidation and restoring function in failed systems (e.g., Alzheimer's disease). Positive allosteric modulation of M₁ muscarinic receptors provides an alternative approach for the treatment of, for instance, autism spectrum disorder symptoms. In the examples herein, several allosteric modulators were tested for effects on repetitive behaviors in wild-type zebrafish. The compounds were assessed in vivo in zebrafish to determine 1) the maximum tolerated concentration as a measure of toxicity, and 2) the effects on locomotor behaviors, with a particular emphasis on repetitive behaviors. Zebrafish provide a useful screen for repetitive behaviors. Zebrafish strains are available with mutations in autism spectrum disorder-related genes (e.g., Shank3 deletions). In the examples herein, the behavioral effects of the compounds in wild-type zebrafish were assessed by analyzing repetitive behaviors (turn angle, anguar velocity), and locomotor activity. The results showed that M₁ receptor efficacy-based PAMs decrease repetitive behaviors as measured by angular velocity and turn angle. (FIGS. 10A-10G.)

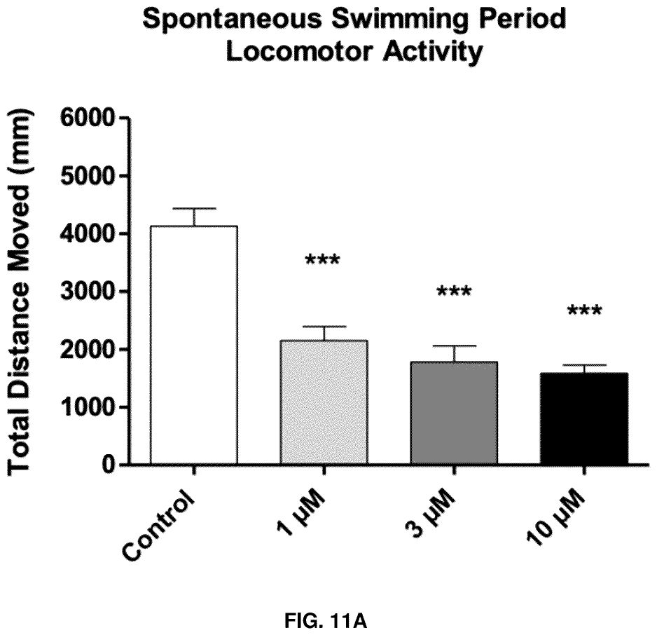

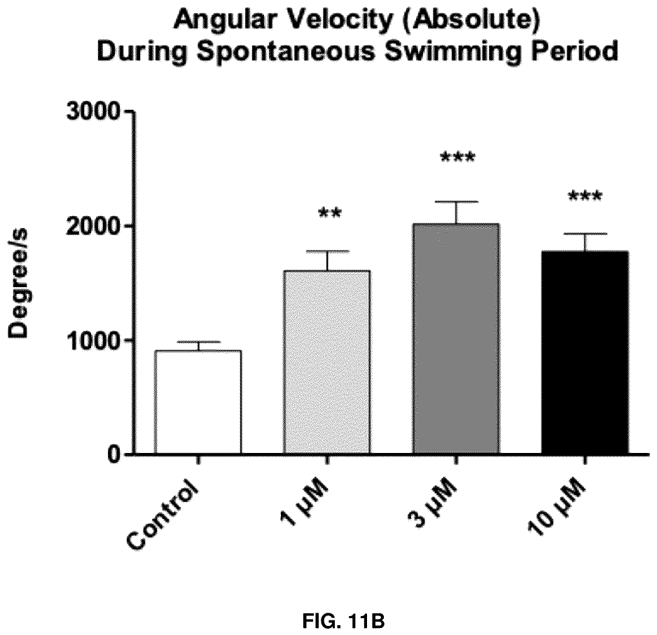

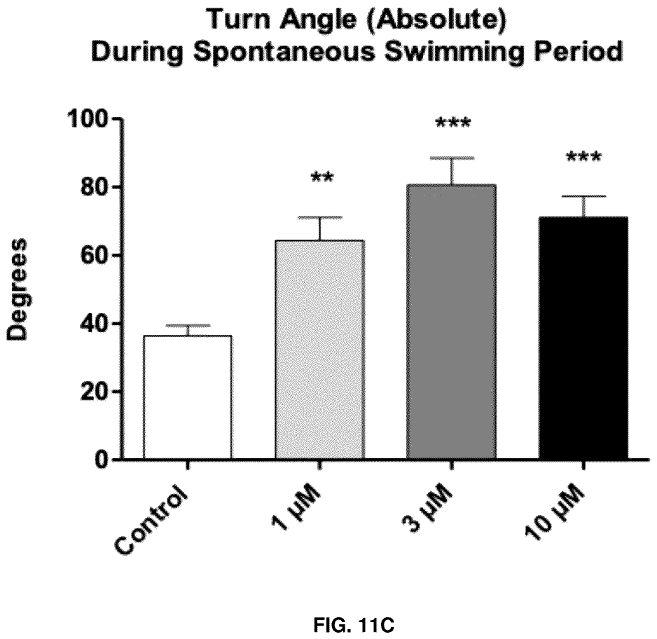

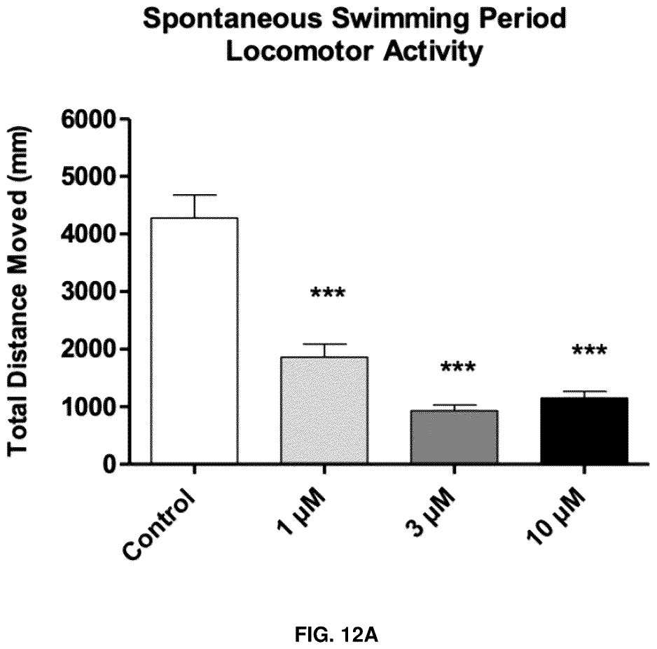

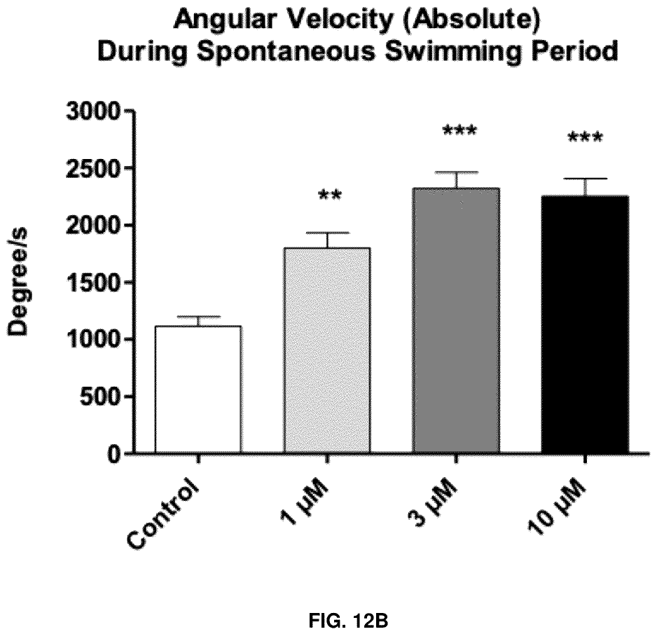

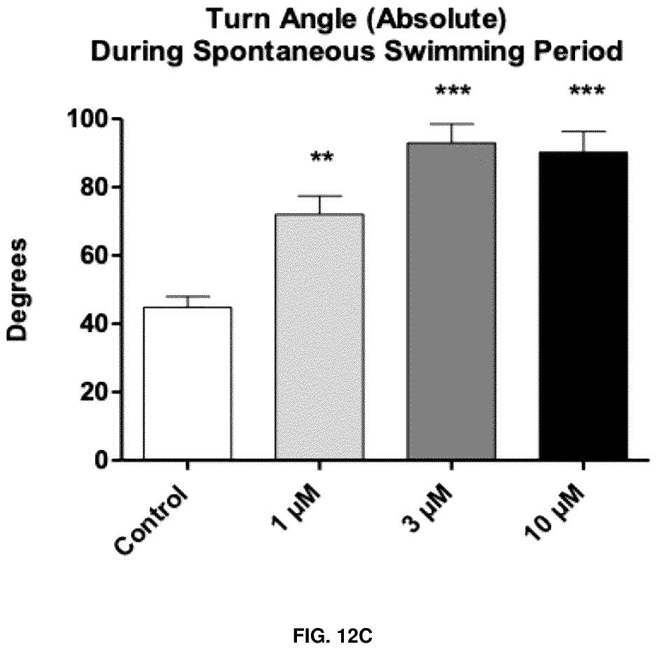

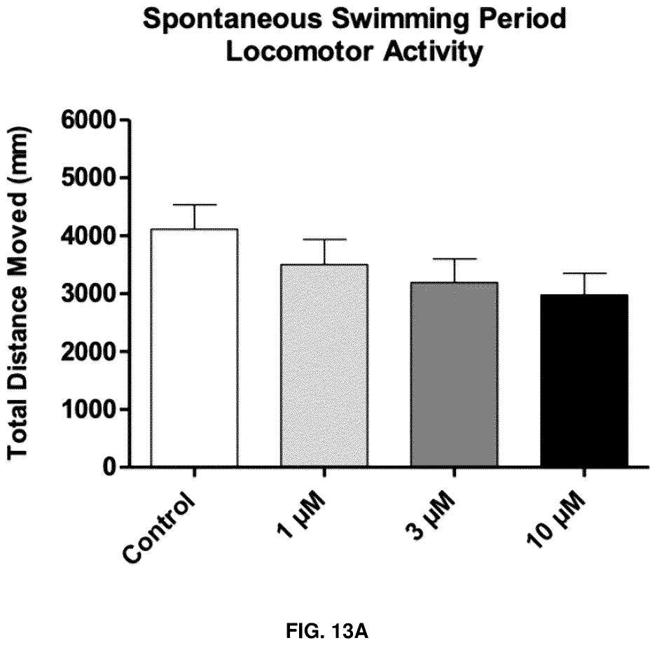

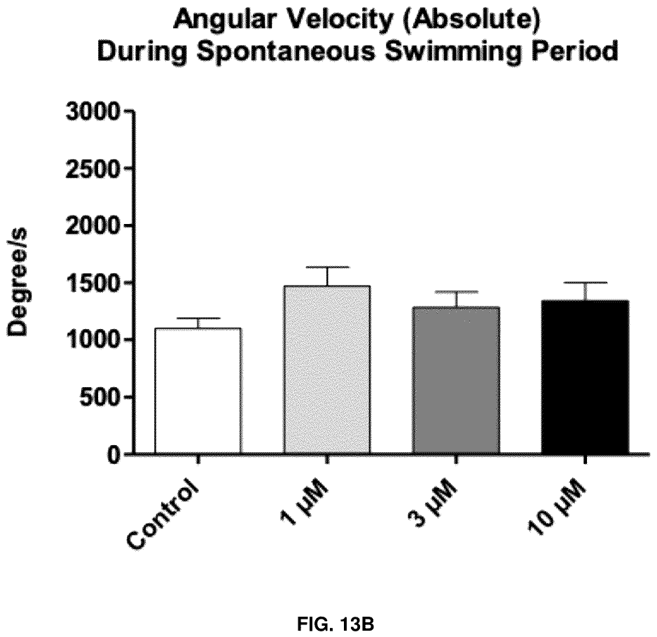

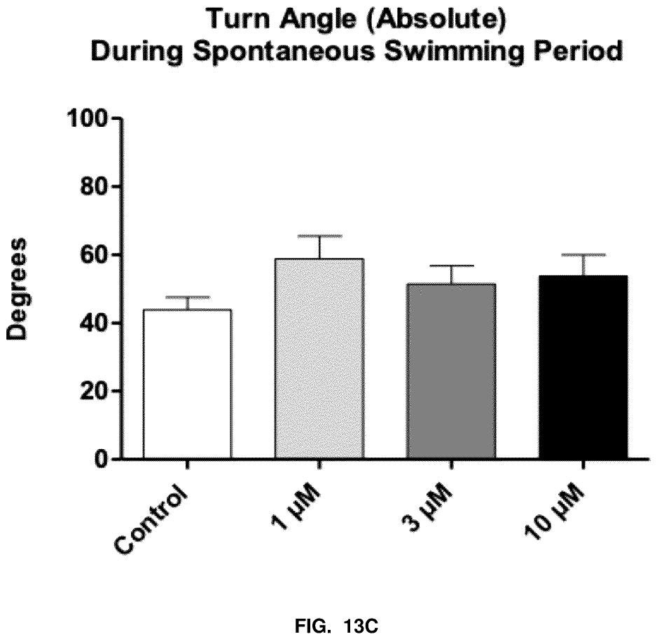

As shown in FIGS. 11A-11C, 8j significantly decreased locomotor activity and increased angular velocity and absolute turn angle. The latter two observations indicate that 8j decreases repetitive behaviors. As seen in FIGS. 10F-10G, the M₁ receptor-based PAM 8a decreases repetitive behaviors as measured by angular velocity and turn around. As shown in FIGS. 10E-10G, 8a also decreased activity in both light and dark periods, significantly decreased locomotor activity in spontaneous swimming period, and increased angular velocity and absolute turn angle. The latter two observations indicate that compound 8a decreases repetitive behaviors. Similar effects were observed with compound 8c, as shown in FIGS. 12A-12C. Compound 8c also significantly decreased locomotor activity and increased angular velocity and absolute turn angle. The latter two observations indicate that compound 8c decreases repetitive behaviors. In contrast to the effects of compounds 8j, 8a, and 8c on locomotor behaviors, compound 7 did not significantly alter locomotor activity, angular velocity, and turn angle in zebrafish (see FIGS. 13A-13C). These data indicate that enhancement of ACh potency is important for reduced locomotor activity and decreases in repetitive behaviors (as reflected by increases in angular velocity and turn angle).

The data from the zebrafish in the examples herein indicate that the M₁ muscarinic receptor modulator compounds are able to penetrate into the brain and reduce repetitive behaviors. Compounds that alleviate restrictive and repetitive behaviors may help promote cognitive function and increase socialization. Accordingly, this shows that the M₁ muscarinic receptor modulator compounds herein may be useful in treating a variety of neurological disorders including, but not limited to, Alzheimer's disease, autism spectrum disorders, schizophrenia, post-traumatic stress disorder (PTSD), and substance abuse.

The compounds described herein can be used and administered in different types of simple and complex formulations. They can be formulated simply by dissolution in different solutions. Alternatively, they can be formulated as complex pharmaceutical delivery systems such as tablets, capsules, powders, with a range of reagents, excipients, stabilizers, and the like.

Pharmaceutical compositions of the present disclosure may comprise an effective amount of a muscarinic receptor modulator compound described herein (an “active ingredient”), and/or additional agents, dissolved or dispersed in a pharmaceutically acceptable carrier. The preparation of a pharmaceutical composition will be known to those of skill in the art in light of the present disclosure, as exemplified by Remington's Pharmaceutical Sciences, 2003, incorporated herein by reference. Moreover, for animal (e.g., human) administration, it is understood that preparations should meet sterility, pyrogenicity, general safety, and purity standards as required by FDA Office of Biological Standards.

A composition disclosed herein may comprise different types of carriers depending on whether it is to be administered in solid, liquid or aerosol form, and whether it need to be sterile for such routes of administration as injection. Compositions disclosed herein can be administered intravenously, intradermally, transdermally, intrathecally, intraarterially, intraperitoneally, intranasally, intravaginally, intrarectally, intraosseously, periprosthetically, topically, intramuscularly, subcutaneously, mucosally, intraosseosly, periprosthetically, in utero, orally, topically, locally, via inhalation (e.g., aerosol inhalation), by injection, by infusion, by continuous infusion, by localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 2003, incorporated herein by reference).

The actual dosage amount of a composition disclosed herein administered to an animal or human patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In certain embodiments, a composition herein and/or additional agent is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsules, they may be compressed into tablets, or they may be incorporated directly with the food of the diet.

In further embodiments, a composition described herein may be administered via a parenteral route. As used herein, the term “parenteral” includes routes that bypass the alimentary tract. Specifically, the pharmaceutical compositions disclosed herein may be administered, for example but not limited to, intravenously, intradermally, intramuscularly, intraarterially, intrathecally, subcutaneous, or intraperitoneally (U.S. Pat. Nos. 6,753,514, 6,613,308, 5,466,468, 5,543,158; 5,641,515, and 5,399,363 are each specifically incorporated herein by reference in their entirety).

Solutions of the compositions disclosed may be prepared in water suitably mixed with a surfactant, such as hydroxypropylcellulose. Dispersions may also be prepared in glycerol, liquid polyethylene glycols and mixtures thereof, and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In some cases, the form should be sterile and should be fluid to the extent that easy injectability exists. It should be stable under the conditions of manufacture and storage and should be preserved against the contaminating action of microorganisms, such as bacteria and fungi. The carrier can be a solvent or dispersion medium containing, for example, water, ethanol, polyol (i.e., glycerol, propylene glycol, liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be brought about by various antibacterial and antifungal agents, such as, but not limited to, parabens, chlorobutanol, phenol, sorbic acid, thimerosal, and the like. In some cases, it may be preferable to include isotonic agents, for example, sugars or sodium chloride. Prolonged absorption of the injectable compositions can be brought about by the use in the compositions of agents delaying absorption such as, for example, aluminum monostearate or gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

Sterile injectable solutions are prepared by incorporating the compositions in the required amount in the appropriate solvent with various other ingredients enumerated above, as required, followed by filtered sterilization. Generally, dispersions are prepared by incorporating the various sterilized compositions into a sterile vehicle which contains the basic dispersion medium and the required other ingredients from those enumerated above. In the case of sterile powders for the preparation of sterile injectable solutions, some methods of preparation are vacuum-drying and freeze-drying techniques which yield a powder of the active ingredient plus any additional desired ingredient from a previously sterile-filtered solution thereof. A powdered composition is combined with a liquid carrier such as, but not limited to, water or a saline solution, with or without a stabilizing agent.

In other embodiments, the compositions may be formulated for administration via various miscellaneous routes, for example, topical (i.e., transdermal) administration, mucosal administration (intranasal, vaginal, etc.), and/or via inhalation.

Pharmaceutical compositions for topical administration may include the compositions formulated for a medicated application such as an ointment, paste, cream, or powder. Ointments include all oleaginous, adsorption, emulsion, and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones, and luarocapram. Possible bases for compositions for topical application include polyethylene glycol, lanolin, cold cream, and petrolatum, as well as any other suitable absorption, emulsion, or water-soluble ointment base. Topical preparations may also include emulsifiers, gelling agents, and antimicrobial preservatives as necessary to preserve the composition and provide for a homogenous mixture. Transdermal administration of the compositions may also comprise the use of a “patch.” For example, the patch may supply one or more compositions at a predetermined rate and in a continuous manner over a fixed period of time.

In certain embodiments, the compositions may be delivered by eye drops, intranasal sprays, inhalation, and/or other aerosol delivery vehicles. Methods for delivering compositions directly to the lungs via nasal aerosol sprays has been described in U.S. Pat. Nos. 5,756,353 and 5,804,212 (each specifically incorporated herein by reference in their entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts and could be employed to deliver the compositions described herein. Likewise, transmucosal drug delivery in the form of a polytetrafluoroetheylene support matrix is described in U.S. Pat. No. 5,780,045 (specifically incorporated herein by reference in its entirety), and could be employed to deliver the compositions described herein.

It is further envisioned the compositions disclosed herein may be delivered via an aerosol. The term aerosol refers to a colloidal system of finely divided solid or liquid particles dispersed in a liquefied or pressurized gas propellant. The typical aerosol for inhalation consists of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight, and the severity and response of the symptoms.

In particular embodiments, the compounds and compositions described herein are useful for treating various neurological disorders such as, but not limited to, Alzheimer's disease, Parkinson's disease, autism spectrum disorders, substance abuse, and post-traumatic stress disorder. The compounds and compositions herein can be used in combination therapies. That is, the compounds and compositions can be administered concurrently with, prior to, or subsequent to one or more other desired therapeutic or medical procedures or drugs. The particular combination of therapies and procedures in the combination regimen will take into account compatibility of the therapies and/or procedures and the desired therapeutic effect to be achieved. Combination therapies include sequential, simultaneous, and separate administration of the active compound in a way that the therapeutic effects of the first administered procedure or drug are not entirely disappeared when the subsequent procedure or drug is administered.

By way of a non-limiting example of a combination therapy, the compounds or compositions described herein can be administered in combination with one or more cholinesterase inhibitors such as galantamine, rivastigmine, or donepezil. As another non-limiting example of a combination therapy, the compounds or compositions described herein can be administered in combination with one or more selective serotonin reuptake inhibitors (SSRIs) such as paroxetine, fluoxetine, or sertraline. The compounds and compositions described herein can also be combined with one or more of cognitive therapy, exposure therapy, eye movement densitization and reprocessing (EMDR), joint attention therapy, nutritional therapy, occupational therapy, or behavioral management therapy. Many other combination therapies are possible and encompassed within the scope of the present disclosure.

Unless stereochemistry is specifically indicated, all stereoisomers of the compounds herein are included, as pure compounds as well as mixtures thereof. It will be appreciated by one of ordinary skill in the art that asymmetric centers may exist in any of the compounds disclosed herein. Thus, the compounds and compositions thereof may be in the form of an individual enantiomer, diastereomer, or geometric isomer, or may be in the form of a mixture of stereoisomers. In certain embodiments, the compounds herein are enantiopure compounds. In certain other embodiments, mixtures of stereoisomers or diastereomers are provided. Additionally, the compounds encompass both (Z) and (E) double bond isomers (or cis and trans isomers) unless otherwise specifically designated. Thus, compounds generally depicted in structures herein encompass those structures in which double bonds are (Z) or (E).

The term “composition” as used herein is intended to encompass a product comprising the specified ingredients in the specified amounts, as well as any product that results, directly or indirectly, from combinations of the specified ingredients in the specified amounts.

The term “solvate” refers to a pharmaceutically acceptable solid form of a specified compound containing solvent molecules as part of the crystal structure. A solvate typically retains at least some of the biological effectiveness of such compound. Solvates can have different solubilities, hygroscopicities, stabilities, and other properties. Examples of solvates include, but are not limited to, compounds in combination with water, isopropanol, ethanol, methanol, DMSO, ethyl acetate, acetic acid, or ethanolamine. Solvates are sometimes termed “pseudopolymorphs.”

The term “hydrate” refers to a solvate with water.

The term “racemate” refers to a mixture that contains an equal amount of enantiomers.

The term “polymorph” means a crystalline form of a substance that is distinct from another crystalline form of the substance but that shares the same chemical formula.

It will be appreciated that any of the compounds described herein may be substituted with any number of substituents or functional moieties. In general, the term “substituted,” whether preceded by the term “optionally” or not, and substituents contained in formulas, refer to the replacement of hydrogen atoms in a given structure with a specified substituent. When more than one position in any given structure may be substituted with more than one substituent selected from a specified group, the substituent may be either the same or different at every position. The term “substituted” is contemplated to include all permissible substituents of organic compounds. In a broad aspect, the permissible substituents include acyclic and cyclic, branched and unbranched, carbocyclic and heterocyclic, aromatic and nonaromatic substituents or organic compounds. For purposes of explanation herein, heteroatoms such as nitrogen may have hydrogen substituents and/or any permissible substituents of organic compounds described herein which satisfy the valencies of the heteroatoms. Furthermore, there is not any intention to be limited in any manner by the permissible substituents of organic compounds. Combinations of substituents and variables envisioned are, in some embodiments, those that result in the formation of stable compounds useful in the treatment, for example, of neurological disorders.

The term “pharmaceutically acceptable carrier” means a medium that is used to prepare a desired dosage form of a compound. A pharmaceutically acceptable carrier includes solvents, diluents, or other liquid vehicles; dispersion or suspension aids; surface active agents; isotonic agents; thickening or emulsifying agents; preservatives; solid binders; lubricants; and the like.

The term “alkyl” refers to monovalent alkyl groups having from 1 to 50 carbon atoms, preferably having from 1 to 10 carbon atoms, and more preferably having from 1 to 6 carbon atoms. This term is exemplified by groups such as methyl, ethyl, n-propyl, iso-propyl, n-butyl, iso-butyl, n-hexyl, and the like. “Substituted alkyl” refers to an alkyl group, preferably of from 1 to 10 carbon atoms, having from 1 to 3 substituents selected from the group consisting of alkoxy, substituted alkoxy, acyl, acylamino, amino, aminoacyl, aminocarboxy esters, cyano, cycloalkyl, halogen, hydroxyl, carboxyl, carboxylalkyl, oxyacyl, oxyacylamino, thiol, thioalkoxy, substituted thioalkoxy, aryl, heteroaryl, heterocyclic, aryloxy, thioaryloxy, heteroaryloxy, thioheteroaryloxy, nitro, and mono- and di-alkylamino, mono- and di-(substituted alkyl)amino, mono- and di-arylamino, mono- and di-heteroarylamino, mono- and di-heterocyclic amino, and unsymmetric di-substituted amines having different substituents selected from alkyl, substituted alkyl, aryl, heteroaryl, and heterocyclic.

The term “alkoxy” refers to the group R—O, where R is alkyl. This term is exemplified by groups such as methoxy, ethoxy, propoxy, isopropoxy, cyclopropyloxy, cyclohexyloxy, and the like. “Substituted alkoxy” refers to alkoxy wherein the alkyl constituent is substituted with one or more substituents such as, but not limited to, alkyl, heteroalkyl, alkenyl, alkynyl, cycloalkyl, heterocycloalkyl, aryl, arylalyl, heteroaryl, halo, cyano, trifluoromethyl, trifluoromethoxy, nitro, trimethylsilanyl, or hydroxy.

### EXAMPLES

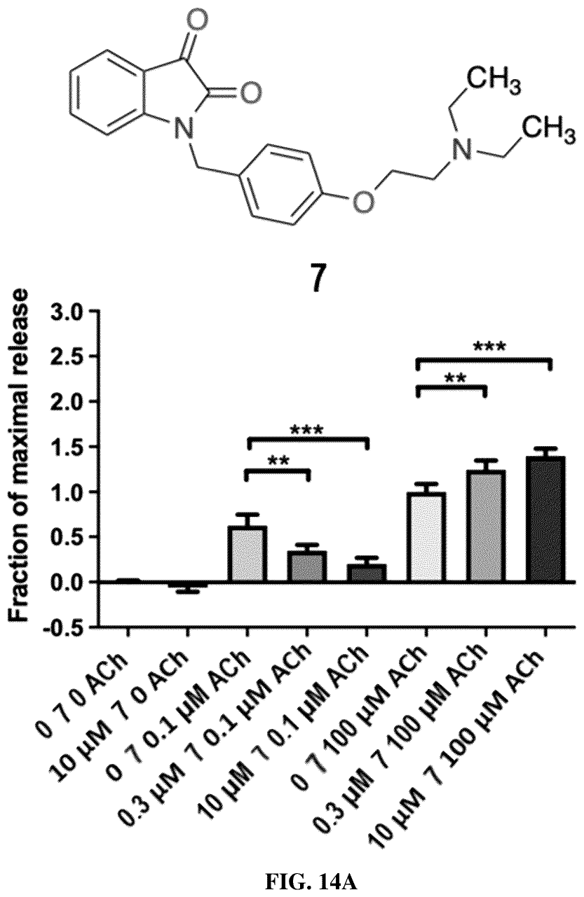

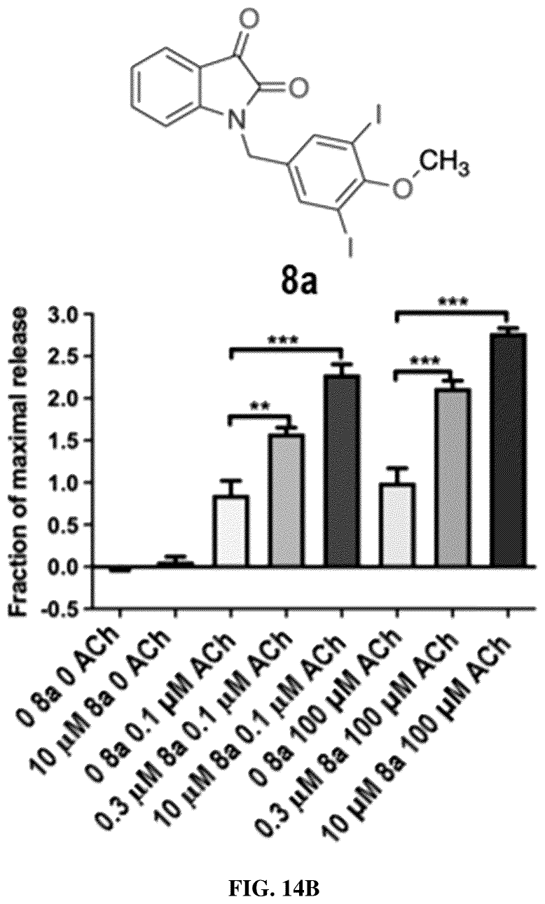

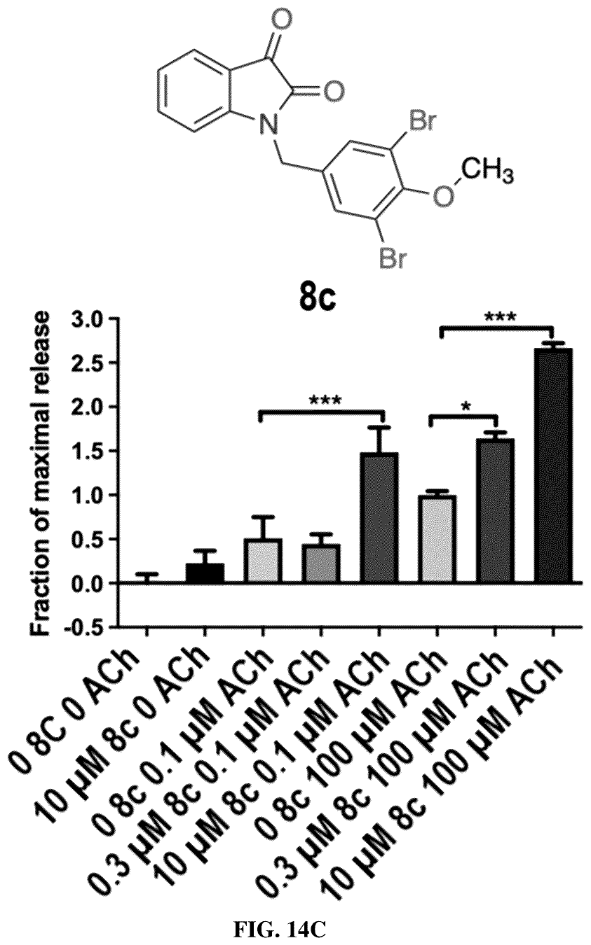

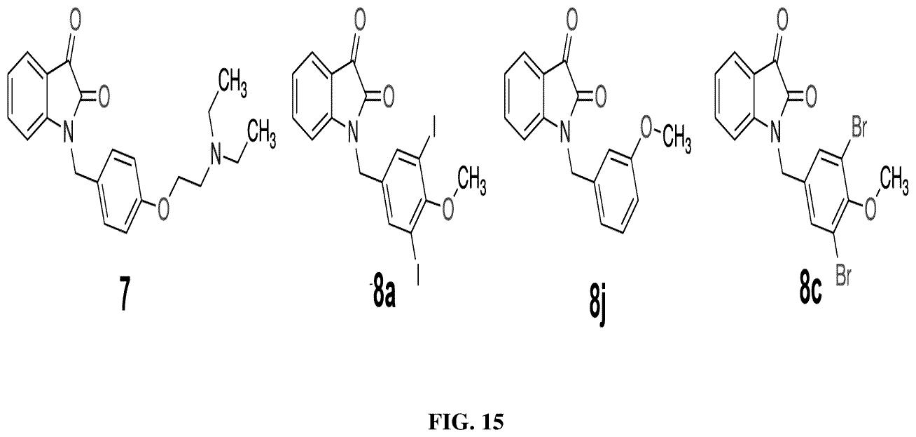

Allosteric modulation of muscarinic acetylcholine receptors (mAChRs) is a viable strategy for regulating cholinergic signaling in the treatment of various neurological disorders. Most positive allosteric modulators (PAMs) of mAChRs have been demonstrated to enhance agonist affinity, with few examples known of PAMs that selectively enhance G-protein coupling efficacy (e.g., amiodarone). In these examples, the key structural features of amiodarone responsible for enhancement of mAChR efficacy assayed in M₁ expressing CHO cells were identified. Subsequent incorporation of these structural features into n-benzyl isatins (allosteric modulators of potency) generated hybrid ligands that demonstrated similar or better enhancement of mAChR efficacy, less toxicity, and higher allosteric binding affinity relative to amiodarone. Several isatin analogs were synthesized and assessed for activity in screens for modulation of ACh activity (FIGS. 14A-14C), and then tested for activity using full ACh dose response curves. Notable hybrid ligands include 8a and 8c, which respectively demonstrated the strongest binding affinity and the most robust enhancement of mAChR efficacy as calculated from the allosteric operational model. Based on the promising effects of 8a on ACh efficacy, other isatin derivatives were synthesized, including 8j and 8c (see FIG. 15). Amiodarone derivatives and hybrid ligands were additionally screened in wildtype zebrafish (Danio rerio) to provide in vivo toxicity data as well as to observe effects on repetitive behaviors relative to other mAChR PAMs.

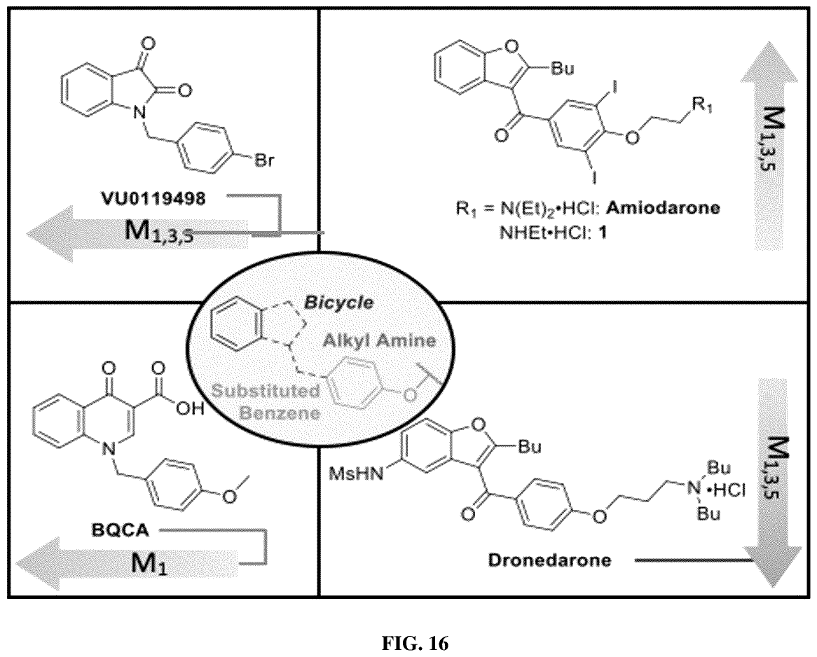

The relative importance of functional groups found in amiodarone was explored through the synthesis of several derivatives. As noted above, amiodarone has an unusual mAChR allosteric profile, detailed below. Although amiodarone has been known to cause pulmonary toxicity with chronic administration, this toxicity appears to be generalized to benzofuran-containing drugs, including amiodarone and dronedarone. Thus, a focused library of hybrid allosteric modulators devoid of benzofurans was designed with a careful consideration of physiochemical properties (i.e., molecular weight, topological surface area, c log P) to enhance CNS penetration. Preliminary evaluations of activity were assessed in vitro by measuring [³H]-arachidonic acid (AA) release in the absence and presence of 0.1 μM and 100 μM ACh in CHO cells expressing M₁ muscarinic receptors. Compounds exhibiting activity in preliminary assays were evaluated further in vitro using full ACh dose response curves. Compounds also were tested in vivo to assess preliminary toxicology and effects on locomotor behaviors in zebrafish (Danio rerio). Based on the structures of BQCA/VU0119498 (potency modulators) and amiodarone/dronedarone (efficacy modulators), a hybrid compound can result in useful additive effects (i.e., potency and efficacy modulation, see FIG. 16).

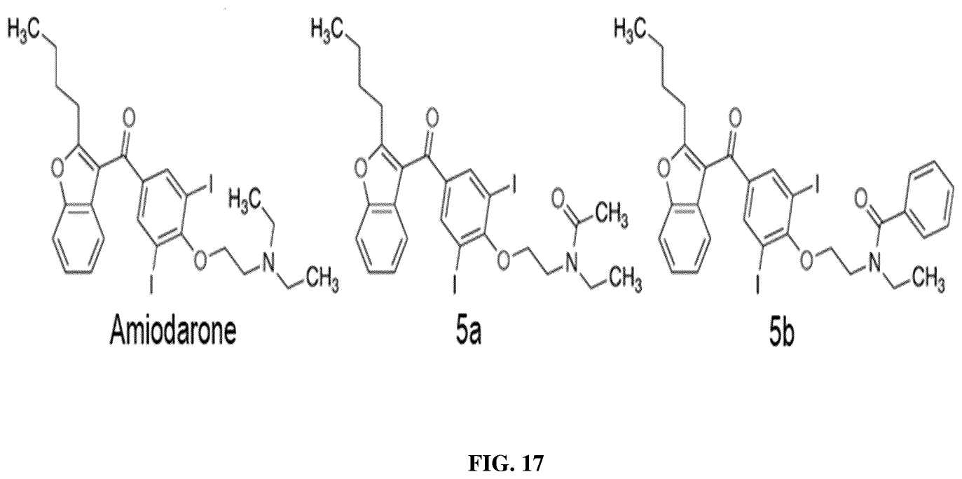

Amiodarone (depicted in FIG. 17) has the property of enhancing M₁ receptor activation by ACh with minimal impact on potency. The acyl derivative 5a (depicted in FIG. 17) was synthesized to replace the tertiary amine found in amiodarone with an acyl amide group. As shown in FIGS. 3A-3B, 5a enhanced the activity of ACh without increasing its potency in CHO cells expressing M₁ muscarinic receptors. The data are consistent with an increase in efficacy cooperativity (β), without appreciable effects on ACh potency (α) or intrinsic efficacy (τB). The corresponding benzyl amide derivative 5b also enhances ACh efficacy at M₁ receptors. Compounds incorporating an isatin ring system in place of the benzofuran group (see FIG. 15) were then synthesized and evaluated. Within this series of compounds, several molecules enhanced ACh efficacy in the [³H]-AA release assay, including 7, 8a, 8j, and 8c.

### Results and Discussion

### Preliminary Screening, Library Design, and Synthesis

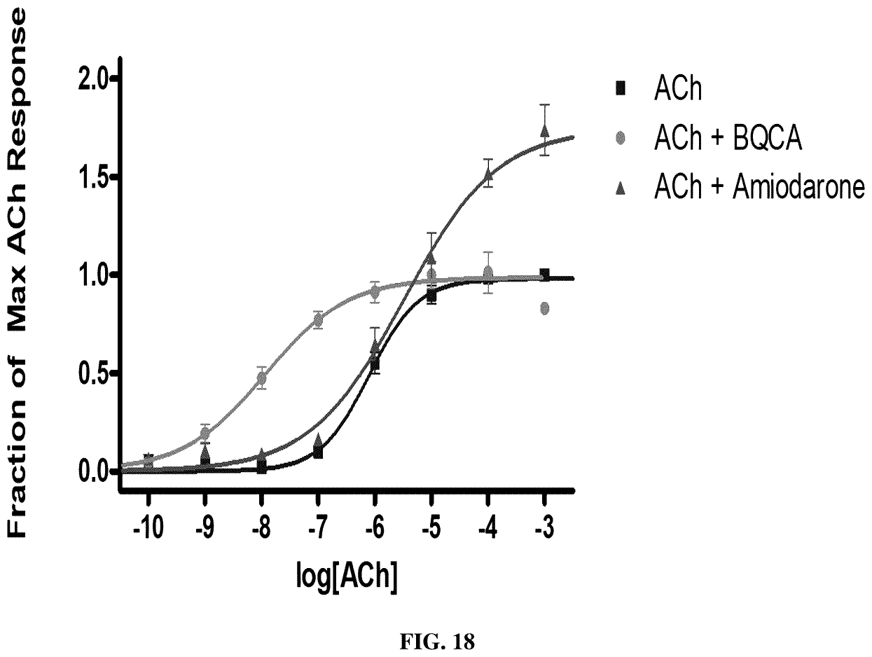

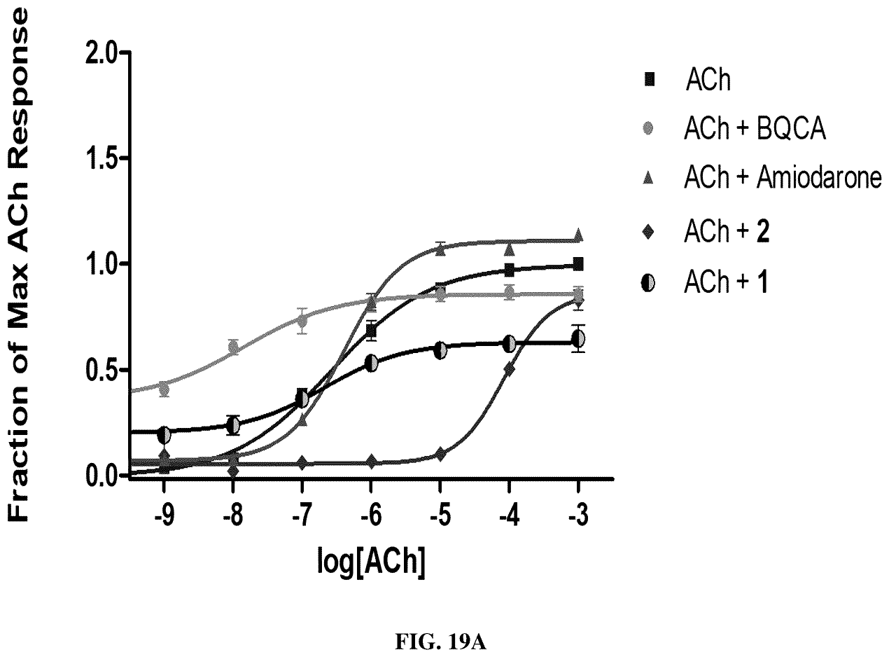

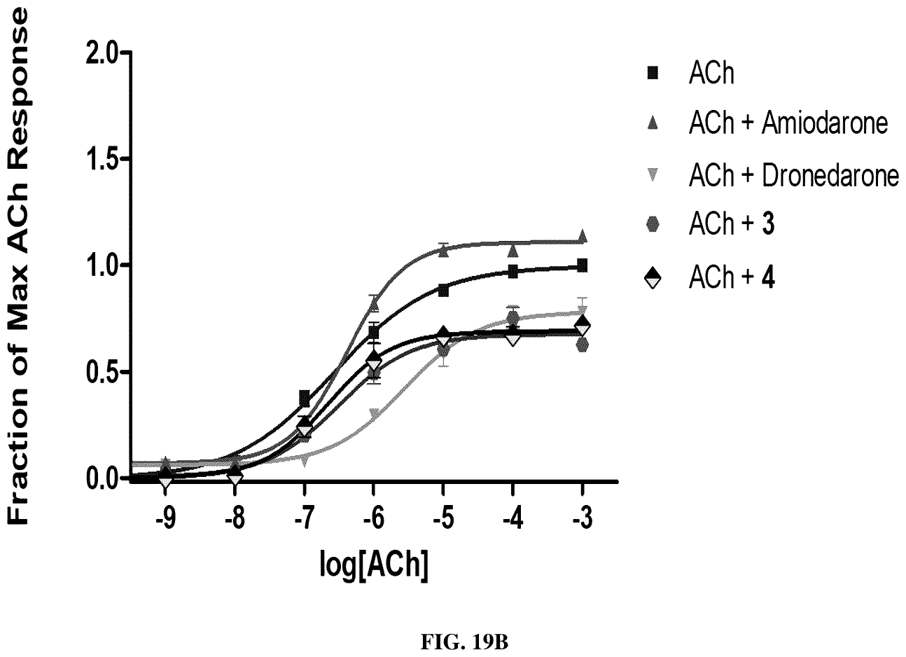

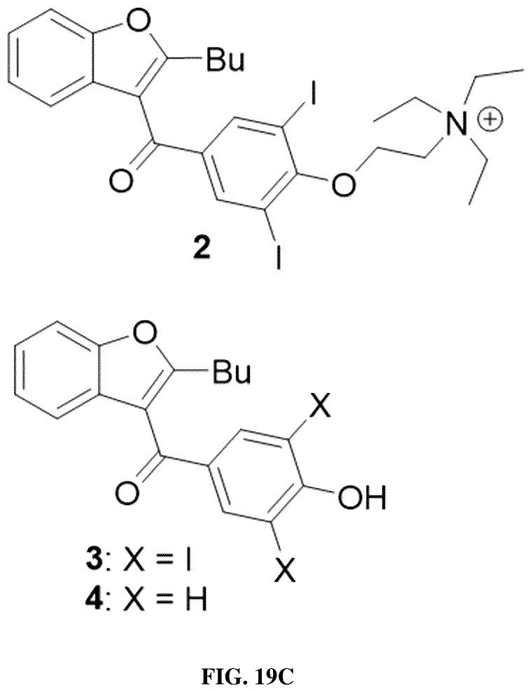

Amiodarone is an antiarrhythmic agent that blocks potassium channels, but it has also been shown to enhance muscarinic receptor activity through an allosteric mechanism. Preliminary studies confirmed that amiodarone is a PAM of ACh at M₁ (FIG. 18A) and M₃ receptors, based on measurements of [³H]-AA release in CHO cells stably transfected with the corresponding receptor subtype. Structurally related compounds were also evaluated, including dronedarone, des-ethyl amiodarone (1). N-ethylamiodarone (2), a related phenol (3), and an analogous phenol lacking the bis-halide substitution (4) (FIGS. 19A-19B). Interestingly, compound 2 was a NAM of mAChR efficacy (FIG. 19A) in a similar fashion as previously reported for dronedarone. Compound 1 was a weaker efficacy PAM relative to amiodarone, and the phenols (3 and 4) were completely inactive (FIG. 19B).

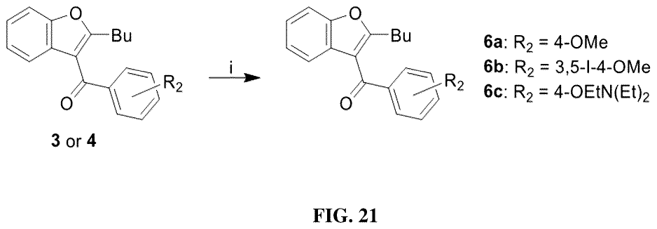

The most compelling aspect of the amiodarone SAR was that modification of the electronics of the amine resulted in both positive allosteric effects (with amiodarone and 1) and negative allosteric effects (with 2 and dronedarone). With this in mind, the amine was modified via either acetylation, 5a, or creation of the benzylamide, 5b. These analogues were synthesized through two synthetic routes. The first route (Scheme 3, FIG. 20) started with the n-dealkylation reaction of amiodarone with 1-chloroethyl chloroformate in refluxing 1,2-dichlorocthane to afford the corresponding carbamate intermediate, followed by a decomposition and reaction with the corresponding anhydride to yield 5a and 5b. Additionally, a second route (Scheme 4, FIG. 21) employed the commercially available 2-butyl-3-benzoyl-benzofurans 3 and 4 which were reacted with cesium carbonate and various alkyl-halides in a Williamson ether synthesis to produce the corresponding ethers (6a-6c) to confirm the SAR observations seen with 3, 4, and amiodarone.

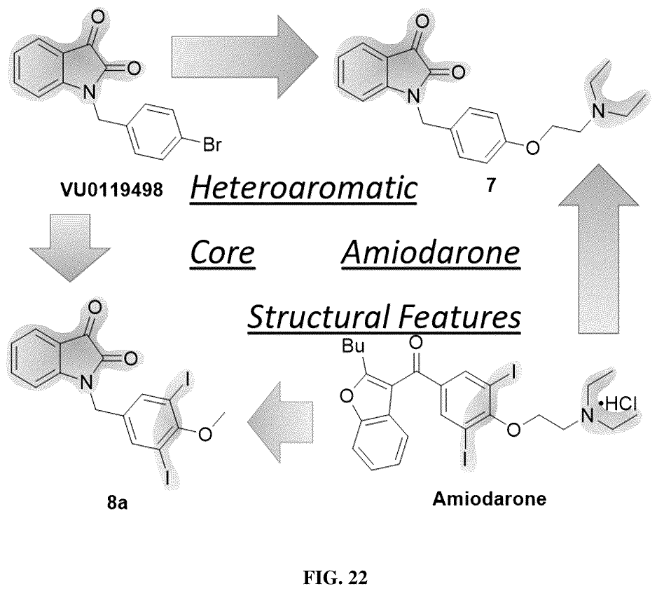

The mAChR allosteric modulators VU0238408 and BQCA contain similar structural components, namely, a bicyclic heterocycle bridged to a para-ether substituted benzene ring. However, despite having similar structural motifs, these compounds exhibit an array of allosteric effects on mAChRs and differ in receptor subtype specificities (or lack thereof). Furthermore, the ether linkage may be extended to feature an alkyl amine, while substitutions may be added to the benzene ring, such as iodides as seen in amiodarone. Such changes, which in addition to influencing allosteric modulation of potency, may also invert modulatory activity from positive to negative (or vice versa). Accordingly, whether the drastic activity cliffs observed in the amiodarone SAR described above applies across other chemical classes of allosteric modulators was evaluated. Amiodarone structural features were combined with isatin, incorporating this heteroaromatic core in the design of potent and selective muscarinic NAMs and PAMs, 7 and 8a, respectively (FIG. 22).

Allosteric modulator hybrids were synthesized via two routes. The first route (Scheme 1, FIG. 4) started with the radical bromination of para-tolyl acetate with N-bromosuccinimide (NBS) to afford the benzyl bromide compound 9. Subsequent alkylation with isatin followed by deacetylation produced compound 10. The final step was a Williamson ether synthesis with the corresponding alkyl chloride to yield 7. The synthesis of 8a and related compounds began with commercially available benzaldehydes (Scheme 2, FIG. 6). Benzaldehydes with unsubstituted phenols were first alkylated in the presence of base and then reduced with sodium borohydride to afford the corresponding benzyl alcohols (11a-11l). The benzyl alcohols were treated with thionyl chloride to yield benzyl chlorides 12a-12l. Commercially available isatin underwent n-alkylation with benzyl halides to produce hybrid compounds 8a-8l.

In vitro evaluation of the library proceeded in two steps: 1) measuring [³H]-AA release in the absence and presence of either 0.1 μM or 100 μM ACh and in the absence or presence of 10 μM of potential allosteric modulator (Table 1); and 2) generation of full ACh dose response curves to characterize active compounds. Surprisingly, 5a and 5b increased mAChR activity by 70% and 80%, respectively, compared to treatment with ACh alone. However, 5a also exhibited significant intrinsic agonist activity. These results, particularly for 5b, which lacked intrinsic activity, were encouraging based on the observation of positive modulation of ACh activity predominantly at higher doses of ACh. Furthermore, these results showed that the electronics of the alkyl-amine are important in modulating mAChR activity. Evaluations of 6a-6c revealed that 6a was inactive, while 6b increased ACh activity, and 6c decreased ACh activity. Compound 7 primarily decreased potency and slightly enhanced the efficacy of ACh activity, whereas 8a resulted in a significant enhancement of ACh activity. Both 7 and 8a lacked intrinsic activity at the 10 μM concentration. The data from the amiodarone derivatives and 8a indicated that the aryl iodides ortho to the phenol ether contributed to the modulation of mAChR activity.

TABLE 1
Stimulation of ³[H]AA release by ACh in the presence and absence
of potential allosteric modulators of M₁ receptors expressed in
CHO cells. Data represent the fraction of stimulation above the
control for each condition. Ratios of the response of ACh with
10 μM modulator to response of ACh alone (0.1 μM or 100
μM) are presented as means ± s.e.m. N = 2-13 for each measurement.

| Compound | Concentration | No ACh | 0.1 μM ACh | 100 μM ACh |
|---|---|---|---|---|
| BQCA | 3 μM | 0.06 ± 0.06 | 2.43 ± 0.19 | 0.99 ± 0.02 |
| | 10 μM | 0.37 ± 0.04 | 2.34 ± 0.23 | 0.86 ± 0.03 |
| Amiodarone | 10 μM | 0.08 ± 0.02 | 0.86 ± 0.08 | 1.13 ± 0.01 |
| | 30 μM | 0.14 ± 0.01 | 1.14 ± 0.05 | 1.37 ± 0.09 |
| CW-2-32 | 10 μM | 0.02 ± 0.03 | 1.07 ± 0.24 | 0.74 ± 0.07 |
| 8j | 10 μM | 0.02 ± 0.02 | 1.06 ± 0.15 | 0.96 ± 0.10 |
| 8a | 10 μM | 0.24 ± 0.04 | 2.64 ± 0.10 | 1.62 ± 0.06 |
| 8b | 10 μM | 0.08 ± 0.01 | 1.81 ± 0.04 | 1.24 ± 0.01 |
| 8c | 10 μM | 0.23 ± 0.00 | 2.15 ± 0.05 | 1.58 ± 0.01 |
| 8d | 10 μM | 0.04 ± 0.01 | 1.31 ± 0.06 | 0.87 ± 0.01 |
| 8e | 10 μM | 0.08 ± 0.01 | 2.13 ± 0.11 | 1.28 ± 0.07 |
| VU0119498 | 10 μM | 0.07 ± 0.00 | 1.22 ± 0.34 | 1.04 ± 0.07 |
| 8f | 10 μM | 0.08 ± 0.02 | 1.49 ± 0.03 | 1.13 ± 0.04 |
| 8g | 10 μM | 0.03 ± 0.01 | 1.33 ± 0.03 | 0.94 ± 0.02 |
| 8h | 10 μM | 0.04 ± 0.01 | 1.17 ± 0.02 | 1.03 ± 0.01 |
| 8i | 10 μM | 0.07 ± 0.01 | 1.39 ± 0.06 | 1.05 ± 0.02 |
| 8k | 10 μM | 0.03 ± 0.00 | 1.37 ± 0.08 | 0.83 ± 0.01 |
| Dronedarone | 10 μM | 0.08 ± 0.01 | 0.25 ± 0.05 | 0.75 ± 0.07 |
| 2 | 10 μM | 0.04 ± 0.02 | 0.17 ± 0.04 | 0.50 ± 0.03 |
| 1 | 10 μM | 0.22 ± 0.01 | 0.75 ± 0.08 | 0.67 ± 0.02 |
| 3 | 10 μM | 0.02 ± 0.00 | 0.42 ± 0.00 | 0.81 ± 0.07 |
| 4 | 10 μM | 0.03 ± 0.02 | 0.49 ± 0.09 | 0.72 ± 0.04 |
| 10 | 10 μM | 0.01 ± 0.01 | 1.60 ± 0.10 | 0.92 ± 0.06 |
| 6c | 10 μM | 0.05 ± 0.04 | 0.46 ± 0.17 | 1.05 ± 0.05 |
| 7 | 10 μM | 0.02 ± 0.01 | 0.08 ± 0.02 | 1.11 ± 0.06 |
| 8l | 10 μM | 0.04 ± 0.00 | 0.19 ± 0.06 | 0.87 ± 0.06 |
| 5a | 10 μM | 0.17 ± 0.04 | 2.22 ± 0.26 | 1.58 ± 0.03 |
| 5b | 10 μM | 0.02 ± 0.01 | 1.34 ± 0.05 | 1.26 ± 0.05 |
| 6b | 10 μM | 0.06 ± 0.01 | 1.6 ± 0.07 | 1.32 ± 0.08 |
| 6a | 10 μM | 0.05 ± 0.01 | 1.18 ± 0.06 | 1.03 ± 0.03 |

A second library was designed featuring a complete series of isatin core-mono/bis o-halogen benzene substitutions (I, Br, Cl, F), 8a-8h, as well as other SAR controls, 8i-8k, to systematically assess the impact of various ring substitutions. An observed increase in ACh activity was favored by bis-aryl-iodide (8a) and bis-aryl-bromide (8c) substitutions, both demonstrating similar amounts of positive allosteric modulation of ACh activity (Table 1). Compounds 8a-8h were rendered in Spartan '20, which was used to calculate various physical and electronic qualities. From these calculated data there is a positive correlation between the area of the molecules and the associated preliminary activity data. This correlation is observed at 10 μM concentration, in the absence of ACh (R²=81.00%), with 0.1 μM ACh (R²=94.74%), and with 100 μM ACh (R²=85.15%). Meanwhile, no correlation was identified with any of the calculated electronic values, indicating that an increase in steric bulk of the halogen substitutions is related to the increase in allosteric modulation of ACh activity.

Perusal of the data from the aniodarone series as well as the hybrid compound libraries, with respect to the observed effects of the aryl-halides as well as the alkyl amines, led to the evaluation of a hybrid compound differing only by the heteroaromatic core. Compound 8l features the isatin heteroaromatic core alongside the bis o-halo(bromo) benzene ring and tertiary alkyl amine. This hybrid compound results only in a decrease in ACh activity, which is more akin to the modulatory effect of 7 and unlike the modulatory effect of 8c. This result further demonstrates the important role of the amine in the evaluated allosteric modulators.

### Rigorous In Vitro Evaluation of Muscarinic Acetylcholine Receptor Modulation

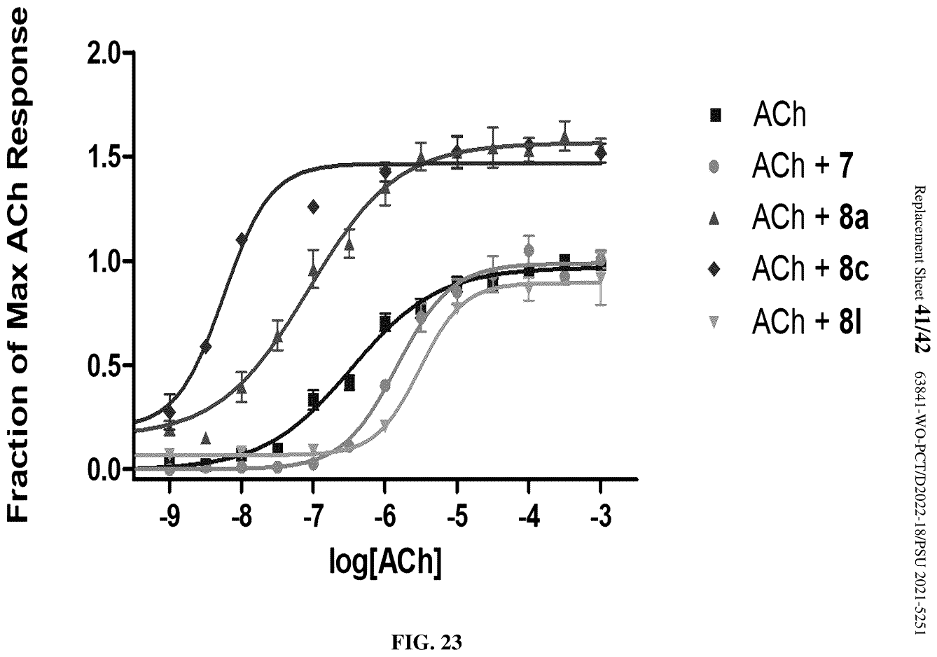

Full ACh dose-response curves were obtained for selected compounds based on the results from the preliminary screen. An interesting range of activities was found as 8a and 8c dramatically enhanced ACh efficacy, while 7 and 8l decreased ACh potency with little impact on ACh efficacy (FIG. 23). These SAR studies revealed clear trends within the isatin heteroaromatic core; alkyl amines predominately lessened ACh potency and, in the absence of alkyl amines, bulkier halides increased ACh efficacy. Within the amiodarone series the trends were clear with one key exception (2). Increases in ACh activity occurs with aryl-halides in conjunction with various amines (tertiary, secondary, acyl) although in conjunction with quaternary amines the ACh activity is lessened. Omission of the phenol ether results in a loss of ACh activity modulation, and omission of the aryl-halides results in the loss ACh efficacy modulation.

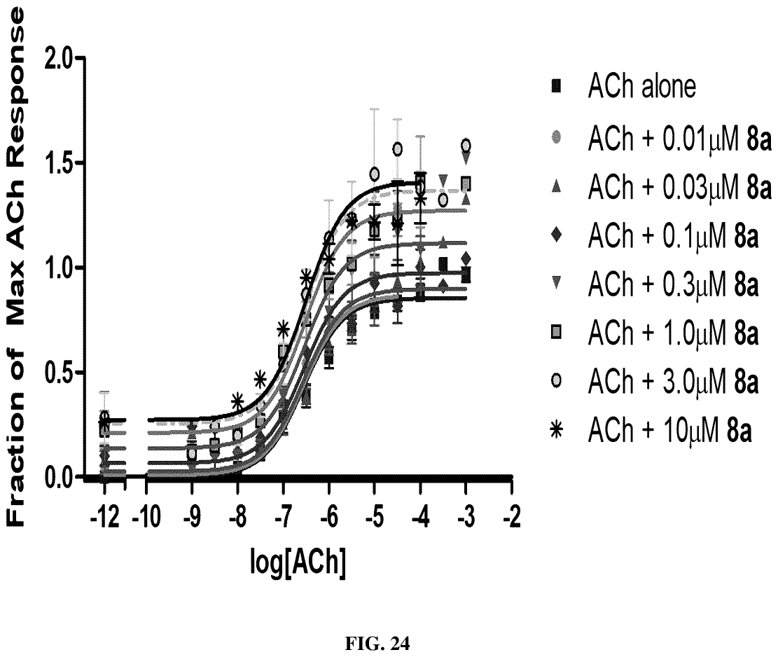

Based on the interesting activities observed at the 10 μM ligand concentration, compounds were evaluated further over a wide range of concentrations to assess the parameters K_{B}, τ_{B}, α, and β by fitting the data to the allosteric operational model. FIG. 24 shows the results for 8a, which clearly potentiates the effects of ACh in a dose-dependent manner. The results from the detailed analyses of 7, 8a, and 8c are summarized in Table 2. As indicated from the initial studies at 10 μM, both 8a and 8c exhibited strong activation cooperativity with ACh, with β values over 3, yet little binding cooperativity with α near unity. 8a exhibited lower intrinsic efficacy (τ_{B}) and higher affinity (K_{B}) than 8c. In contrast, 7 exhibited no activation cooperativity with ACh with a β value near unity, yet displayed negative binding cooperativity with a significantly less than unity. Like 8a, 7 bound with high affinity, yet lacked intrinsic efficacy with τ_{B} near zero.

TABLE 2
Calculated best-fit parameters for three allosteric modulators of ACh
at M₁ muscarinic receptors based on the allosteric operational model.

| Characteristic | Parameter | 7 | 8a | 8c |
|---|---|---|---|---|
| Intrinsic Efficacy-B | τ_{B} | 0.0317 ± 0.0205 | 0.174 ± 0.0289 | 0.328 ± 0.0555 |
| Binding cooperativity, A-B | α | 0.180 ± 0.0855 | 0.689 ± 0.241 | 0.980 ± 0.388 |
| Activation cooperativity, A-B | β | 0.991 ± 0.120 | 3.26 ± 0.431 | 3.89 ± 0.610 |
| Affinity-B | K_{B} | −6.20 ± 0.325 | −6.26 ± 0.152 | −5.53 ± 0.148 |
| Goodness of global fit | R² | 0.923 | 0.860 | 0.922 |

The mean (±s.e.m.) intrinsic efficacy of ACh (τ_{A}) was 0.822 ± 0.092, while the mean (±s.e.m.) affinity of ACh (logK_{A}) was −6.23 ± 0.0895, and the efficacy of the system (Eₘ) was set to 2.0 across the three evaluations.

### In Vivo Toxicology and Effects on Locomotor Behavior

All compounds were tested for effects on locomotor activity and repetitive behaviors in wild-type zebrafish (Danio rerio). BQCA, a well-known, selective PAM of M₁ muscarinic receptors, was used as a benchmark for these tests. BQCA decreased overall locomotor activity during the 30-minute spontaneous swimming period in AB zebrafish (FIGS. 10A, 10C). These data are consistent with a role for M₁ muscarinic receptors in regulating locomotor activity. BQCA treatment also increased angular velocity (FIG. 10D) and variance of turn angle in AB zebrafish during the 30-minute spontaneous swimming period, indicating a decrease in repetitive behaviors. The data for the entire series of compounds are shown in Table 3. Of note, the PAM of ACh efficacy 8a also reduced locomotor activity (FIGS. 10B, 10E) and increased angular velocity (FIG. 10F) and variance of turn angle in AB zebrafish during the spontaneous swimming period, again reflecting a decrease in repetitive behaviors. Compound 8a was more potent than BQCA with significant effects at 1 μM, which is consistent with its affinity for M₁ muscarinic receptors (K_{B} of 589 nM). Additional studies were conducted using lower concentrations of 8a to establish EC₅₀ values for decreasing locomotor activity (5.6 μM) and repetitive behaviors (2.6 μM for increasing angular velocity). In addition, compound 8a did not produce any signs of toxicity at concentrations up to 30 μM, the highest concentration tested.

TABLE 3
Effects of potential allosteric modulators of M₁ receptors on locomotor activity
in zebrafish. Maximum tolerated concentration was determined after 24-hour exposure.
Data represent the percentage change in locomotor behaviors relative to controls
and are presented as means ± s.e.m. N = 12 for each measurement.

| | Maximum | | | |
|---|---|---|---|---|
| | Tolerated | Total Distance | | Variance of |
| | Concentration | Moved | Angular Velocity | Turn Angle |
| Compound | (μM) | (% of control) | (% of control) | (% of control) |
| BQCA | 10 | 72.0 ± 10.0* | 141.8 ± 14.9* | 119.6 ± 7.0* |
| Amiodarone | 10 | 85.7 ± 10.2 | 83.2 ± 12.0 | 102.2 ± 10.4 |
| CW-2-32 | 30 | 34.6 ± 4.0*** | 180.1 ± 20.3*** | 146.6 ± 12.4*** |
| 8a^{b} | 30 | 45.8 ± 6.4*** | 197.1 ± 19.8*** | 161.1 ± 13.4*** |
| 8b | 10 | 52.7 ± 6.6*** | 175.3 ± 19.0** | 143.0 ± 13.5** |
| 8c^{b} | 30 | 26.9 ± 3.7*** | 201.6 ± 20.2*** | 150.8 ± 12.0*** |
| 8d | 100 | 38.4 ± 4.5*** | 201.4 ± 27.5*** | 170.2 ± 15.1*** |
| 8e | 100 | 29.1 ± 5.3*** | 222.1 ± 25.0*** | 158.8 ± 14.3*** |
| 8f | 10 | 37.4 ± 6.4*** | 157.1 ± 24.5* | 127.9 ± 16.2 |
| 8g | 30 | 65.7 ± 11.7*** | 144.5 ± 18.6** | 126.5 ± 10.4* |
| 8h | 30 | 52.7 ± 6.6*** | 201.4 ± 27.5** | 148.0 ± 12.4*** |
| 8i | 10 | 11.6 ± 1.2*** | 412.1 ± 28.7*** | 146.1 ± 16.1** |
| 8k | 30 | 66.2 ± 8.4** | 154.2 ± 18.0** | 126.3 ± 10.8* |
| Dronedarone | 10 | 28.3 ± 5.3*** | 172.8 ± 25.7*** | 141.6 ± 12.5*** |
| 2^{a} | 1 | 95.8 ± 11.0 | 128.9 ± 18.5 | 97.6 ± 10.3 |
| 1 | 3 | 114.1 ± 8.4 | 84.5 ± 5.5 | 84.4 ± 6.3 |
| 3^{a} | 3 | 145.4 ± 21.7 | 101.8 ± 19.2 | 93.4 ± 9.4 |
| 4^{a} | 0.3 | 56.7 ± 9.3 | 105.7 ± 14.8 | 115.8 ± 12.4 |
| 10 | 10 | 72.4 ± 8.6* | 131.2 ± 13.4* | 135.3 ± 13.5* |
| 6c^{a} | 3 | 59.8 ± 8.8 | 129.9 ± 20.1 | 136.4 ± 11.8 |
| 7 | 10 | 72.5 ± 11.8 | 122.4 ± 6.9 | 93.9 ± 10.6 |
| 8l | 10 | 33.5 ± 4.4** | 184.7 ± 17.0*** | 166.1 ± 12.5*** |
| 5a | 10 | 108.0 ± 12.9 | 104.0 ± 10.7 | 102.5 ± 8.1 |
| 5b | 10 | 87.7 ± 13.5 | 80.4 ± 11.5 | 77.8 ± 9.3 |
| 6b | 30 | 75.3 ± 8.5* | 132.3 ± 16.9 | 133.7 ± 14.0* |
| 6a | 3 | 115.4 ± 11.7 | 95.9 ± 13.3 | 115.8 ± 13.0 |

n.d. = not determined
n.a. = not active
^{a}tested at 1 μM due to toxicity at 10 μM
^{b}denotes insolubility at 100 μM, i.e., 30 μM was the highest dose tested
*p < 0.05,
**p < 0.01,
***p < 0.001 compared to control

### CONCLUSION

In summary, evaluation of hybrid mAChR allosteric ligands elucidated key structural motifs that result in distinct allosteric profiles. Principally the bis-ortho iodo/bromo phenol ether exhibited a drastic positive activation cooperativity, while the phenoxy ethylamine produced negative binding cooperativity. Combination of these structural motifs did not result in additive effects; rather, surprisingly, the negative allosteric modulatory effects on potency predominated within the isatin heteroaromatic derivatives (8l) while the positive allosteric modulatory effects predominated within the benzofuran series (amiodarone). Most notably, the two drug-like compounds 8a and 8c displayed positive activation cooperativity with ACh at the M₁ mAChR, demonstrated low in vivo toxicity, and diminished repetitive behaviors in the zebrafish model. These compounds are useful as mAChR allosteric therapeutic ligands for disease states associated with deficits/alterations in cholinergic signaling.

### Additional Compounds

Four additional compounds were synthesized and characterized for pharmacological activity at M1 muscarinic receptors. The compounds were named GK-1-44, GK-1-48, GK-1-47, and GK-1-50, and are shown below:

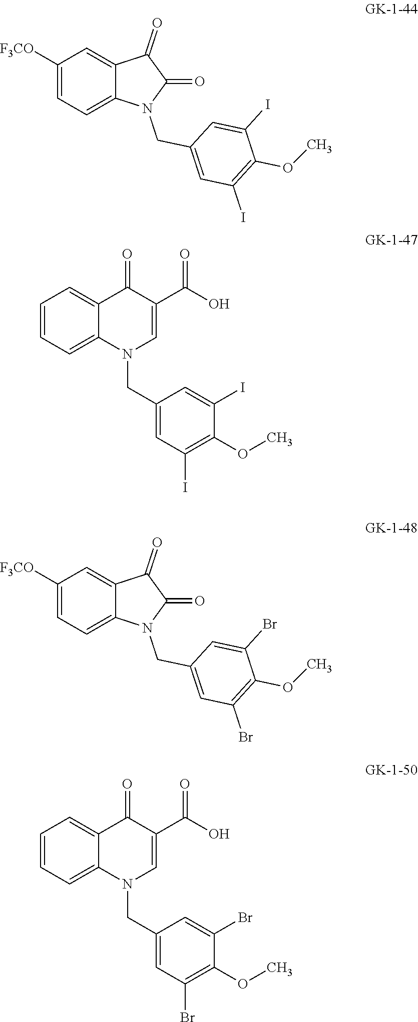 [SMILES: COc1c(I)cc(CN2C(=O)C(=O)c3cc(OC(F)(F)F)ccc32)cc1I ; COc1c(I)cc(Cn2cc(C(=O)O)c(=O)c3ccccc32)cc1I ; COc1c(Br)cc(CN2C(=O)C(=O)c3cc(OC(F)(F)F)ccc32)cc1Br ; COc1c(Br)cc(Cn2cc(C(=O)O)c(=O)c3ccccc32)cc1Br]

The activity of these four compounds in stimulating the release of [³H]-arachidonic acid from CHO cells expressing M₁ muscarinic receptors is summarized in Table 4 below. The 5-trifluoromethoxy-isatin derivatives (GK-1-44 and GK-1-48) were more effective in enhancing the effects of 100 μM acetylcholine (ACh) than the corresponding benzoquinolone carboxylic acid derivatives (GK-1-47 and GK-1-50).

TABLE 4
Stimulation of ³[H]AA release by ACh in the presence and absence
of potential allosteric modulators of M₁ receptors expressed
in CHO cells. Data represent the fraction of stimulation above
the control for each condition. Ratios of the response of ACh
with 10 μM modulator to response of ACh alone (0.1 μM or
100 μM) are presented as means ± s.e.m. N = 4-5 for each measurement.

| Compound | Concentration | No ACh | 0.1 μM ACh | 100 μM ACh |
|---|---|---|---|---|
| GK-1-44 | 10 μM | 0.37 ± 0.04 | 2.47 ± 0.22 | 1.67 ± 0.11 |
| GK-1-47 | 10 μM | 0.24 ± 0.01 | 2.46 ± 0.13 | 1.33 ± 0.04 |
| GK-1-48 | 10 μM | 0.15 ± 0.01 | 3.04 ± 0.47 | 1.66 ± 0.07 |
| GK-1-50 | 10 μM | 0.47 ± 0.10 | 2.77 ± 0.49 | 1.11 ± 0.12 |

Three of the compounds were evaluated for their effects on zebrafish locomotor activity and turning behavior. The data are summarized in Table 5. Of the three compounds, GK-1-48 produced a significant increase in angular velocity, reflecting a decrease in repetitive behaviors.

TABLE 5
Effects of allosteric modulators of M₁ receptors on locomotor activity in zebrafish
(the data shown are for the 10 μM concentration). Maximum tolerated concentration
was determined after 24-hour exposure in separate experiments. Data represent
the percentage change in locomotor behaviors relative to controls and are
presented as means ± s.e.m. N = 12 for each measurement.

| | Maximum | | | |
|---|---|---|---|---|
| | Tolerated | Total Distance | | Variance of |
| | Concentration | Moved | Angular Velocity | Turn Angle |
| Compound | (μM) | (% of control) | (% of control) | (% of control) |
| GK-1-44 | 10 | 78.5 ± 11.1 | 114.1 ± 19.2 | 105.2 ± 10.6 |
| GK-1-47 | — | — | — | — |
| GK-1-48 | 3 | 80.1 ± 11.7 | 133.9 ± 16.5* | 109.4 ± 11.9 |
| GK-1-50 | 10 | 87.9 ± 10.7 | 124.1 ± 21.8 | 107.4 ± 11.2 |

### Materials and Methods

### Cell Culture

CHO cells expressing human M₁ muscarinic receptors were cultured at 37° C. in a 5% CO₂ atmosphere in F12 medium supplemented with 5% fetal bovine serum, 100 units/mL penicillin, and 100 μg/mL streptomycin.

### Arachidonic Acid Release

Measurement of [³H]AA release was performed. Stably transfected CHO-hM₁ or CHO-hM₃ cells were seeded on 24-well plates (Greiner Bio-One GmbH, Frickenhausen, Germany) at a density of 5.0×10⁴ cells/well in 0.5 ml of F-12 medium. Cells were incubated until they attached (approximately 3 h), followed by the addition of [³H]AA to a final concentration of 0.05 μCi of [³H]AA per well. The cells were then grown for 16 to 20 h before the assay was performed. [³H]AA release was measured in Eagle's basal medium with 20 mM HEPES and 2 mg/ml fatty acid-free bovine serum albumin (EM-BSA). Cells were rinsed twice with EM-BSA, followed by the addition of EM-BSA media containing experimental agents and incubated for 1 h at 37° C. The assay was terminated by aspiration of the media, and the amount of [³H]AA released was determined by liquid scintillation counting (Beckman-Coulter LS6500).

For some assays, pretreatment with phenoxybenzamine (POB) was employed, as indicated, to reduce the number of available receptors per cell. Stock POB was made up at 10 mM in ethanol and stored at −20° C. Each well was pretreated with 0.5 ml of the appropriate concentration in PBS with 1 mM CaCl₂ and 1 mM MgCl₂ (PBS++) for 30 min at 37° C., followed by 5 washes with PBS++. After pretreatment, the normal protocol was followed as above.

### In Vitro Pharmacological Data Analysis

Tabulated [³H]AA release data are represented as a fraction of maximal release in the presence of 100 μM ACh or as the fraction of release elicited by the appropriate concentration of ACh alone, as indicated dose-response curves were normalized to the maximal response of ACh without allosteric ligand. Some response curves were analyzed using the empirical four-parameter equation:

$$Y = B o t t o m + ( T op - Bottom ) / ( 1 + {10}^{n ( logC 50 - X )} )$$

where X is the log of the concentration of the ligand used, Y is the amount of response, C₅₀ is the concentration of the ligand that produces 50% of the maximal effect, Top and Bottom are the top and bottom plateaus of the curve, respectively, and n is related to the Hill slope for the curve. In some cases, families of response curves were analyzed according to the allosteric operational model:

$$Y = {E}_{M} [ {τ}_{A} A ( {K}_{B} + α β B ) + {τ}_{B} B {K}_{A} ) ] / [ {τ}_{A} A ( {K}_{B} + α β B ) + {τ}_{B} B {K}_{A} + A {K}_{B} + {K}_{A} {K}_{B} + {K}_{A} B + α A B ]$$

where E_{M} is the maximal response; A is the concentration of ACh; B is the concentration of the allosteric ligand; τ_{A} and τ_{B} represent the intrinsic efficacies of A and B, respectively; K_{A} and K_{B} are the equilibrium dissociation constants of A and B, respectively; and α and β represent the cooperativities between A and B in terms of binding and activation, respectively. Curve-fitting was carried out with GraphPad Prism, version 5.0 or 6.0 (San Diego CA).

Disintegration per minute (DPM) values for a single compound were obtained in triplicate, and each compound was evaluated on three separate 24-well plates. The mean values for each condition from each of the three 24-well plates were used for the statistical analysis. The baseline mean was subtracted from each value, then normalized to the mean response produced by 100 μM ACh (set at 1.0). Statistical analyses were completed on the normalized values via one-way repeated measures ANOVA with Tukey's post hoc test. Statistically significant differences are noted as follows: *, p<0.05; **, p<0.01; ***, p<0.001.

### Animals and Husbandry

Larval zebrafish were produced in the University of Toledo zebrafish core facility and housed in an incubator maintained at 28° C. on a 14:10 h cycle. Fish were acclimated to the testing room conditions the evening prior to testing. All testing took place during the 14 h light cycle after the lights had been on for at least 2 h. All experiments and husbandry practices were approved by the Institutional Animal Care and Use Committee (IACUC #400091).

### Maximum Tolerated Concentration (MTC) Determination

Determination of maximum tolerated concentration was completed by placing 5 days post fertilization (dpf) wild-type zebrafish larvae in a 24 well plate at 6 fish per well, and one compound concentration per well. Seven concentrations were tested ranging from 0.1 μM to 1 mM at half log intervals, solubility permitting. Death was determined by the absence of heartbeat. Additional endpoints were used to assess toxicity at non-lethal concentrations such as startle capacity (poke stimulus), abnormalities in swimming behavior (loss of dorsoventral balance), and gross morphological deformities (bent body). The MTC was defined as the highest concentration that did not cause death and where not more than 2 out of 12 larvae exhibit any sign of locomotor impairment, including no touch response after 24 hours. The MTC determination was used to select three doses of each compound (low, medium, high dose) to be used in behavioral assays.

### General Locomotor Assay

Five days post fertilization (dpf) zebrafish larvae were exposed to a range of nonlethal concentrations of the compounds in a 24-well plate with appropriate controls. The movement of each larval zebrafish was monitored with the Noldus behavior recording system (Noldus Information Technology, Leesburg VA) and quantified using EthoVision® XT 15 software (Noldus Information Technology, Leesburg VA). The plate was immediately placed in the incubator for a 30-minute exposure period. After 30 minutes, the plate was transferred to the Noldus system and swimming behavior was recorded in 100% light for 30 minutes to measure spontaneous swimming behavior. The light was switched off for 10 minutes (dark period) and then switched on for 10 minutes (light period), which was then repeated for two additional cycles. In total, the larvae were exposed to three cycles of alternating 10-minute dark and light periods (100% darkness-to-100% light). Each drug challenge was conducted on three separate plates with n=6 larvae/dose/plate.

### Zebrafish Behavior Statistical Analysis

All data for exposure and the behavioral end points were analyzed by one-way ANOVA (factor: drug conc) followed by a Dunnett's multiple comparisons test for post hoc significance between drug concentration and control group. Statistically significant differences are noted as follows: *, p<0.05; **, p<0.01; ***, p<0.001.

### General Methods

Reagents and solvents were purchased from common commercial suppliers Fisher (Durham, NC), or Sigma-Aldrich (St. Louis, MO) and used as received. All reactions were carried out under atmospheric conditions at room temperature unless otherwise indicated. Reactions were monitored by thin-layer chromatography (TLC, LuxPlate silica gel 60 F₂₅4 plates) and revealed by UV light (254 nm). Column chromatography was performed using Teledyne Combiflash R_{f} with RediSepR_{f} Gold columns. HPLC analysis was performed using a Shimadzu Prominence HPLC (LCD-20AD) with temperature controlled autosampler (SIL-20AC), refractive index (RID-20A), and PDA (SPD-M20A) detectors. Separations utilized a Phenomenex Kinetix® core column (2.6 μm, C18, 100 Å, 100×4.6 mm column). HPLC conditions: mobile phase A=H₂O (0.1% formic acid [FA]) and mobile phase B=acetonitrile (0.1% FA); 1.0 mL/min at 30% B for 1 min followed by gradient increase to 95% B over 6 min followed by 1 min at 95% B and re-equilibration at 30% B for 4 min resulting in a total run time of 12 min. Purity of tested compounds was found to be >95% pure at two wavelengths, 254 nm and 280 nm, unless otherwise indicated, or by elemental analysis performed by AtlanticMicrolabs (Norcross, GA). NMR (H, ¹³C) was taken using a Bruker Avance 600 MHz spectrometer (cryoprobe). High resolution mass spectra (HRMS) were recorded using Waters Synapt high-definition mass spectrometer (HDMS) equipped with nano-ESI source positive mode. Compounds 5a and 5b have cis and trans isomeric states that interconvert, each with distinctive NMR spectra.

### Synthetic Chemistry

### (2-butylbenzofuran-3-yl)(4-(2-(ethylamino)ethoxy)-3,5-diiodophenyl)methanone hydrochloride (1)

Amiodarone HCl (500.0 mg, 733.4 μmol) was stirred in a biphasic solution of EA (30 mL) and saturated NaHCO₃ (aq, 20 mL) for 1 hour. The EA layer was washed with brine, dried over anhydrous Na₂SO₄, and concentrated in vacuo to afford a yellow oil, amiodarone free base. The residue was taken up in DCE (10 mL) and cooled down to 0° C. with stirring. To this solution 1-chloroethyl carbonochloridate (395.6 μL, 3.7 mmol) was added and allowed to stir for 30 minutes. The reaction mixture was then heated to reflux for 1 hour. After cooling to room temperature, the reaction mixture was concentrated in vacuo, dissolved in anhydrous MeOH (10 mL), and refluxed with stirring for 1 hour. After cooling to room temperature, the reaction mixture was concentrated in vacuo and purified by flash chromatography (SiO₂, EA/MeOH; 0-30%) to afford 1 as white crystal (75.6 mg, 15.8%). ¹H NMR (DMSO-d₆, 600 MHz): δ 8.99 (2H, b); 8.20 (2H, s); 7.68-7.67 (1H, d, J=8.22 Hz); 7.51-7.50 (1H, d, J=7.8 Hz); 7.39-7.37 (1H, m); 7.32-7.30 (1H, m); 4.28-4.27 (2H, m); 3.49-3.47 (2H, t, J=4.38 Hz); 3.16-3.12 (2H, q, J=7.14 Hz); 2.73-2.70 (2H, t, J=7.62 Hz); 1.72-1.67 (2H, quint, J=7.56 Hz); 1.30-1.23 (5H, m); 0.86-0.84 (3H, t, J=7.32 Hz). ¹³C NMR (DMSO-d₆, 600 MHz): δ187.9, 166.1, 160.4, 153.5, 140.2, 139.2, 126.6, 125.4, 124.4, 121.3, 116.0, 111.7, 92.7, 68.6, 46.6, 42.8, 40.5, 29.8, 28.0, 22.5, 13.9, 11.5. Measured purity by elemental analysis: calcd for (C: 42.27, H: 3.98, N: 2.14, Cl: 5.43, I: 38.84); found (C: 42.06, H: 3.86, N: 2.11, Cl: 5.43, I: 38.71).

### N-(2-(4-(2-butylbenzofuran-3-carbonyl)-2,6-diiodophenoxy)ethyl)-N-ethylacetamide (5a)

1 (250.0 mg, 382.4 μmol) was dissolved in DCM (5 mL) and cooled to 0° C. Et₃N (117.3 μL, 841.3 mol) was added to the reaction mixture and allowed to stir for 10 minutes, then Ac₂O (54.2 μL, 573.6 μmol) was added and allowed to raise to room temperature over 16 hours. The reaction mixture was concentrated in vacuo and purified by flash chromatography (SiO₂, Hex/EA; 0-30%) to afford 5a as yellow oil (102.7 mg, 40.7%). 1H NMR (CDCl₃ DMSO-d₆, 600 MHz): δ8.20-8.19 (2H, m); 7.48-7.46 (1H, m); 7.39-7.38 (1H, d, J=7.84z); 7.31-7.27 (1H, m); 7.24-7.21 (1H, m); 4.22-4.20 (1.3H, t, J=5.10 Hz); 4.15-4.14 (0.7H, t, J=5.83 Hz); 3.83-3.80 (2H, m); 3.61-3.53 (2H, m); 2.86-2.82 (2H, q, J=6.98 Hz); 2.24 (1H, s); 2.15 (2H, s); 1.78-1.73 (2H, m); 1.38-1.31 (2H, m); 1.28-1.25 (2H, t, J=6.98 Hz); 1.19-1.17 (1H, t, J=7.76 Hz); 0.91-0.89 (3H, m). ¹³C NMR (CDCl₃ DMSO-d₆, 600 MHz): δ187.7, 187.6, 170.6, 166.3, 166.2, 160.6, 160.2, 153.6, 140.7, 138.7, 138.4, 126.3, 124.7, 123.8, 121.0, 115.8, 115.7, 111.1, 90.8, 90.7, 71.8, 70.5, 47.5, 45.6, 45.5, 41.2, 30.0, 28.2, 22.5, 22.0, 21.4, 14.1, 13.7, 12.8. Measured purity at 254 nm: 97.32%; 280 nm: 99.9%. HRMS: calcd for (C₂₅H₂₇I₂NO₄): 659.0029, found 681.9944 (M+Na⁺).

### N-(2-(4-(2-butylbenzofuran-3-carbonyl)-2,6-diiodophenoxy)ethyl)-N-ethylbenzamide (5b)

1 (958.9 mg, 1.47 mmol) was dissolved in DCM (10 mL) and cooled to 0° C. Et₃N (408.9 μL, 2.93 mmol) was added to the reaction mixture and allowed to stir for 10 minutes, then Bz₂O (165.9 mg. 733.4 μmol) was added and allowed to raise to room temperature over 16 hours. The reaction mixture was concentrated in vacuo and purified by flash chromatography (SiO₂, Hex/EA; 0-30%) to afford 5b as yellow oil (145.2 mg, 13.7%). ¹H NMR (CDCl₃ DMSO-d₆, 600 MHz): δ8.22-8.18 (2H, m); 7.50-7.46 (3H, m); 7.42-7.40 (4H, m); 7.32-7.30 (1H, m); 7.24-7.23 (1H, m); 4.38 (2H, s); 4.03-4.02 (2H, m); 3.80 (1H, s); 3.59-3.56 (1H, m); 2.87-2.84 (2H, t, J=7.42 Hz); 1.80-1.75 (2H, pent, J=7.49 Hz); 1.40-1.33 (3H, m); 1.25-1.23 (3H, m); 0.93-0.91 (3H, t, J=7.49 Hz). ¹³C NMR (CDCl₃ DMSO-d₆, 600 MHz): δ187.6, 187.5, 171.9, 166.2, 166.0, 161.4, 160.6, 153.6, 140.7, 138.2, 136.7, 129.4, 128.4, 126.8, 126.5, 126.4, 126.3, 124.7, 123.8, 121.1, 115.8, 111.1, 91.0, 71.6, 60.3, 52.0, 51.9, 47.7, 45.9, 45.1, 30.0, 28.2, 28.0, 22.6, 22.5, 13.7, 12.0. Measured purity at 254 nm: 98.34%; 280 nm: 97.25%. HRMS: calcd for (C₃₀H₂₉I₂NO₄): 721.0186, found 744.0033 (M+Na⁺).

### (2-butylbenzofuran-3-yl)(4-methoxyphenyl)methanone (6a)

4 (1.00 g, 3.4 mmol) was dissolved in anhydrous DMF (10 mL), added to a RBF with Cs₂CO₃ (2.21 g, 6.8 mmol), and heated to 60° C. To the reaction mixture methyl iodide (846.0 μL, 13.6 mmol) was added and allowed to stir for 1 hour. After cooling to room temperature, the reaction mixture was quenched with DI (20 mL) and extracted with EA (2×20 mL). The combined organic extracts were washed with DI (2×20 mL), brine (20 mL), dried over anhydrous Na₂SO₄, and concentrated in vacuo to afford a yellow oil. The residue was purified by flash chromatography (SiO₂, Hex/EA; 0-30%) to afford 6a as clear oil (621.8 mg, 59.2%). ¹H NMR (CDCl₃, 600 MHz): δ 7.86-7.84 (2H, m); 7.49-7.47 (1H, d, J=8.22 Hz); 7.37-7.35 (1H, d, J=7.82 Hz); 7.29-7.26 (1H, m); 7.20-7.17 (1H, m); 6.98-6.95 (2H, m); 3.90 (3H, s); 2.93-2.90 (2H, J=7.49 Hz); 1.78-1.73 (2H, quint, J=8.02 Hz); 1.39-1.33 (2H, sext, J=7.23 Hz); 0.91-0.88 (3H, t, J=8.02 Hz). ¹³C NMR (CDCl₃, 600 MHz): δ 190.5, 164.7, 163.4, 153.5, 131.8, 131.7, 127.1, 124.1, 123.3, 121.2, 116.7, 113.6, 110.9, 55.5, 30.1, 27.8, 22.3, 13.7. Measured purity at 254 nm: 95.6%; 280 nm: 95.9%. HRMS: calcd for (C₂₀H₂₀O₃): 308.1412, found 309.1501 (M+H⁺).

### (2-butylbenzofuran-3-yl)(3,5-diiodo-4-methoxyphenyl)methanone (6b)

3 (1.21 g, 2.2 mmol) was dissolved in anhydrous DMF (10 mL), added to a RBF with K₂CO₃ (612.4 mg, 4.4 mmol), and heated to 60° C. To the reaction mixture methyl iodide (551.7 μL, 8.9 mmol) was added and allowed to stir for 1 hour. After cooling to room temperature, the reaction mixture was quenched with DI (20 mL) and extracted with EA (2×20 mL). The combined organic extracts were washed with DI (2×20 mL), brine (20 mL), dried over anhydrous Na₂SO₄, and concentrated in vacuo to afford a yellow oil. The residue was purified by flash chromatography (SiO₂, Hex/EA; 0-30%) to afford 6b as yellow oil (495.1 mg, 39.9%). ¹H NMR (CDCl₃, 600 MHz): δ 8.22 (2H, s); 7.50-7.49 (1H, d, J=8.21 Hz); 7.44-7.43 (1H, d, J=7.30 Hz); 7.33-7.30 (1H, m); 7.27-7.24 (2H, m); 3.95 (3H, s); 2.87-2.85 (2H, t, J=7.69 Hz); 1.81-1.75 (2H, m); 1.40-1.34 (2H, sext, J=7.41 Hz); 0.94-0.91 (3H, t, J=7.41 Hz). ¹³C NMR (CDCl₃, 600 MHz): δ187.8, 166.2, 162.3, 153.6, 140.7, 138.4, 126.4, 124.7, 123.8, 121.0, 115.8, 111.1, 90.5, 60.8, 30.0, 28.2, 22.5, 13.7. Measured purity at 254 nm: 95.7%; 280 nm: 98.9%. HRMS: calcd for (C₂₀H₁₈I₂O₃): 559.9345, found 560.9465 (M+H⁺).

### (2-butylbenzofuran-3-yl)(4-(2-(diethylamino)ethoxy)phenyl)methanone (6c)

4 (1.00 g, 3.4 mmol) was dissolved in anhydrous DMF (10 mL), added to a RBF with Cs₂CO₃ (4.43 g, 13.6 mmol), and heated to 60° C. To the reaction mixture 2-chloro-N,N-diethylethan-1-anine hydrochloride (584.7 mg, 3.4 mmol) was added and allowed to stir for 1 hour. After cooling to room temperature, the reaction mixture was quenched with DI (20 mL) and extracted with EA (2×20 mL). The combined organic extracts were washed with DI (2×20 mL), brine (20 mL), dried over anhydrous Na₂SO₄, and concentrated in vacuo to afford a yellow oil. The residue was purified by flash chromatography (SiO₂, Hex/EA; 0-30%) to afford 6c as yellow oil (671.5 mg, 50.1%). 1H NMR (CDCl₃, 600 MHz): δ 7.83-7.81 (2H, d, J=8.72 Hz); 7.47-7.46 (1H, d, J=8.20 Hz); 7.35-7.34 (1H, d, J=7.65 Hz); 7.27-7.25 (1H, m); 7.19-7.16 (1H, m); 6.96-6.94 (2H, d, J=8.74 Hz); 4.13-4.11 (2H, t, J=6.19 Hz); 2.91-2.89 (4H, t, J=6.73 Hz); 2.67-2.63 (4H, q, J=7.40 Hz); 1.77-1.72 (2H, m); 1.37-1.32 (2H, m); 1.09-1.07 (6H, t, J=7.73 Hz); 0.90-0.87 (3H, t, J=7.73 Hz). ¹³C NMR (CDCl₃, 600 MHz): δ 190.5, 164.6, 162.8, 153.5, 131.8, 131.6, 127.2, 124.1, 123.3, 121.2, 116.7, 114.2, 110.9, 66.9, 51.5, 47.9, 30.1, 27.8, 22.3, 13.7, 11.8. Measured purity at 254 nm: 97.2%; 280 nm: 98.9%. HRMS: calcd for (C₂₅H₃₁NO₃): 393.2304, found 416.2211 (M+Na⁺).

### 4-(bromomethyl)phenyl acetate (9)

p-Tolyl acetate (11.28 g, 75.11 mmol) was dissolved in CCl₄ (100 mL), and added to a RBF containing NBS (14.71 g, 82.6 mmol) and AIBN (616.7 mg, 3.8 mmol). The reaction mixture was heated to reflux for 1 hour, allowed to cool to room temperature, and solids were filtered off. The filtrate was washed with saturated Na₂HCO₃ (aq, 2×100 ml), brine (100 mL), dried over anhydrous Na₂SO₄, and concentrated in vacuo to afford 9 as white crystal (14.76 g, 85.8%). Compound 9 was immediately used in the next synthetic step.

### 1-(4-hydroxybenzyl)indoline-2,3-dione (10)

Compound 9 (8.30 g, 36.2 mmol) was dissolved in DMF (10 ml) and added to a heterogenous mixture of isatin (5.86 g, 39.9 mmol), K₂CO₃ (11.02 g, 79.7 mmol) in DMF (25 mL). The reaction mixture was stirred at room temperature for 4 hours, quenched with DI (100 mL), and extracted with EA (2×100 mL). The combined organic extracts were washed with brine (100 mL), dried over anhydrous Na₂SO₄, and concentrated in vacuo to afford a red crystal. The residue was dissolved in MeOH (60 mL) and added to a RBF containing K₂CO₃ (10.02 g, 72.5 mmol) and stirred at room temperature for 2 hours. The reaction mixture was quenched with 1N HCl (50 mL), extracted with EA (2×100 mL), dried over anhydrous Na₂SO₄, concentrated in vacuo, and purified by flash chromatography (SiO₂, DCM/EA; 0-40%) to afford 10 as red crystal (2.30 g, 25.1%). ¹H NMR (DMSO-d₆, 600 MHz): δ 9.41 (1H, s); 7.59-7.54 (2H, m); 7.23-7.21 (2H, d, J=8.74 Hz); 7.11-7.08 (1H, m); 7.00-6.99 (1H, d, J=7.87 Hz); 6.71-6.70 (2H, d, J=8.74 Hz); 4.77 (2H, s). ¹³C NMR (DMSO-d₆, 600 MHz): δ183.7, 158.6, 157.3, 150.8, 138.4, 129.3, 125.9, 124.9, 123.7, 118.1, 115.8, 111.6, 42.9. Measured purity by elemental analysis: calcd for (C: 71.14, H: 4.38, N: 5.53); found (C: 70.65, H: 4.26, N: 5.54). HRMS: calcd for (C₁₅H₁₁NO₃): 253.0739, found 276.0641 (M+Na⁺).

### 1-(4-(2-(diethylamino)ethoxy)benzyl)indoline-2,3-dione (7)

1-(4-hydroxybenzyl)indoline-2,3-dione 10 (300.0 mg, 1.9 mmol) was dissolved in DMF (10 mL), added to a RBF containing Cs₂CO₃ (1.54 g, 4.7 mmol), and heated to 60° C. To the reaction mixture 2-chloro-N,N-diethylethan-1-amine hydrochloride (203.9 mg, 1.2 mmol) and NaI (cat.) were added and allowed to stir for 2 hours. The reaction mixture was cooled to 35° C. and quenched with 1N HCl (5 mL), then cooled to room temperature, washed with saturated NaHCO₃, and extracted with EA (3×20 mL). The organic extracts were washed with DI (30 mL), brine (30 mL), dried over anhydrous Na₂SO₄, concentrated in vacuo, and purified by flash chromatography (SiO₂, DCM/MeOH; 0-10%) to afford 7 as red wax (81.5 mg, 19.5%). ¹H NMR (CDCl₃, 600 MHz): δ 7.54-7.53 (1H, d, J=6.96 Hz); 7.46-7.43 (1H, m); 7.23-7.21 (2H, d, J=8.12 Hz); 7.05-7.02 (1H, m); 6.84-6.82 (2H, m); 6.78-6.76 (1H, d, J=8.12 Hz); 4.81 (2H, s); 4.00-3.97 (2H, m); 2.84-2.82 (2H, m); 2.62-2.58 (4H, m); 1.04-1.01 (6H, m). ¹³C NMR (CDCl₃, 600 MHz): δ 183.4, 158.6, 158.2, 150.7, 138.3, 128.8, 126.4, 125.3, 123.7, 117.6, 114.9, 111.0, 66.4, 51.5, 47.6, 43.5, 11.6. Measured purity by elemental analysis: calcd for (C: 71.57, H: 6.86, N: 7.95); found (C: 70.99, H: 6.69, N: 7.67). HRMS: calcd for (C₂₁H₂₄N₂O₃): 352.1787, found 353.1857 (M+H⁺).

### General Phenol Alkylation Procedure

The appropriate phenol (1.0 eq) was dissolved in DMF and added to a RBF containing K₂CO₃ (2.0-4.0 eq). After the reaction mixture was heated to 60° C. the corresponding alkyl halide (1.0-4.0 eq) was added and allowed to stir for 2 hours. After cooling to room temperature, the reaction mixture was quenched with DI and extracted with EA. The organic extracts were washed with DI, brine, dried over anhydrous Mg₂SO₄, concentrated in vacuo, and filtered through a pad of celite to afford the corresponding phenol ethers as described below.

### General Reduction Procedure

The appropriate benzaldehyde (1.0 eq) was dissolved in anhydrous MeOH and cooled to 0° C. NaBH₄ (1.5 eq) was added in 2 parts over 30 minutes. After stirring for an additional 30 minutes the reaction mixture was quenched with DI and extracted with EA. The organic extracts were washed with DI, brine, dried over anhydrous Na₂SO₄, concentrated in vacuo, and filtered through a pad of celite to afford the corresponding benzyl alcohols as described below.

### (3,5-diiodo-4-methoxyphenyl)methanol (11a)

Synthesized using the general phenol alkylation procedure with the following quantities: 4-hydroxy-3,5-diiodobenzaldehyde (2.00 g, 5.35 mmol); K₂CO₃ (2.96 g, 21.40 mmol); methyl iodide (1.33 mL, 21.40 mmol); afforded 3,5-diiodo-4-methoxybenzaldehyde as white solid (1.86 g, 89.4%). ¹H NMR (DMSO-d₆, 600 MHz): δ 9.83 (1H, s); 8.31 (2H, s); 3.81 (3H, s). ¹³C NMR (DMSO-d₆, 600 MHz): δ 190.3, 163.5, 141.0, 135.9, 92.9, 60.9.

Synthesized using the general reduction procedure with the following quantities: 3,5-diiodo-4-methoxybenzaldehyde (1.80 g, 4.64 mmol); NaBH₄ (263.3 mg, 6.96 mmol); afforded 11a as white solid (1.62 g, 89.7%). ¹H NMR (DMSO-d₆, 600 MHz): δ 7.74 (2H, s); 5.33-5.31 (1H, t, J=5.42 Hz); 4.40-4.39 (2H, d, J=4.70 Hz); 3.72 (3H, s). ¹³C NMR (DMSO-d₆, 600 MHz): δ157.4, 143.3, 137.8, 91.5, 61.1, 60.7.

### (3-iodo-4-methoxyphenyl)methanol (11b)

Synthesized using the general reduction procedure with the following quantities: 3-iodo-4-methoxybenzaldehyde (1.00 g, 3.82 mmol); NaBH₄ (216.6 mg, 5.72 mmol); afforded 11b as white solid (715.2 mg, 70.8%). ¹H NMR (DMSO-d₆, 600 MHz): δ7.70 (1H, d, J=2.06 Hz); 7.29-7.27 (1H, dd); 6.95-6.94 (1H, d, J=8.43 Hz); 5.16-5.14 (1H, t, J=7.13 Hz); 4.39-4.38 (2H, d, J=5.71 Hz); 3.79 (3H, s). ¹³C NMR (DMSO-d₆, 600 MHz): δ157.0, 137.6, 137.3, 128.4, 111.6, 86.1, 62.1, 56.8.

### (3,5-dibromo-4-methoxyphenyl)methanol (11c)

Synthesized using the general phenol alkylation procedure with the following quantities: 4-hydroxy-3,5-dibromobenzaldehyde (3.00 g, 10.72 mmol); K₂CO₃ (5.92 g, 42.87 mmol); methyl iodide (2.67 mL, 42.87 mmol); afforded 3,5-dibromo-4-methoxybenzaldehyde as off-yellow solid (2.83 g, 89.7%). ¹H NMR (DMSO-d₆, 600 MHz): δ 9.88 (1H, s); 8.16 (2H, s); 3.87 (3H, s). ¹³C NMR (DMSO-d₆, 600 MHz): δ 190.5, 158.5, 134.9, 134.2, 119.0, 61.2.

Synthesized using the general reduction procedure with the following quantities: 3,5-dibromo-4-methoxybenzaldehyde (2.80 g. 9.53 mmol); NaBH₄ (540.6 mg, 14.29 mmol); afforded 11c as off-yellow solid (2.77 g, 98.2%). ¹H NMR (DMSO-d₆, 600 MHz): δ 7.57 (2H, s); 5.41-5.39 (1H, m); 4.45-4.44 (2H, d, J=5.92 Hz); 3.77 (3H, s). ¹³C NMR (DMSO-d₆, 600 MHz): δ152.3, 142.6, 130.9, 117.6, 61.5, 60.8

### (3-bromo-4-methoxyphenyl)methanol (11d)

Synthesized using the general reduction procedure with the following quantities: 3-bromo-4-methoxybenzaldehyde (2.00 g, 9.30 mmol); NaBH₄ (527.8 mg, 13.95 mmol); afforded 11d as off-yellow oil (1.95 g, 96.5%). ¹H NMR (CDCl₃ DMSO-d₆, 600 MHz): δ 7.52 (1H, d, J=2.06 Hz); 7.27-7.26 (1H, dd); 7.02-7.01 (1H, d, J=8.45 Hz); 5.25-5.23 (1H, t, J=5.85 Hz); 4.45-4.44 (2H, d, J=6.67 Hz); 3.81 (3H, s). ¹³C NMR (CDCl₃ DMSO-d₆, 600 MHz): δ154.62, 136.7, 131.5, 127.5, 112.6, 110.7, 62.3, 56.5.

### (3,5-dichloro-4-methoxyphenyl)methanol (11e)

Synthesized using the general phenol alkylation procedure with the following quantities: 4-hydroxy-3,5-dichlorobenzaldehyde (1.00 g, 5.24 mmol); K₂CO₃ (2.90 g, 20.94 mmol); methyl iodide (1.35 mL, 20.94 mmol); afforded 3,5-dichloro-4-methoxybenzaldehyde as off-yellow oil (773.8 mg, 72.0%). ¹H NMR (DMSO-d₆, 600 MHz): δ 9.90 (1H, s); 8.01 (2H, s); 3.91 (3H, s). ¹³C NMR (DMSO-d₆, 600 MHz): δ 190.7, 156.6, 133.9, 130.5, 129.8, 61.4

Synthesized using the general reduction procedure with the following quantities: 3,5-dichloro-4-methoxybenzaldehyde (758.2 mg, 3.70 mmol); NaBH₄ (209.9 mg, 5.55 mmol); afforded 11e as off-yellow oil (639.4 mg, 83.5%). ¹H NMR (DMSO-d₆, 600 MHz): δ 7.38 (2H, s); 5.42-5.40 (1H, t, J=5.77 Hz); 4.45-4.44 (2H, d, J=6.17 Hz); 3.79 (3H, s). ¹³C NMR (DMSO-d₆, 600 MHz): δ150.4, 141.5, 128.4, 127.2, 61.7, 60.9.

### (3-chloro-4-methoxyphenyl)methanol (11f)

Synthesized using the general phenol alkylation procedure with the following quantities: 3-chloro-4-hydroxybenzaldehyde (880.0 mg, 5.62 mmol); K₂CO₃ (3.11 g, 22.48 mmol); methyl iodide (1.40 mL, 22.48 mmol); afforded 3-chloro-4-methoxybenzaldehyde as white solid (748.0 mg. 78.0%). ¹H NMR (CDCl₃, 600 MHz): δ 9.69 (1H, s); 7.71 (1H, d, J=1.95 Hz); 7.62-7.60 (1H, dd); 6.92-6.90 (1H, d, J=8.36 Hz); 3.84 (3H, s). ¹³C NMR (CDCl₃, 600 MHz): δ189.6, 159.6, 130.7, 130.6, 130.1, 123.4, 111.6, 56.4.

Synthesized using the general reduction procedure with the following quantities: 3-chloro-4-methoxybenzaldehyde (748.0 mg, 4.38 mmol); NaBH₄ (248.8 mg, 6.58 mmol); afforded 11f as clear oil (740.2 mg, 97.8%). ¹H NMR (CDCl₃, 600 MHz): δ 7.30-7.29 (1H, d, J=2.07 Hz); 7.14-7.12 (1H, dd); 6.85-6.83 (1H, d, J=8.27 Hz); 4.49 (2H, s); 3.84 (3H, s); 2.83 (1H, b). ¹³C NMR (CDCl₃, 600 MHz): δ 154.3, 134.1, 129.0, 126.5, 122.2, 111.9, 64.0, 56.1.

### (3,5-difluoro-4-methoxyphenyl)methanol (11g)

Synthesized using the general reduction procedure with the following quantities: 3,5-difluoro-4-methoxybenzaldehyde (600.0 mg, 3.49 mmol); NaBH₄ (197.8 mg, 5.23 mmol); afforded 11g as yellow oil (468.1 mg, 77.1%). ¹H NMR (DMSO-d₆, 600 MHz): δ 7.15-7.13 (1H, d, J=11.68 Hz); 7.08-7.04 (2H, m); 5.27-5.25 (1H, t, J=5.74 Hz); 4.46-4.45 (2H, d, J=5.81 Hz); 3.80 (3H, s). ¹³C NMR (DMSO-d₆, 600 MHz): δ156.2, 154.6, 139.5-139.4, 134.6-134.4, 110.4-110.2, 62.1, 61.9.

### (3-fluoro-4-methoxyphenyl)methanol (11h)

Synthesized using the general reduction procedure with the following quantities: 3-fluoro-4-methoxybenzaldehyde (1.00 g, 6.49 mmol); NaBH₄ (368.2 mg, 9.73 mmol); afforded 11h as yellow oil (984.4 mg, 97.5%). ¹H NMR (DMSO-d₆. 600 MHz): δ 7.15-7.13 (1H, d, J=11.74 Hz); 7.08-7.04 (2H, m); 5.27-5.25 (1H, t, J=5.76 Hz); 4.46-4.45 (2H, d, J=5.76 Hz); 3.80 (3H, s). ¹³C NMR (DMSO-d₆, 600 MHz): δ152.6, 151.0, 146.3, 146.2, 136.1, 136.0, 122.8, 114.5-114.4, 113.7, 62.5, 56.2.

### (3,4,5-trifluorophenyl)methanol (11i)

Synthesized using the general reduction procedure with the following quantities: 3,4,5-trifluorobenzaldehyde (2.00 g. 12.49 mmol); NaBH₄ (708.9 mg. 18.74 mmol); afforded 11i as yellow oil (1.93 g, 95.1%). ¹H NMR (DMSO-d₆, 600 MHz): δ 7.20 (2H, s); 5.50-5.48 (1H, t, J=5.77 Hz); 4.47-4.46 (2H, d, J=5.20 Hz). ¹³C NMR (DMSO-d₆, 600 MHz): δ151.4-151.3, 149.7-149.6, 140.6, 138.6-138.4, 136.9-136.7, 110.7-110.6, 61.7.

### (3,5-dibromo-4-(2-(diethylamino)ethoxy)phenyl)methanol (11l)

Synthesized using the general phenol alkylation procedure with the following quantities: 3,5-dibromo-4-hydroxybenzaldehyde (1.00 g, 3.57 mmol); K₂CO₃ (1.97 g, 14.29 mmol); 2-chloro-N,N-diethylethan-1-amine hydrochloride (614.8 mg, 3.57 mmol); afforded 3,5-dibromo-4-(2-(diethylamino)ethoxy)benzaldehyde as yellow oil (413.4 mg, 30.6%). ¹H NMR (CDCl₃, 600 MHz): δ 9.84 (1H, s); 8.02 (2H, s); 4.17-4.15 (2H, t, J=6.56 Hz); 3.03-3.01 (2H, t, J=6.40 Hz); 2.70-2.66 (4H, q, J=7.11 Hz); 1.09-1.07 (6H, t, J=7.22 Hz). ¹³C NMR (CDCl₃, 600 MHz): δ188.4, 158.4, 134.0, 133.9, 119.4, 71.7, 52.1, 47.5, 11.8.

Synthesized using the general reduction procedure with the following quantities: 3,5-dibromo-4-(2-(diethylamino)ethoxy)benzaldehyde (413.4 mg, 1.09 mmol); NaBH₄ (61.9 mg, 1.64 mmol); afforded 11l as yellow oil (360.3 mg, 86.7%). ¹H NMR (CDCl₃ DMSO-d₆, 600 MHz): δ 7.56 (2H, s); 5.40 (1H, b); 4.44 (2H, s); 3.97-3.95 (2H, t, J=6.39 Hz); 2.88-2.86 (2H, t, J=6.70 Hz); 2.59-2.56 (4H, q, J=7.31 Hz); 0.99-0.96 (6H, t, J=7.07 Hz). ¹³C NMR (CDCl₃ DMSO-d₆, 600 MHz): δ151.5, 142.4, 130.9, 117.7, 71.9, 61.5, 52.0, 47.5, 12.3.

### General Chlorination Procedure

The appropriate benzyl alcohol (1.0 eq) was dissolved in DCM and cooled to 0° C. Pyridine (1.3 eq) was added, followed by the addition of SOCl₂ (1.7 eq), and allowed to warm to room temperature over 16 hours. The reaction mixture was quenched with saturated NaHCO₃, extracted with DCM, washed with DI, brine, dried over anhydrous Na₂SO₄, and concentrated in vacuo to afford the corresponding crude benzyl chloride which was immediately used in the next reaction.

### 5-(chloromethyl)-1,3-diiodo-2-methoxybenzene (12a)

Synthesized using the general chlorination procedure with the following quantities: 11a (1.62 g, 4.15 mmol); pyridine (368.1 μL, 4.57 mmol); SOCl₂ (512.3 μL, 7.06 mmol); afforded 12a as yellow oil (1.37 g, 80.6%).

### 4-(chloromethyl)-2-iodo-1-methoxybenzene (12b)

Synthesized using the general chlorination procedure with the following quantities: 11b (750.0 mg, 2.84 mmol); pyridine (297.4 μL, 3.69 mmol); SOCl₂ (350.2 μL, 4.83 mmol); afforded 12b as yellow oil (crude, Th 802.4 mg).

### 1,3-dibromo-5-(chloromethyl)-2-methoxybenzene (12c)

Synthesized using the general chlorination procedure with the following quantities: 11c (2.50 g, 8.45 mmol); pyridine (748.5 μL, 9.29 mmol); SOCl₂ (1.04 mL, 14.36 mmol); afforded 12c as yellow oil (crude, Th 2.66 g).

### 2-bromo-4-(chloromethyl)-1-methoxybenzene (12d)

Synthesized using the general chlorination procedure with the following quantities: 11d (1.95 g, 8.98 mmol); pyridine (974.5 μL, 12.10 mmol); SOCl₂ (1.15 mL, 15.82 mmol); afforded 12d as yellow oil (crude, Th 2.19 g).

### 1,3-dichloro-5-(chloromethyl)-2-methoxybenzene (12e)

Synthesized using the general chlorination procedure with the following quantities: 11e (639.4 mg, 3.09 mmol); pyridine (323.4 μL, 4.01 mmol); SOCl₂ (380.8 μL, 5.25 mmol); afforded 12e as yellow oil (crude, Th 696.4 mg).

### 2-chloro-4-(chloromethyl)-1-methoxybenzene (12f)

Synthesized using the general chlorination procedure with the following quantities: 11f (740.2 mg, 4.29 mmol); pyridine (449.1 μL, 5.57 mmol); SOCl₂ (528.8 μL, 7.29 mmol); afforded 12f as yellow oil (crude, Th 819.29 mg).

### 5-(chloromethyl)-1,3-difluoro-2-methoxybenzene (12g)

Synthesized using the general chlorination procedure with the following quantities: 11g (461.1 mg, 2.69 mmol); pyridine (281.5 μL, 3.49 mmol); SOCl₂ (331.5 μL, 4.57 mmol); afforded 12g as yellow oil (crude, Th 517.7 mg).

### 4-(chloromethyl)-2-fluoro-1-methoxybenzene (12h)

Synthesized using the general chlorination procedure with the following quantities: 11h (984.4 mg, 6.30 mmol); pyridine (660.1 μL. 8.20 mmol); SOCl₂ (777.4 μL, 10.72 mmol); afforded 12h as yellow oil (crude, Th 1.10 g).

### 5-(chloromethyl)-1,2,3-trifluorobenzene (12i)

Synthesized using the general chlorination procedure with the following quantities: 11i (1.50 g, 9.25 mmol); pyridine (819.9 μL, 10.18 mmol); SOCl₂ (1.14 μL, 15.73 mmol); afforded 12i as yellow oil (crude, Th 1.67 g).

### 5-(bromomethyl)-1,2,3-trimethoxybenzene (12k)

(3,4,5-trimethoxyphenyl)methanol (2.03 mL, 12.61 mmol) and PPh₃ (4.96 g, 18.92 mmol) were dissolved in DCM (100 mL) and cooled to 0° C. CBr₄ (8.37 g, 25.22 mmol) was added to the reaction mixture and allowed to warm to room temperature over 16 hours. The reaction mixture was quenched with DI (100 mL) and extracted with DCM (2×75 mL). The combined organic extracts were washed with brine (75 mL), dried over anhydrous Mg₂SO₄, filtered through a pad of celite, concentrated in vacuo, and purified by flash chromatography (SiO₂, Hex/EA; 0-30%) to afford 12k as a white solid (2.40 g, 72.9%).

### 2-(2,6-dibromo-4-(chloromethyl)phenoxy)-N,N-diethylethan-1-amine hydrochloride (12l)

11l (415.6 mg, 1.09 mmol) was dissolved in anhydrous THF (10 mL) and cooled to 0° C. SOCl₂ (118.7 μL, 1.64 mmol) was added dropwise and allowed to stir for 2 hours. The reaction mixture was diluted with diethyl ether (DE, 20 mL) and cooled to −10° C. with no stirring, resulting in an oil precipitate. The residual solution was decanted off affording 12i as a clear oil (crude, Th 474.5 mg).

### General N-alkylation Procedure

Isatin (1.0 eq) was dissolved in DMF and added to a RBF containing K₂CO₃ (2.0 eq), KI (1.0 eq), and stirred at room temperature. To the reaction mixture the appropriate benzyl halide (1.15-2.7 eq) was added and stirred for 16 hours. The reaction mixture was quenched with 1N HCl (2.0 eq) and extracted with EA. The combined organic extracts were washed with DI, brine, dried over anhydrous Na₂SO₄, and concentrated in vacuo. The residue was recrystallized from EA/Hex to afford the corresponding N-alkylated derivative.

### 1-(3,5-diiodo-4-methoxybenzyl)indoline-2,3-dione (8a)

Synthesized using the general N-alkylation procedure with the following quantities: 12a (682.8 mg, 1.67 mmol); isatin (205.0 mg, 1.39 mmol); K₂CO₃ (385.1 mg, 2.79 mmol); KI (231.3 mg, 1.39 mmol); afforded 8a as red solid (356.8 mg, 49.3%). ¹H NMR (DMSO-d₆, 600 MHz): δ 7.93 (2H, s); 7.60-7.56 (2H, in); 7.13-7.10 (1H, m); 6.98-6.96 (1H, d, J=8.06 Hz); 4.81 (2H, s); 3.71 (3H, s). ¹³C NMR (DMSO-d₆, 600 MHz): δ183.2, 159.0, 158.1, 150.3, 138.7, 138.1, 136.2, 124.8, 123.7, 118.5, 111.2, 92.0, 60.6, 41.3. Measured purity at 254 nm: 97.6%; 280 nm: 98.2%. HRMS: calcd for (C₁₆H₁₁I₂NO₃): 518.8828, found 519.8903 (M+H⁺).

### 1-(3-iodo-4-methoxybenzyl)indoline-2,3-dione (8b)

Synthesized using the general N-alkylation procedure with the following quantities: 12b (802.4 mg, 2.84 mmol); isatin (300.0 mg, 2.04 mmol); K₂CO₃ (563.59 mg, 4.08 mmol); KI (338.5 mg, 2.04 mmol); afforded 8b as red solid (437.3 mg, 54.5%). ¹H NMR (DMSO-d₆, 600 MHz): δ 7.86 (1H, d, J=2.28 Hz); 7.59-7.55 (2H, m); 7.44-7.43 (1H, dd); 7.12-7.09 (1H, t, J=8.06 Hz); 6.99-6.94 (2H, in); 4.81 (2H, s); 3.79 (3H, s). ¹³C NMR (DMSO-d₆, 600 MHz): δ183.5, 158.8, 157.6, 150.6, 138.3, 138.2, 130.0, 129.5, 124.8, 123.7, 118.3, 111.9, 111.4, 86.8, 56.8, 42.0. Measured purity at 254 nm: 99.7%; 280 nm: 99.5%. HRMS: calcd for (C₁₆H₁₂INO₃): 392.9862, found 393.9939 (M+H⁺).

### 1-(3,5-dibromo-4-methoxybenzyl)indoline-2,3-dione (8c)

Synthesized using the general N-alkylation procedure with the following quantities: 12c (2.66 g, 8.45 mmol); isatin (500.0 mg. 3.4 mmol); K₂CO₃ (939.3 mg, 6.80 mmol); KI (564.1 mg, 3.4 mmol); afforded 8c as red solid (708.7 mg, 49.2%). ¹H NMR (DMSO-d₆, 600 MHz): δ 7.79 (2H, s); 7.60-7.56 (2H, m); 7.13-7.11 (1H, m); 6.97-6.96 (1H, d, J=7.94 Hz); 4.86 (2H, s); 3.76 (3H, s). ¹³C NMR (DMSO-d₆, 600 MHz): δ183.2, 159.0, 153.1, 150.3, 138.1, 135.5, 132.1, 124.8, 123.7, 118.6, 118.0, 111.2, 60.8, 41.7. Measured purity at 254 nm: 98.1%; 280 nm: 96.7%. HRMS: calcd for (C₁₆H₁₁Br₂NO₃): 422.9106, found 423.9189 (M+H⁺).

### 1-(3-bromo-4-methoxybenzyl)indoline-2,3-dione (8d)

Synthesized using the general N-alkylation procedure with the following quantities: 12d (2.19 g, 9.31 mmol); isatin (500.0 mg, 3.40 mmol); K₂CO₃ (939.3 mg, 6.80 mmol); KI (564.1 mg, 3.40 mmol); afforded 8d as red solid (478.2 mg, 40.5%). ¹H NMR (CDCl₃, 600 MHz): δ 7.63-7.62 (1H, m); 7.54-7.50 (2H, m); 7.27-7.25 (2H, m); 7.13-7.10 (1H, in); 6.87-8.86 (1H, d, J=8.20 Hz); 6.79-6.78 (1H, d, J=7.52 Hz); 4.85 (2H, s); 3.88 (3H, s). ¹³C NMR (CDCl₃, 600 MHz): δ 183.0, 158.2, 155.8, 150.4, 138.3, 132.4, 127.9, 127.8, 125.7, 125.5, 124.0, 121.5, 117.7, 112.2, 112.1, 110.8, 56.3, 42.9. Measured purity at 254 nm: 99.3%; 280 nm: 98.0%. HRMS: calcd for (C₁₆H₁₂BrNO₃): 345.0001, found 346.0075 (M+H⁺).

### 1-(3,5-dichloro-4-methoxybenzyl)indoline-2,3-dione (8e)

Synthesized using the general N-alkylation procedure with the following quantities: 12e (696.4 mg, 3.09 mmol); isatin (300.0 mg, 2.04 mmol); K₂CO₃ (563.6 mg, 4.08 mmol); KI (338.5 mg, 2.04 mmol); afforded 8e as red solid (312.9 mg, 45.6%). ¹H NMR (DMSO-d₆, 600 MHz): δ 7.63 (2H, s); 7.59-7.56 (2H, m); 7.13-7.11 (1H, td); 6.97-6.95 (1H, d, J=7.91 Hz); 4.87 (1H, s); 3.79 (3H, s). ¹³C NMR (DMSO-d₆, 600 MHz): δ183.2, 159.0, 151.2, 150.2, 138.1, 134.5, 128.8, 128.5, 124.8, 123.7, 118.5, 111.2, 61.0, 42.0. Measured purity at 254 nm: 98.7%; 280 nm: 97.4%. HRMS: calcd for (C₁₆H₁₁Cl₂NO₃): 335.0116, found 336.0196 (M+H⁺).

### 1-(3-chloro-4-methoxybenzyl)indoline-2,3-dione (8f)

Synthesized using the general N-alkylation procedure with the following quantities: 12f (819.29 mg, 4.29 mmol); isatin (400.0 mg, 2.72 mmol); K₂CO₃ (751.5 mg, 5.44 mmol); KI (451.3 mg, 2.72 mmol); afforded 8f as red solid (326.7 mg, 39.8%). ¹H NMR (CDCl₃, 600 MHz): δ 7.62-7.61 (1H, m); 7.52-7.49 (1H, m); 7.36-7.35 (1H, d, J=2.32 Hz); 7.21-7.20 (1H, dd); 7.12-7.09 (1H, td); 6.89-6.88 (1H, d, J=8.35 Hz); 6.78-6.77 (1H, d, J=7.99 Hz); 4.84 (2H, s); 3.88 (3H, s). ¹³C NMR (CDCl₃, 600 MHz): δ 183.0, 158.2, 154.9, 150.4, 138.3, 129.4, 127.5, 127.0, 125.5, 124.0, 123.0, 117.7, 112.4, 110.8, 56.2, 43.0. Measured purity at 254 nm: 99.8%; 280 nm: 99.7%. HRMS: calcd for (C₁₆H₁₂ClNO₃): 301.0506, found 302.0590 (M+H⁺).

### 1-(3,5-difluoro-4-methoxybenzyl)indoline-2,3-dione (8g)

Synthesized using the general N-alkylation procedure with the following quantities: 12g (517.7 mg, 2.69 mmol); isatin (300.0 mg, 2.04 mmol); K₂CO₃ (563.6 mg, 4.08 mmol); KI (338.5 mg, 2.04 mmol); afforded 8g as red solid (211.2 mg, 34.2%). ¹H NMR (DMSO-d₆, 600 MHz): δ 7.59-7.56 (2H, m); 7.31-7.27 (2H, m); 7.13-7.10 (1H, td); 6.93-6.92 (1H, m); 4.85 (2H, s); 3.88 (3H, s). ¹³C NMR (DMSO-d₆, 600 MHz): δ183.2, 158.9, 156.3, 154.7-154.6, 150.3, 138.1, 135.4-135.2, 132.2-132.1, 124.8, 123.7, 118.5, 112.0-111.9, 111.2, 62.2, 42.2. Measured purity at 254 nm: 97.6%; 280 nm: 97.9%. HRMS: calcd for (C₁₆H₁₁F₂NO₃): 303.0707, found 304.0782 (M+H⁺).

### 1-(3-fluoro-4-methoxybenzyl)indoline-2,3-dione (8h)

Synthesized using the general N-alkylation procedure with the following quantities: 12h (1.10 g, 6.30 mmol); isatin (500.0 mg, 3.40 mmol); K₂CO₃ (939.3 mg, 6.80 mmol); KI (564.1 mg, 3.40 mmol); afforded 8h as red solid (608.6 mg, 62.8%). ¹H NMR (DMSO-d₆, 600 MHz): δ 7.48-7.55 (2H, m); 7.34-7.31 (1H, dd); 7.23-7.21 (1H, dd); 7.13-7.09 (2H, m); 6.96-6.95 (1H, d, J=7.49 Hz); 4.83 (2H, s); 3.80 (3H, s). ¹³C NMR (DMSO-d₆, 600 MHz): δ182.4, 158.8, 152.6, 151.0, 150.5, 146.9, 138.2, 128.7, 124.8-123.7, 118.3, 115.7-115.5, 114.3, 111.4, 56.4, 42.4. Measured purity at 254 nm: 99.9%; 280 nm: 99.6%. HRMS: calcd for (C₁₆H₁₂FNO₃): 285.0801, found 286.0883 (M+H⁺).

### 1-(3,4,5-trifluorobenzyl)indoline-2,3-dione (8i)

Synthesized using the general N-alkylation procedure with the following quantities: 12i (1.67 g, 9.25 mmol); isatin (500.0 mg, 3.40 mmol); K₂CO₃ (939.3 mg, 6.80 mmol); KI (564.1 mg, 3.40 mmol); afforded 8i as red solid (318.1 mg, 32.1%). ¹H NMR (CDCl₃, 600 MHz): δ 7.66-7.65 (1H, m); 7.56-7.53 (1H, m); 7.17-7.14 (1H, td); 6.99-6.96 (2H, t, J=7.10 Hz); 6.74-6.73 (1H, d, J=7.81 Hz); 4.86 (2H, s). ¹³C NMR (CDCl₃, 600 MHz): δ 182.4, 158.1, 152.4, 152.3, 150.7, 150.6, 149.8, 140.4, 138.7, 138.5, 130.9, 130.8, 125.8, 124.4, 117.7, 111.6, 111.5, 110.4, 42.9. Measured purity at 254 nm: 99.9%; 280 nm: 98.9%. HRMS: calcd for (C₁₅H₈F₃NO₂): 291.0507, found 292.0593 (M+H⁺).

### 1-(3-methoxybenzyl)indoline-2,3-dione (8j)

Synthesized using the general N-alkylation procedure with the following quantities: 12j (1.01 mL, 7.48 mmol); isatin (500.0 mg, 3.40 mmol); K₂CO₃ (939.3 mg, 6.80 mmol); KI (564.1 mg, 3.40 mmol); afforded 8j as red solid (321.7 mg, 35.4%). ¹H NMR (CDCl₃, 600 MHz): δ 7.62-7.60 (1H, m); 7.49-7.46 (1H, td); 7.27-7.25 (2H, m); 7.10-7.07 (1H, td); 6.92-6.90 (1H, dd); 6.86-6.82 (2H, m); 6.79-6.77 (1H, d, J=7.93 Hz); 4.90 (2H, s); 3.78 (3H, s). ¹³C NMR (CDCl₃, 600 MHz): δ183.2, 160.1, 158.2, 150.7, 138.3, 136.0, 130.1, 125.4, 123.8, 119.6, 117.6, 113.3, 113.2, 111.0, 55.5, 44.0. Measured purity at 254 nm: 100.0%; 280 nm: 99.1%. HRMS: calcd for (C₁₆H₁₃NO₃): 267.0895, found 290.0789 (M+Na⁺).

### 1-(3,4,5-trimethoxybenzyl)indoline-2,3-dione (8k)

Synthesized using the general N-alkylation procedure with the following quantities: 12k (1.02 g, 3.91 mmol); isatin (500.0 mg, 3.40 mmol); K₂CO₃ (939.3 mg, 6.80 mmol); KI (564.1 mg. 3.40 mmol); afforded 8k as red solid (507.4 mg, 45.7%). ¹H NMR (CDCl₃, 600 MHz): δ 7.63-762 (1H, dd); 7.53-7.50 (1H, td); 7.12-7.10 (1H, td), 6.83-6.81 (1H, d, J=7.81 Hz); 6.53 (2H, s); 4.85 (2H, s); 3.82 (6H, s); 3.82 (3H, s). ¹³C NMR (CDCl₃, 600 MHz): δ183.2, 158.3, 153.7, 150.7, 138.3, 137.8, 130.1, 125.5, 124.0, 117.6, 111.0, 104.5, 60.9, 56.2, 44.4. Measured purity at 254 nm: 100.0%; 280 nm: 99.3%. HRMS: calcd for (C₁₈H₁₇NOs): 327.1107, found 328.1201 (M+H⁺).

### 1-(3,5-dibromo-4-(2-(diethylamino)ethoxy)benzyl)indoline-2,3-dione hydrochloride (8l)

Synthesized using the general N-alkylation procedure with the following quantities: 12l (475.5 mg, 1.09 mmol); isatin (160.5 mg, 1.09 mmol); K₂CO₃ (452.1 mg, 3.27 mmol); KI (181.0 mg, 1.09 mmol). The crude material was purified by flash chromatography (SiO₂, DCM/McOH; 0-5%) to afford 8l as red wax (45.2 mg, 8.1%). ¹H NMR (CDCl₃, 600 MHz): δ 7.61-7.65 (1H, dd); 7.57-7.54 (1H, td); 7.48 (2H, s); 7.17-7.14 (1H, td); 6.78-6.77 (1H, d, J=7.49 Hz); 4.83 (2H, s); 4.09-4.07 (2H, t, J=6.40 Hz); 3.01 (2H, s); 2.70 (4H, s); 1.10-1.07 (6H, t, J=6.99 Hz). ¹³C NMR (CDCl₃, 600 MHz): δ182.6, 158.1, 153.4, 150.0, 138.5, 133.0, 131.5, 125.7, 124.3, 119.0, 117.7, 110.6, 52.0, 47.5, 42.5, 29.7, 11.7. Measured purity by elemental analysis: calcd for (C: 49.44, H: 4.35, N: 5.49, Br: 31.32); found (C: 49.43, H: 4.32, N: 5.49, Br: 31.09). HRMS: calcd for (C₂₁H₂₃Br₂N₂O₃): 507.9997, found 509.0086 (M+H⁺).

Certain embodiments of the compositions and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

1. A composition comprising Formula I: 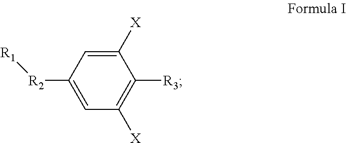
wherein:
each X is, independently, H, F, Cl, Br, I, OCH₃, CF₃, or OCF₃;
R₁ is selected from 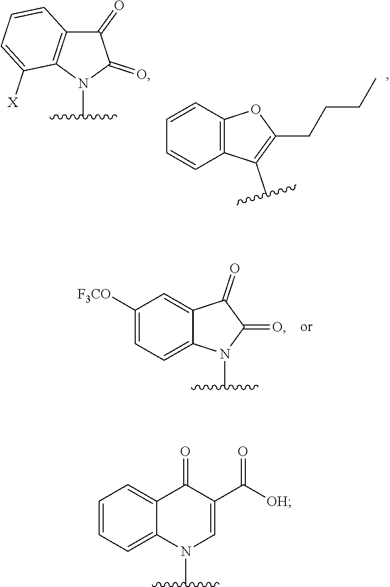
R₂ is selected from either 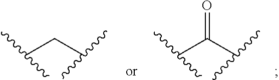
and
R₃ is H, F, Br, OCF₃, OCH₃, 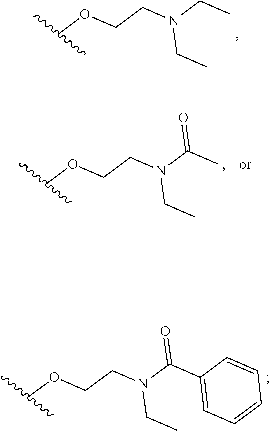
or a salt, stereoisomer, racemate, hydrate, solvate, polymorph, or prodrug thereof.

2. The composition of claim 1, comprising compound 5a:

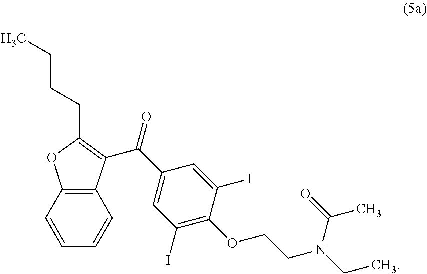 [SMILES: CCCCc1oc2ccccc2c1C(=O)c1cc(I)c(OCCN(CC)C(C)=O)c(I)c1]

3. The composition of claim 1, comprising compound 5b:

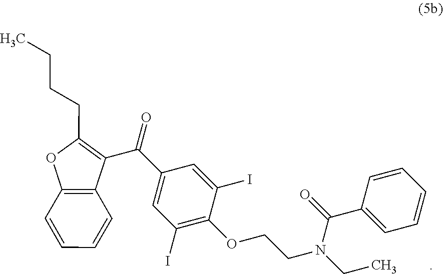 [SMILES: CCCCc1oc2ccccc2c1C(=O)c1cc(I)c(OCCN(CC)C(=O)c2ccccc2)c(I)c1]

4. The composition of claim 1, comprising compound 7:

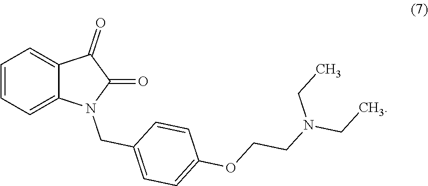 [SMILES: CCN(CC)CCOc1ccc(CN2C(=O)C(=O)c3ccccc32)cc1]

5. The composition of claim 1, comprising compound 8a:

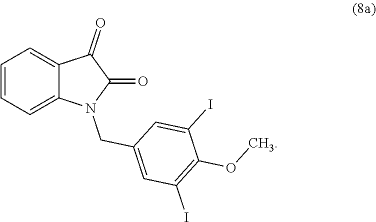 [SMILES: COc1c(I)cc(CN2C(=O)C(=O)c3ccccc32)cc1I]

6. The composition of claim 1, comprising compound 8c:

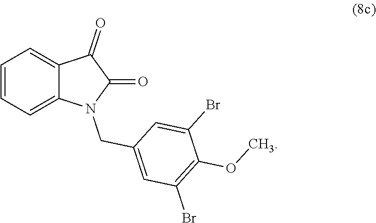 [SMILES: COc1c(Br)cc(CN2C(=O)C(=O)c3ccccc32)cc1Br]

7. The composition of claim 1, comprising compound 8l:

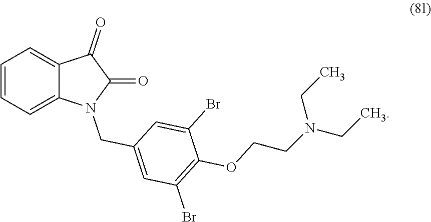 [SMILES: CCN(CC)CCOc1c(Br)cc(CN2C(=O)C(=O)c3ccccc32)cc1Br]

8. The composition of claim 1, comprising compound 8j:

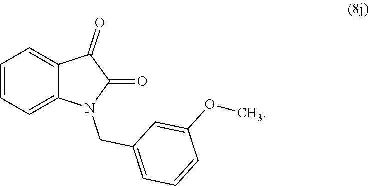 [SMILES: COc1cccc(CN2C(=O)C(=O)c3ccccc32)c1]

9. The composition of claim 1, comprising compound 8e:

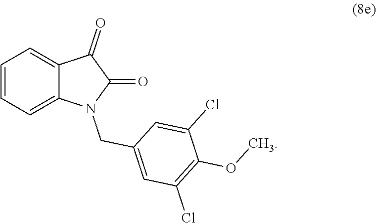 [SMILES: COc1c(Cl)cc(CN2C(=O)C(=O)c3ccccc32)cc1Cl]

10. The composition of claim 1, comprising compound 8g:

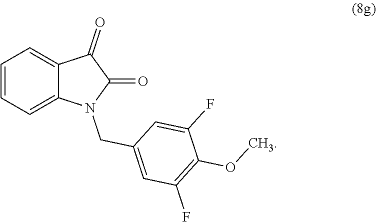 [SMILES: COc1c(F)cc(CN2C(=O)C(=O)c3ccccc32)cc1F]

11. The composition of claim 1, comprising compound GK-1-44:

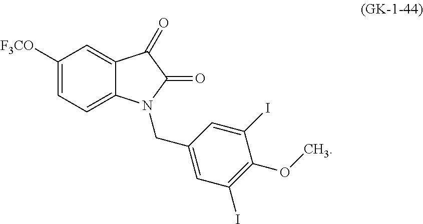 [SMILES: COc1c(I)cc(CN2C(=O)C(=O)c3cc(OC(F)(F)F)ccc32)cc1I]

12. The composition of claim 1, comprising compound GK-1-48:

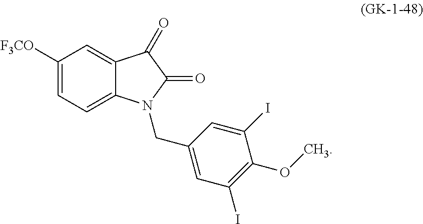 [SMILES: COc1c(I)cc(CN2C(=O)C(=O)c3cc(OC(F)(F)F)ccc32)cc1I]

13. The composition of claim 1, comprising compound GK-1-47:

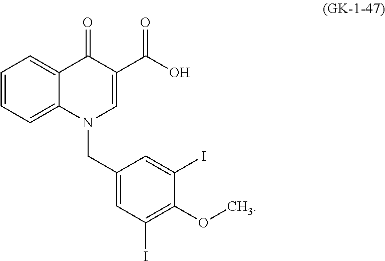 [SMILES: COc1c(I)cc(Cn2cc(C(=O)O)c(=O)c3ccccc32)cc1I]

14. The composition of claim 1, comprising compound GK-1-50:

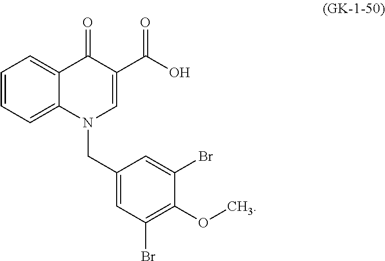 [SMILES: COc1c(Br)cc(Cn2cc(C(=O)O)c(=O)c3ccccc32)cc1Br]

15-24. (canceled)

25. A method of modulating ACh activity at M₁ muscarinic receptors, the method comprising contacting cells expressing M₁ muscarinic receptors with an effective amount of a compound of Formula I to modulate ACh activity at the M₁ muscarinic receptors expressed by the cells: 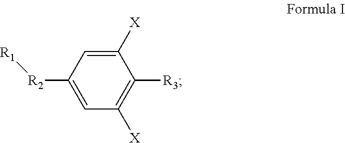
wherein:
each X is, independently, H, F, Cl, Br, I, OCH₃, CF₃, or OCF₃;
R₁ is selected from 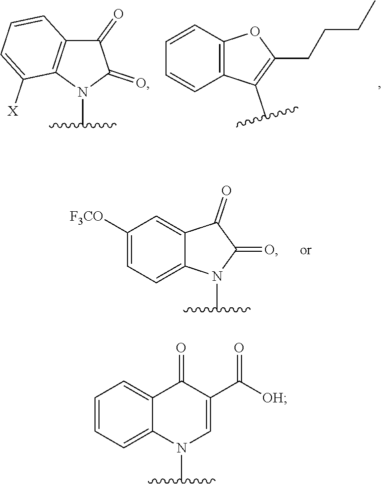
R₂ is selected from either 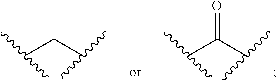
and
R₃ is H, F, Br, OCF₃,

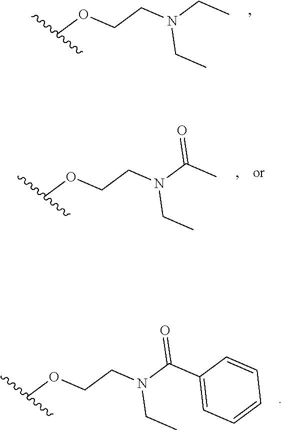

26-31. (canceled)

32. The method of claim 25, wherein the compound is selected from the group consisting of compound 5a, compound 5b, compound 7, compound 8a, compound 8c, compound 8e, compound 8g, compound 8l, compound 8j, compound GK-1-44, compound GK-1-47, compound GK-1-48, and compound GK-1-50:

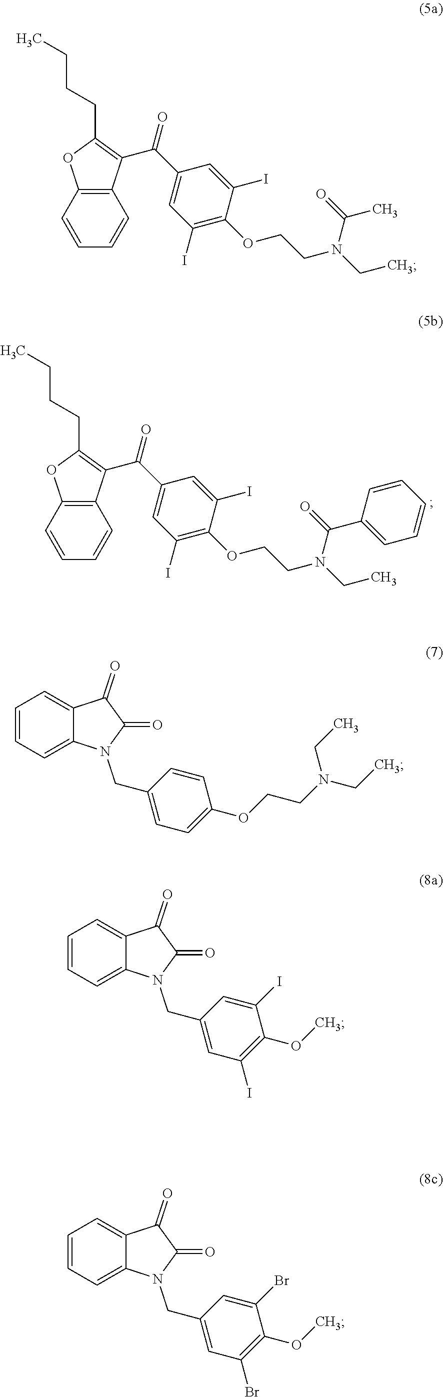 [SMILES: CCCCc1oc2ccccc2c1C(=O)c1cc(I)c(OCCN(CC)C(C)=O)c(I)c1 ; CCCCc1oc2ccccc2c1C(=O)c1cc(I)c(OCCN(CC)C(=O)c2ccccc2)c(I)c1 ; CCN(CC)CCOc1ccc(CN2C(=O)C(=O)c3ccccc32)cc1 ; COc1c(I)cc(CN2C(=O)C(=O)c3ccccc32)cc1I ; COc1c(Br)cc(CN2C(=O)C(=O)c3ccccc32)cc1Br]

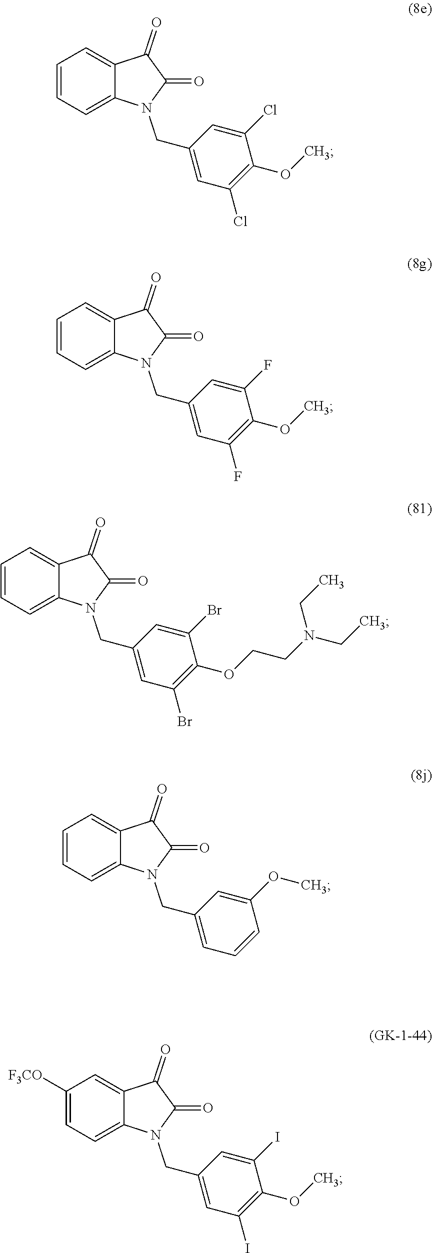 [SMILES: COc1c(Cl)cc(CN2C(=O)C(=O)c3ccccc32)cc1Cl ; COc1c(F)cc(CN2C(=O)C(=O)c3ccccc32)cc1F ; CCN(CC)CCOc1c(Br)cc(CN2C(=O)C(=O)c3ccccc32)cc1Br ; COc1cccc(CN2C(=O)C(=O)c3ccccc32)c1 ; COc1c(I)cc(CN2C(=O)C(=O)c3cc(OC(F)(F)F)ccc32)cc1I]

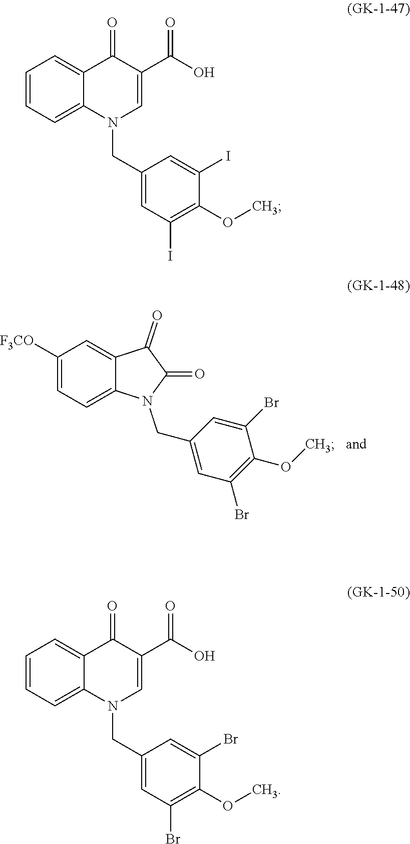

33-44. (canceled)

45. A method of decreasing repetitive behaviors in a subject, the method comprising administering an effective amount of a compound of Formula I to a subject and decreasing repetitive behaviors in the subject: 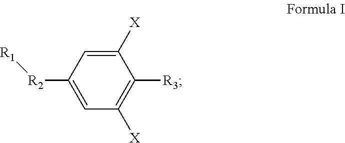
wherein:
each X is, independently, H, F, Cl, Br, I, OCH₃, CF₃, or OCF₃;
R₁ is selected from 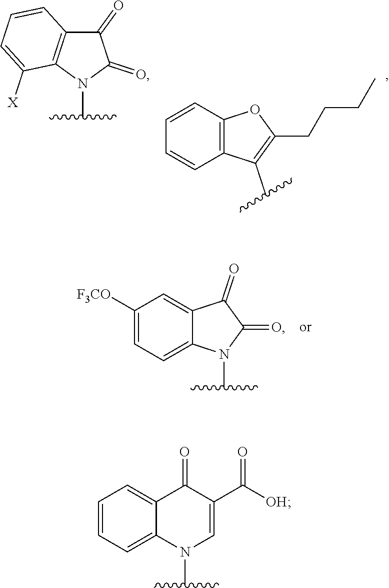
R₂ is selected from either 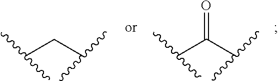
and
R₃ is H, F, Br, OCF₃,

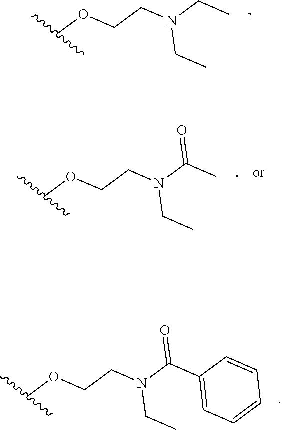

46. The method of claim 45, wherein the compound is selected from the group consisting of compound 8a, compound 8c, compound 8e, compound 8g, compound 8l, compound 8j, and compound GK-1-48:

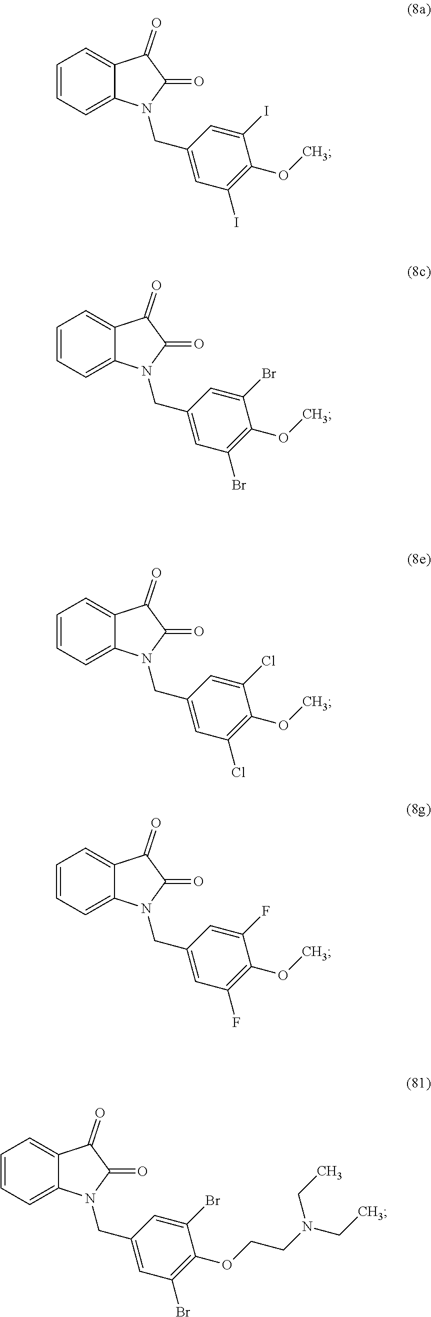 [SMILES: COc1c(I)cc(CN2C(=O)C(=O)c3ccccc32)cc1I ; COc1c(Br)cc(CN2C(=O)C(=O)c3ccccc32)cc1Br ; COc1c(Cl)cc(CN2C(=O)C(=O)c3ccccc32)cc1Cl ; COc1c(F)cc(CN2C(=O)C(=O)c3ccccc32)cc1F ; CCN(CC)CCOc1c(Br)cc(CN2C(=O)C(=O)c3ccccc32)cc1Br]

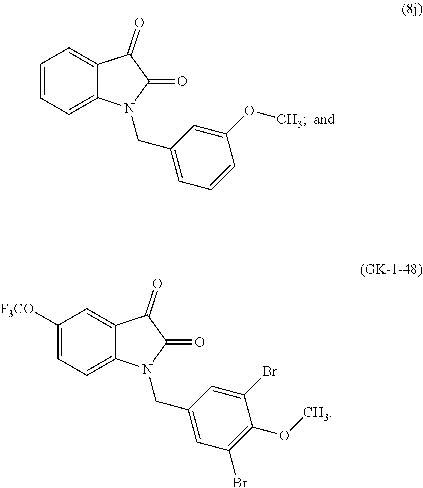

47-53. (canceled)

54. The method of claim 45, wherein the subject has a neurological disorder selected from the group consisting of Alzheimer's disease, Parkinson's disease, schizophrenia, autism spectrum disorders, substance abuse, and post-traumatic stress disorder.

55-69. (canceled)

70. The method of claim 54, wherein the neurological disorder is post-traumatic stress disorder, and the compound is compound 7:

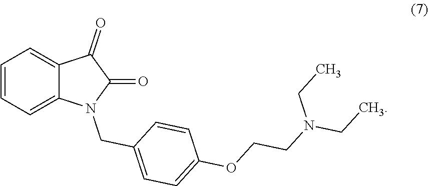 [SMILES: CCN(CC)CCOc1ccc(CN2C(=O)C(=O)c3ccccc32)cc1]

71. The method of claim 54, wherein the neurological disorder is Alzheimer's disease, and the compound is compound 8a:

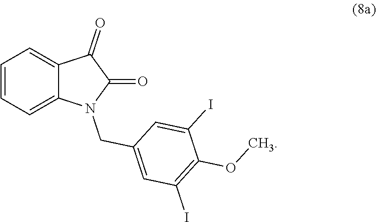 [SMILES: COc1c(I)cc(CN2C(=O)C(=O)c3ccccc32)cc1I]

72-80. (canceled)
